(19) United States
(12) Patent Application Publication
(10) Pub. No.: US 2025/0361299 A1
(43) Pub. Date: Nov. 27, 2025

(54) PROTEASE CLEAVABLE RECOMBINANT BISPECIFIC ANTIBODIES AND COMPOSITIONS AND USES THEREOF

(71) Applicant: Zhejiang Shimai Pharmaceutical Co., Ltd., Hangzhou, Zhejiang (CN)

(72) Inventors: Zuoxiang Xiao, Hangzhou, Zhejiang (CN); Jiaping Peng, Hangzhou, Zhejiang (CN); Dongwen Zhou, Hangzhou, Zhejiang (CN); Wei Zhou, Hangzhou, Zhejiang (CN)

(73) Assignee: Zhejiang Shimai Pharmaceutical Co., Ltd., Hangzhou, Zhejiang (CN)

(21) Appl. No.: 18/864,730

(22) PCT Filed: Nov. 28, 2022
(86) PCT No.: PCT/CN2022/134707

§ 371 Date: Nov. 11, 2024

(51) Int. Cl. C07K 16/28; A61K 39/00; A61P 35/00

(52) U.S. Cl. CPC C07K 16/2809; A61P 35/00; C07K 16/2863; A61K 2039/505; C07K 2317/31; C07K 2317/55; C07K 2317/622; C07K 2317/73; C07K 2317/76; C07K 2317/92; C07K 2317/94; C07K 2319/50

(57) ABSTRACT

Provided herein are protease cleavable recombinant bispecific antibodies and compositions and uses thereof, specifically bispecific antibodies, nucleic acids including nucleotide sequences encoding the bispecific antibodies, vectors including the nucleic acids, host cells including the nucleic acids or the vectors, pharmaceutical compositions and conjugates including the bispecific antibodies, and therapeutic methods for using the bispecific antibodies.

### CROSS-REFERENCE TO RELATED APPLICATIONS

This application is a national stage filing under 35 U.S.C. 371 of International Patent Application Serial No. PCT/CN2022/134707, filed Nov. 28, 2022. The entire contents of which are incorporated herein by reference.

### REFERENCE TO AN ELECTRONIC SEQUENCE LISTING

The contents of the electronic sequence listing (L047370059US00-SEQ-WWZ.xml; Size: 139,744 bytes; and Date of Creation: Nov. 8, 2024) are herein incorporated by reference in its entirety.

### FIELD OF THE INVENTION

The present invention is directed to recombinant bispecific antibodies, and uses of such antibodies, in particular their use in the treatment of cancers.

### BACKGROUND OF THE INVENTION

Antibodies are gammaglobulin proteins, predominantly referred to as immunoglobulins (Ig). A monomeric antibody is composed of two heavy chains and two light chains. The amino termini of the polypeptide chains show considerable variation in amino acid composition and are referred to as the variable (V) domains/regions to distinguish them from the relatively constant (C) domains/regions. Each light chain has a variable domain and a constant domain. Each heavy chain has four domains: a variable domain, and constant domains 1, 2, and 3. The antigen binding site is located in the Fab (Fragment antigen binding) region which includes a light chain variable domain (VL), a heavy chain variable domain (VH), a light chain constant domain (CL) and a heavy chain constant domain 1 (CH1). The combination of a light chain variable domain (VL) and a heavy chain variable domain (VH) is called Fv (Fragment variable) region. The Fc (Fragment, Crystallizable) region of an antibody includes heavy chain constant domains 2 and 3 (CH2 and CH3).

Protease-activatable bispecific proteins developed by Amgen (U.S. patent application publication No. 2017/0247476) use two scFvs as antigen-binding fragments, CD3e domain to mask CD3 binding, CH1-CL for heterodimerization, and wild-type IgG Fc or Fc with heterodimerizing alterations such as knob-in-hole mutations to avoid homodimerization. The use of scFvs, however, could cause aggregation and instability of the proteins. Although CH1-CL had been used as a heterodimerization scaffold to generate bispecific antibodies or multivalent fusion proteins, cooperation between the VH-VL and CH1-CL interfaces is required for mutual stabilization. In the absence of VH-VL, it has been reported that CH1-CL is insufficient to yield heterodimeric products. Furthermore, Fc with currently known heterodimerizing alterations has failed to form heterodimers completely.

The protease-activated T cell bispecific molecules developed by Roche (international application publication No. WO2017/162587) are based on the above-mentioned CrossMab platform and uses an idiotype-specific scFv as a masking moiety to block binding of the CD3 antibody. This design inherits the shortcomings associated with Cross Mab platform and scFv fragments described above.

Therefore, there is a need for bispecific antibody formats that can overcome the shortcomings in the art.

### SUMMARY OF THE INVENTION

The present invention provides engineered bispecific antibodies designed for simultaneous binding to a surface antigen on target cells and to an activating component on immune cells such as T cells. In one embodiment, the bispecific antibodies of the present invention comprise Fv or scFv and Fab as antigen-binding fragments and a modified Fc region which result in excellent properties of final products, such as 100% heterodimerization, high yield, high stability, low aggregation propensities and extended half-lives. In another embodiment, the present bispecific antibodies may also comprise protease cleavage sites and/or motifs that could be digested by certain tumor-associated proteases, resulting in the exposure of the binding site for immune cell receptors leading to the subsequent immune responses in the tumor microenvironment.

In an aspect, the present invention provides a bispecific antibody comprising a first binding region that binds to CD3 comprising a first heavy chain variable region (VH1) and a first light chain variable region (VL1), and a second binding region that binds to a tumor associated antigen (TAA) comprising a second heavy chain variable region (VH2) and a second light chain variable region (VL2); wherein the first binding region binds to an epitope of CD3 comprising, within or overlapping with an amino acid sequence of QDGNEEMGGITQTPYKVSISGT (SEQ ID NO: 1); wherein the VH1 and/or the VL1 is linked to a mask peptide via a first linker; wherein the mask peptide comprises the amino acid sequence of SEQ ID NO: 1 or a fragment thereof with at least 5 amino acids in length; and wherein the first linker comprises a protease cleavage site that is specifically cleaved by a protease expressed or overexpressed in tumor microenvironment.

In some embodiments of the bispecific antibody disclosed herein, the mask peptide comprises a fragment of at least 5 amino acids, preferably at least 7 amino acids, such as 7-15 amino acids, at N terminal of SEQ ID NO: 1.

In some embodiments, the mask peptide comprises an amino acid sequence as set forth in any of SEQ ID NOs: 1-4, or any combination thereof.

In some embodiments, the mask peptide comprises:

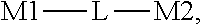

wherein M1 and M2 are each independently comprises an amino acid sequence as set forth in any of SEQ ID NOs: 1-4, and L is an optional linker, preferably the linker is (GGGS)n or (GGGGS)n, wherein n is an integer selected from 1-4.

In some embodiments, the mask peptide comprises an amino acid sequence selected from SEQ ID NOs: 5-8.

In some embodiments, the mask peptide comprises:

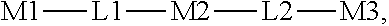

wherein M1, M2 and M3 are each independently comprises an amino acid sequence as set forth in any of SEQ ID NOs: 1-4, and each of L1 and L2 is an optional linker, preferably L1 and L2 are each independently (GGGS)n or (GGGGS)n, wherein n is an integer selected from 1-4.

In some embodiments, the VH1 comprises HCDRs 1-3 having the amino acid sequences as set forth in SEQ ID NOs: 9-11 respectively, and the VL1 comprises LCDRs 1-3 having the amino acid sequences as set forth in SEQ ID NOs: 12-14 respectively.

In some embodiments, the VH1 comprises an amino acid sequence as set forth in SEQ ID NO: 15 or an amino acid sequence having at least 80%, at least 85%, at least 90%, at least 95%, at least 98%, or at least 99% sequence identity to SEQ ID NO: 15, and the VL1 comprises an amino acid sequence as set forth in SEQ ID NO: 16 or an amino acid sequence having at least 80%, at least 85%, at least 90%, at least 95%, at least 98%, or at least 99% sequence identity to SEQ ID NO: 16.

In some embodiments, the protease is selected from the group consisting of uPA, MMP (e.g., MMP2, MMP9, MMP10, MMP11, MMP12, MMP13, MMP14, MMP15), matriptase B, cathepsin B, cathepsin D, collagenase and stromelysin.

In some embodiments, the protease cleavage site comprises an amino acid sequence selected from any of SEQ ID NOs: 17-20. In some embodiments, the protease cleavage site comprises an amino acid sequence selected from RRXaa, LXaa, AXaa, FXaa or WXaa, wherein Xaa is any amino acid. In some embodiments, the protease cleavage site comprises an amino acid sequence selected from FXaa, YXaa or LXaa, wherein Xaa is any amino acid, preferably is not Ala or Val.

In some embodiments, the first linker comprises two or more protease cleavage sites connected in tandem.

In some embodiments, the first linker comprises an amino acid sequence selected from SEQ ID NOs: 21-30.

In some embodiments, the bispecific antibody disclosed herein comprises:
(i) a first polypeptide chain comprising, from N terminal to C terminal, the VH1, an heavy chain constant region 1 (CH1), an heavy chain constant region 2 (CH2), and an heavy chain constant region 3 (CH3); and
(ii) a second polypeptide chain comprising, from N terminal to C terminal, the VL1, a light chain constant region (CL), an heavy chain constant region 2 (CH2), and an heavy chain constant region 3 (CH3);
wherein the VH1 and/or the VL1, at its N terminal, is linked to the mask peptide via the first linker; and
(1) wherein the second binding region comprises a scFv comprising the VH2 and the VL2, wherein the scFv is linked to the C terminal of the first polypeptide chain and/or the second polypeptide chain via a linker, or wherein the scFv is linked to the N terminal of the VH1 via a linker and the mask peptide is linked to the N terminal of the VL1 via the first linker, or wherein the scFv is linked to the N terminal of the VL1 via a linker and the mask peptide is linked to the N terminal of the VH1 via the first linker; or
(2) wherein the VH2 is linked to the C terminal of the first polypeptide chain via a linker and the VL2 is linked to the C terminal of the second polypeptide chain via a linker, or wherein the VL2 is linked to the C terminal of the first polypeptide chain via a linker and the VH2 is linked to the C terminal of the second polypeptide chain via a linker; or
(3) wherein the VH2 is linked to the C terminal of the VH1 via a linker and the VL2 is linked to the C terminal of the VL1 via a linker, or wherein the VH2 is linked to the C terminal of the VL1 via a linker and the VL2 is linked to the C terminal of the VH1 via a linker.

In some embodiments, the first polypeptide chain comprises, from N terminal to C terminal, VH1-linker-VH2-CH1-CH2-CH3; and the second polypeptide chain comprises, from N terminal to C terminal, VL1-linker-VL2-CL-CH2-CH3.

In some embodiments, the first polypeptide chain comprises, from N terminal to C terminal, VH1-CH1-CH2-CH3-linker-VH2; and the second polypeptide chain comprises, from N terminal to C terminal, VL1-CL-CH2-CH3-linker-VL2.

In some embodiments, the first polypeptide chain comprises, from N terminal to C terminal, VH1-CH1-CH2-CH3; and the second polypeptide chain comprises, from N terminal to C terminal, scFv-linker-VL1-CL-CH2-CH3.

In some embodiments, the first polypeptide chain comprises, from N terminal to C terminal, VH1-CH1-CH2-CH3-linker-scFv; and the second polypeptide chain comprises, from N terminal to C terminal, VL1-CL-CH2-CH3-linker-scFv.

In some embodiments, the first polypeptide chain comprises, from N terminal to C terminal, the mask peptide-the first linker-VH1-linker-VH2-CH1-CH2-CH3; and the second polypeptide chain comprises, from N terminal to C terminal, VL1-linker-VL2-CL-CH2-CH3.

In some embodiments, the first polypeptide chain comprises, from N terminal to C terminal, the mask peptide-the first linker-VH1-CH1-CH2-CH3; and the second polypeptide chain comprises, from N terminal to C terminal, scFv-linker-VL1-CL-CH2-CH3.

In some embodiments, each of the linkers is independently comprise an amino acid sequence selected from GGGSS(GGGGS)n, (GGGS)n and (GGGGS)n, wherein n is an integer selected from 1-4. In preferred embodiments, each of the linkers is independently comprise an amino acid sequence selected from any of SEQ ID NOs: 47-49.

In some embodiments, the TAA is selected from the group consisting of EGFR, EpCAM, HER2, PSMA, gpA33, CD276, CEA, CD19, CD20, CD22, CD30, CD33, CD123, FLT3 and BCMA.

In some embodiments, the TAA is EGFR, and the VH2 comprises HCDRs 1-3 having the amino acid sequences as set forth in SEQ ID NOs: 31-33 respectively, and the VL2 comprises LCDRs 1-3 having the amino acid sequences as set forth in SEQ ID NOs: 34-36 respectively.

In some embodiments, the TAA is EGFR, and the VH2 comprises HCDRs 1-3 having the amino acid sequences as set forth in SEQ ID NOs: 37-39 respectively, and the VL2 comprises LCDRs 1-3 having the amino acid sequences as set forth in SEQ ID NOs: 40-42 respectively.

In some embodiments, the TAA is EGFR, and the VH2 comprises an amino acid sequence as set forth in SEQ ID NO: 43 or an amino acid sequence having at least 80%, at least 85%, at least 90%, at least 95%, at least 98%, or at least 99% sequence identity to SEQ ID NO: 43, and the VL2 comprises an amino acid sequence as set forth in SEQ ID NO: 44 or an amino acid sequence having at least 80%, at least 85%, at least 90%, at least 95%, at least 98%, or at least 99% sequence identity to SEQ ID NO: 44.

In some embodiments, the TAA is EGFR, and the VH2 comprises an amino acid sequence as set forth in SEQ ID NO: 45 or an amino acid sequence having at least 80%, at least 85%, at least 90%, at least 95%, at least 98%, or at least 99% sequence identity to SEQ ID NO: 45, and the VL2 comprises an amino acid sequence as set forth in SEQ ID NO: 46 or an amino acid sequence having at least 80%, at least 85%, at least 90%, at least 95%, at least 98%, or at least 99% sequence identity to SEQ ID NO: 46.

In some embodiments, the second binding region comprises a scFv comprising an amino acid sequence as set forth in SEQ ID NO: 75 or SEQ ID NO: 76 or an amino acid sequence having at least 80%, at least 85%, at least 90%, at least 95%, at least 98%, or at least 99% sequence identity to SEQ ID NO: 75 or SEQ ID NO: 76.

In some embodiments, one or both of the CH3 comprise at least one amino acid mutation that is capable of decreasing homodimerization between the first and second polypeptide chains.

In preferred embodiments, amino acid T366 of the one or both CH3 is substituted with L (Leucine), and amino acid Y407 of the one or both CH3 is substituted with I (Isoleucine), F (Phenylalanine), L (Leucine), M (Methionine), H (Histidine), K (Lysine), S (Serine), Q (Glutamine), T (Threonine), W (Tryptophan), A (Alanine), G (Glycine) or N (Asparagine).

In some embodiments, one or both of the CH2 comprise at least one amino acid mutation that is capable of decreasing the effector function of the bispecific antibody, preferably the at least one mutation is selected from L234A, L235A, G237A, or any combination thereof.

In some embodiments, the bispecific antibody disclosed herein comprises:
a first polypeptide chain comprising an amino acid as set forth in SEQ ID NO: 51 or an amino acid sequence having at least 80%, at least 85%, at least 90%, at least 95%, at least 98%, or at least 99% sequence identity to SEQ ID NO: 51, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 52 or an amino acid sequence having at least 80%, at least 85%, at least 90%, at least 95%, at least 98%, or at least 99% sequence identity to SEQ ID NO: 52, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 53 or an amino acid sequence having at least 80%, at least 85%, at least 90%, at least 95%, at least 98%, or at least 99% sequence identity to SEQ ID NO: 53, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 54 or an amino acid sequence having at least 80%, at least 85%, at least 90%, at least 95%, at least 98%, or at least 99% sequence identity to SEQ ID NO: 54, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 55 or an amino acid sequence having at least 80%, at least 85%, at least 90%, at least 95%, at least 98%, or at least 99% sequence identity to SEQ ID NO: 55, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 56 or an amino acid sequence having at least 80%, at least 85%, at least 90%, at least 95%, at least 98%, or at least 99% sequence identity to SEQ ID NO: 56, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 52 or an amino acid sequence having at least 80%, at least 85%, at least 90%, at least 95%, at least 98%, or at least 99% sequence identity to SEQ ID NO: 52, and a second polypeptide chain comprising an amino acid as set forth in SEQ ID NO: 57 or an amino acid sequence having at least 80%, at least 85%, at least 90%, at least 95%, at least 98%, or at least 99% sequence identity to SEQ ID NO: 57; or
a first polypeptide chain comprising an amino acid as set forth in SEQ ID NO: 58 or an amino acid sequence having at least 80%, at least 85%, at least 90%, at least 95%, at least 98%, or at least 99% sequence identity to SEQ ID NO: 58, and a second polypeptide chain comprising an amino acid as set forth in SEQ ID NO: 59 or an amino acid sequence having at least 80%, at least 85%, at least 90%, at least 95%, at least 98%, or at least 99% sequence identity to SEQ ID NO: 59; or
a first polypeptide chain comprising an amino acid as set forth in SEQ ID NO: 60 or an amino acid sequence having at least 80%, at least 85%, at least 90%, at least 95%, at least 98%, or at least 99% sequence identity to SEQ ID NO: 60, and a second polypeptide chain comprising an amino acid as set forth in SEQ ID NO: 61 or an amino acid sequence having at least 80%, at least 85%, at least 90%, at least 95%, at least 98%, or at least 99% sequence identity to SEQ ID NO: 61; or
a first polypeptide chain comprising an amino acid as set forth in SEQ ID NO: 62 or an amino acid sequence having at least 80%, at least 85%, at least 90%, at least 95%, at least 98%, or at least 99% sequence identity to SEQ ID NO: 62, and a second polypeptide chain comprising an amino acid as set forth in SEQ ID NO: 63 or an amino acid sequence having at least 80%, at least 85%, at least 90%, at least 95%, at least 98%, or at least 99% sequence identity to SEQ ID NO: 63; or
a first polypeptide chain comprising an amino acid as set forth in SEQ ID NO: 64 or an amino acid sequence having at least 80%, at least 85%, at least 90%, at least 95%, at least 98%, or at least 99% sequence identity to SEQ ID NO: 64, and a second polypeptide chain comprising an amino acid as set forth in SEQ ID NO: 65 or an amino acid sequence having at least 80%, at least 85%, at least 90%, at least 95%, at least 98%, or at least 99% sequence identity to SEQ ID NO: 65; or
a first polypeptide chain comprising an amino acid as set forth in SEQ ID NO: 66 or an amino acid sequence having at least 80%, at least 85%, at least 90%, at least 95%, at least 98%, or at least 99% sequence identity to SEQ ID NO: 66, and a second polypeptide chain comprising an amino acid as set forth in SEQ ID NO: 67 or an amino acid sequence having at least 80%, at least 85%, at least 90%, at least 95%, at least 98%, or at least 99% sequence identity to SEQ ID NO: 67; or
a first polypeptide chain comprising an amino acid as set forth in SEQ ID NO: 66 or an amino acid sequence having at least 80%, at least 85%, at least 90%, at least 95%, at least 98%, or at least 99% sequence identity to SEQ ID NO: 66, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 69 or an amino acid sequence having at least 80%, at least 85%, at least 90%, at least 95%, at least 98%, or at least 99% sequence identity to SEQ ID NO: 69, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 70 or an amino acid sequence having at least 80%, at least 85%, at least 90%, at least 95%, at least 98%, or at least 99% sequence identity to SEQ ID NO: 70, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 71 or an amino acid sequence having at least 80%, at least 85%, at least 90%, at least 95%, at least 98%, or at least 99% sequence identity to SEQ ID NO: 71, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 72 or an amino acid sequence having at least 80%, at least 85%, at least 90%, at least 95%, at least 98%, or at least 99% sequence identity to SEQ ID NO: 72, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 73 or an amino acid sequence having at least 80%, at least 85%, at least 90%, at least 95%, at least 98%, or at least 99% sequence identity to SEQ ID NO: 73, and a second polypeptide chain comprising an amino acid as set forth in SEQ ID NO: 74 or an amino acid sequence having at least 80%, at least 85%, at least 90%, at least 95%, at least 98%, or at least 99% sequence identity to SEQ ID NO: 74; or
a first polypeptide chain comprising an amino acid as set forth in SEQ ID NO: 77 or an amino acid sequence having at least 80%, at least 85%, at least 90%, at least 95%, at least 98%, or at least 99% sequence identity to SEQ ID NO: 77, and a second polypeptide chain comprising an amino acid as set forth in SEQ ID NO: 78 or an amino acid sequence having at least 80%, at least 85%, at least 90%, at least 95%, at least 98%, or at least 99% sequence identity to SEQ ID NO: 78.

In another aspect, the present invention provides a nucleic acid comprising a nucleotide sequence encoding the bispecific antibody disclosed herein.

In still another aspect, the present invention provides a vector comprising the nucleic acid disclosed herein.

In yet another aspect, the present invention provides a host cell comprising the nucleic acid or the vector disclosed herein.

In still another aspect, the present invention provides a pharmaceutical composition comprising (i) the bispecific antibody disclosed herein; and (ii) a pharmaceutically acceptable carrier or excipient.

In some embodiments of the pharmaceutical composition disclosed herein, the pharmaceutical composition further comprises a second therapeutic agent.

In some embodiments, the second therapeutic agent is selected from an antibody, a chemotherapeutic agent and a small molecule drug.

In some embodiments, the second therapeutic agent is selected from a Bruton's tyrosine kinase (BTK) inhibitor, a PI3K inhibitor, a HDAC inhibitor, an ERK inhibitor, a MAPK inhibitor, a PD-1/PD-L1 inhibitor, a LAG3 inhibitor, a CTLA-4 inhibitor, a TIGIT inhibitor, a TIM3 inhibitor, or glucocorticoid.

In yet another aspect, the present invention provides a conjugate comprising the bispecific antibody disclosed herein and a chemical moiety conjugated thereto.

In some embodiments of the conjugate disclosed herein, the chemical moiety is selected from the group consisting of a therapeutic agent, a detectable moiety, and an immune stimulatory molecule.

In still another aspect, the present invention provides a method of treating a cancer in a subject, comprising administering to the subject an effective amount of the bispecific antibody disclosed herein, the pharmaceutical composition disclosed herein, or the conjugate disclosed herein.

In some embodiments of the method disclosed herein, the TAA is EGFR and the cancer is an EGFR positive cancer.

In some embodiments, the cancer is selected from the group consisting of fibrosarcoma, colon cancer, colorectal cancer, liver cancer, kidney cancer, lung cancer, gastric cancer, ovarian cancer, breast cancer, pancreatic cancer, head and neck cancer, glioblastoma, and prostatic cancer.

In some embodiments, the method further comprises administering to the subject a second therapeutic agent.

In some embodiments, the second therapeutic agent is selected from an antibody, a chemotherapeutic agent and a small molecule drug.

In some embodiments, the second therapeutic agent is selected from a Bruton's tyrosine kinase (BTK) inhibitor, a PI3K inhibitor, a HDAC inhibitor, an ERK inhibitor, a MAPK inhibitor, a PD-1/PD-L1 inhibitor, a LAG3 inhibitor, a CTLA-4 inhibitor, a TIGIT inhibitor, a TIM3 inhibitor, and glucocorticoid.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows schematic representation of controls of bispecific antibodies of the present invention. 1.1-Unmask HBITE: CD3×EGFR bispecific antibody without protease cleavage site; 1.4-Noncleavable iBiTE: bispecific antibody with a mutant protease cleavage site and two mask peptides in tandem. Stars denote mutations which decrease or abrogate Fc homodimerization. Exemplary amino acid sequences of the mask peptide are shown in FIG. 1.

FIG. 2 shows schematic representation of examples of an embodiment of protease cleavable bispecific antibodies of the present invention. 1.1/1.2/1.3 proBiTEs: comprising a linker with a protease cleavage site and different mask peptides; 1.4/1.4.1 proBiTEs: comprising a linker with a protease cleavage site and two mask peptides in tandem; 1.4.3 proBiTE: comprising a linker whose length between Mask1 and protease cleavage site is shorter than 1.4 proBiTE. Stars denote mutations which decrease or abrogate Fc homodimerization. All unlabeled modules have the same meanings as in FIG. 1.

FIG. 3 shows schematic representation of examples of an embodiment of protease cleavable bispecific antibodies of the present invention. 1.5 proBiTE: both light and heavy chains contain a uPA protease cleavage site; 1.6 proBiTE: comprising a linker with a protease cleavage site and two mask peptides in tandem (Mask1+1); 1.6.1 proBiTE: the heavy chain comprises two mask peptides in tandem (Mask1+2), while the light chain comprises a Single-Chain Fragment Variable (scFv) of anti-EGFR antibody fused to the N terminus of the VL-CL via a (G₄S)₄ linker; 1.7 proBiTE: both light and heavy chains contain anti-EGFR scFv which is fused to the C terminus of the IgG1 mFc via a (G₄S)₃ linker; 1.7.1 proBiTE: both light and heavy chains contain anti-EGFR scFv which is fused to the C terminus of the IgG1 mFc via a (G₄S)₄ linker. Stars denote mutations which decrease or abrogate Fc homodimerization. All unlabeled modules have the same meanings as in FIG. 1.

FIG. 4 shows mechanism of action of the bispecific antibodies of the present invention in the absence or presence of tumor-associated proteases. Stars denote mutations which decrease or abrogate Fc homodimerization. All unlabeled modules have the same meanings as in FIG. 1.

FIG. 5 shows mechanism of action of the bispecific antibodies of the present invention in circulation (left panel) and in tumor tissue (right panel). Stars denote mutations which decrease or abrogate Fc homodimerization. All unlabeled modules have the same meanings as in FIG. 1.

FIGS. 6A-6C shows expression and purification of proBiTEs of the invention.

FIG. 6D shows binding affinity of proBiTEs 1.1-1.5 against human antigen CD3 in the absence or presence of uPA, as measured by ELISA.

FIG. 7 shows binding affinity of 1.4.3 proBiTE against human antigen CD3 in the absence or presence of uPA, as measured by ELISA.

FIG. 8A shows binding affinity of 1.6 and 1.7 proBiTEs against human antigen CD3 in the absence or presence of uPA, as measured by ELISA.

FIG. 8B shows binding affinity of 1.6 and 1.7 proBiTEs against human antigen EGFR in the absence or presence of uPA, as measured by ELISA.

FIG. 9A shows binding affinity of 1.6.1 and 1.7.1 proBiTEs against human antigen CD3 in the absence or presence of uPA, as measured by ELISA.

FIG. 9B shows binding affinity of 1.6.1 and 1.7.1 proBiTEs against EGFR in the absence or presence of uPA, as measured by ELISA.

FIG. 10 shows schematic representation of examples of an embodiment of 1.6.1 proBiTE variants of the present invention. 1.6.1M proBiTE: comprising the LALA-G237A mutations (L234A, L235A, and G237A); 1.6.1M-Noncleavable: comprising a mutant protease cleavage site; 1.6.1M-Unmask: without a protease cleavage site. Stars denote mutations which decrease or abrogate Fc homodimerization. All unlabeled modules have the same meanings as in FIG. 1.

FIG. 11 shows schematic representation of examples of an embodiment of 1.6.1M-Pa proBiTE and its variants of the present invention. 1.6.1M-Pa proBiTE: the fragment of anti-EGFR is derived from Panitumumab domain. 1.6.1M-2-1-Pa/1.6.1M-2-2-Pa/1.6.1M-2-3-Pa/1.6.1M-2-4-Pa proBiTEs: the N-terminus of the anti-CD3 Fab of the heavy chain is connected with two protease cleavage sites in tandem.

FIG. 12 shows schematic representation of examples of an embodiment of 1.4.3 proBiTE variants of the present invention. 1.4.3-Pa proBiTE: the fragment of anti-EGFR is derived from Panitumumab domain; 1.4.3M-Pa proBiTE: comprising the LALA-G237A mutations (L234A, L235A, and G237A) and the fragment of anti-EGFR is derived from Panitumumab domain.

FIG. 13 shows binding affinity of 1.6.1M proBiTE against human antigen CD3, as measured by ELISA.

FIG. 14 shows binding affinity of 1.6.1M-Pa proBiTE against human antigen CD3 and EGFR in the absence or presence of uPA, as measured by ELISA.

FIG. 15A shows binding affinity of 1.6.1M-2-1-Pa proBiTE against human antigen CD3 and EGFR in the absence of uPA, as measured by ELISA.

FIG. 15B shows binding affinity of 1.6.1M-2-1-Pa proBiTE against human antigen CD3 and EGFR in the presence of uPA, as measured by ELISA.

FIG. 16A shows binding affinity of 1.4.3-Pa and 1.4.3M-Pa proBiTEs against human antigen CD3 in the absence or presence of uPA, as measured by ELISA.

FIG. 16B shows binding affinity of 1.4.3-Pa and 1.4.3M-Pa proBiTEs against human antigen EGFR in the absence or presence of uPA, as measured by ELISA.

FIG. 17 shows content of human uPA in various tumor cell lines, as measured by ELISA.

FIG. 18 shows killing of HT1080 cells by 1.6.1M-Pa and 1.6.1M-2-1-Pa proBiTE in the presence of human PBMCs. Target cells (HT1080) and effector cells (PBMC) are at a ratio of 1:10. 1.6.1M-Pa-Noncleavable and 1.6.1M-Pa-Unmask serve as control.

FIG. 19 shows killing of MC38-EGFR cells by 1.6.1M-Pa and 1.6.1M-2-1-Pa proBiTEs in the presence of human PBMCs. Target cells (MC38-EGFR) and effector cells (PBMC) are at a ratio of 1:10. 1.6.1M-Pa-Noncleavable and 1.6.1M-Pa-Unmask serve as control.

FIG. 20 shows killing of HT1080 cells by 1.4.3-Pa proBiTE in the presence of human PBMCs. Target cells (HT1080) and effector cells (PBMC) are at a ratio of 1:10.

FIG. 21 shows killing of MC38-EGFR cells by 1.4.3-Pa proBiTE in the presence of human PBMCs. Target cells (MC38-EGFR) and effector cells (PBMC) are at a ratio of 1:10.

FIG. 22 shows inhibition of tumor growth in B-NDG mice by 1.6.1M-Pa proBiTE.

FIG. 23 shows inhibition of tumor growth in B-NDG mice by 1.6.1M-2-1-Pa proBiTE.

FIG. 24 shows inhibition of tumor growth in B-NDG mice by 1.4.3M-Pa proBiTE.

### DETAILED DESCRIPTION OF THE INVENTION

The aforementioned features and advantages of the invention as well as additional features and advantages thereof will be more clearly understood hereafter as a result of a detailed description of the following embodiments when taken in conjunction with the drawings.

The embodiments described herein with reference to drawings are explanatory, illustrative, and used to generally understand the present invention. The embodiments shall not be construed to limit the scope of the present invention. The same or similar elements and the elements having same or similar functions are denoted by like reference numerals throughout the descriptions.

Unless indicated or defined otherwise, all terms used have their usual meaning in the art, which will be clear to the skilled person. Reference is for example made to the standard handbooks, such as Leuenberger, H. G. W, Nagel, B. and Klbl, H. eds., “A multilingual glossary of biotechnological terms: (IUPAC Recommendations)”, Helvetica Chimica Acta (1995), CH-4010 Basel, Switzerland; Sambrook et al, “Molecular Cloning: A Laboratory Manual” (2nd Ed.), Vols. 1-3, Cold Spring Harbor Laboratory Press (1989); F. Ausubel et al, eds., “Current protocols in molecular biology”, Green Publishing and Wiley InterScience, New York (1987); Roitt et al., “Immunology (6th Ed.), Mosby/Elsevier, Edinburgh (2001); and Janeway et al., “Immunobiology” (6th Ed.), Garland Science Publishing/Churchill Livingstone, New York (2005), as well as the general background art cited above.

As used herein, singular forms “a”, “and,” and “the” include plural referents unless the context clearly indicates otherwise. Thus, for example, reference to “an antibody” includes a plurality of antibodies and reference to “an antibody” in some embodiments includes multiple antibodies, and so forth.

Unless indicated or defined otherwise, the term “comprise”, and variations such as “comprises” and “comprising”, should be understood to imply the inclusion of a stated elements or step or group of elements or steps but not the exclusion of any other element or step or group of elements or steps.

As used herein, the term “antibody” refers to an immunoglobulin molecule which has the ability to specifically bind to a specific antigen. An antibody often comprises a variable region and a constant region in each of a heavy chain and a light chain. The variable regions of the heavy and light chains of antibodies contain a binding domain that interacts with an antigen. The constant regions of antibodies may mediate the binding of the immunoglobulin to host tissues or factors, including various cells of the immune system (such as effector cells) and components of the complement system such as C1q, the first component in the classical pathway of complement activation. Accordingly, most antibodies have a heavy chain variable region (VH) and a light chain variable region (VL) that together form the portion of the antibody that binds to the antigen.

A “light chain variable region” (VL) or “heavy chain variable region” (VH) consists of a “framework” region interrupted by three “complementarity determining regions” or “CDRs”. The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as LCDR1, LCDR2, and LCDR3; CDRs 1, 2, and 3 of a VH domain are also referred to herein, respectively, as HCDR1, HCDR2, and HCDR3.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-HI is a composite of Chothia and Kabat CDRs; the AbM definition used by Oxford Molecular's antibody modelling software; and the CONTACT definition of Martin et al. (world wide web bioinfo.org.uk/abs). Kabat provides a widely used numbering convention (Kabat numbering system) in which corresponding residues between different heavy chains or between different light chains are assigned the same number. The present disclosure can use CDRs defined according to any of these numbering systems, although preferred embodiments use Kabat defined CDRs.

The term “antibody” as used herein should be understood in its broadest meaning, and includes monoclonal antibodies (including full-length monoclonal antibodies), polyclonal antibodies, antibody fragments, and multi-specific antibodies containing at least two different antigen binding regions (e.g., bispecific antibodies). The antibody may contain additional modifications, such as non-naturally occurring amino acids, mutations in Fc regions, and mutations in glycosylation sites. Antibodies also include post-translation modified antibodies, fusion proteins containing the antigenic determinants of the antibody, and immunoglobulin molecules containing any other modifications to antigen recognition sites, as long as these antibodies exhibit desired biological activity.

The term “bispecific antibody” is in the context of the present invention to be understood as an antibody having two different antigen-binding regions defined by different antibody sequences. This can be understood as different target binding but includes as well binding to different epitopes in one target.

Antigen binding fragments of an antibody refer to one or more fragments of an antibody that retain the ability to specifically bind to an antigen. It has been shown that the antigen binding function of an antibody can be performed by fragments of a full-length antibody.

Examples of antigen binding fragments encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fab′ fragment, which is essentially an Fab with part of the hinge region; (iv) a Fd fragment consisting of the VH and CH1 domains; (v) a Fd′ fragment having VH and CH1 domains and one or more cysteine residues at the C-terminus of the CH1 domain; (vi) a Fv fragment consisting of the VL and VH domains of a single arm of an antibody, (vii) a dAb fragment, which consists of a VH domain; (viii) an isolated complementarity determining region (CDR); and (ix) a nanobody, a heavy chain variable region containing a single variable domain and two constant domains. Furthermore, although the two domains of the Fv fragment, VL and VH are coded for by separate genes, they can be joined, using recombinant methods, by a synthetic linker that enables them to be made as a single protein chain in which the VL and VH regions pair to form monovalent molecules (known as single chain Fv (scFv)). Such single chain antibodies are also intended to be encompassed within the term “antigen binding fragment” of an antibody. Furthermore, the term also includes a “linear antibody” comprising a pair of tandem Fd segments (VH-CH1-VH-CH1), which forms an antigen binding region together with a complementary light chain polypeptide, and a modified version of any of the foregoing fragments, which retains antigen binding activity.

These antigen binding fragments can be obtained using conventional techniques known to those with skill in the art, and the fragments are screened for utility in the same manner as are intact antibodies.

As used herein, the term “bind” or “specifically bind” refers to a non-random binding reaction between two molecules, such as between an antibody and its target antigen. The binding specificity of an antibody can be determined based on affinity and/or avidity. The affinity, represented by the equilibrium constant for the dissociation of an antigen with an antibody (KD), is a measure for the binding strength between an antigenic determinant and an antigen-binding site on the antibody: the lesser the value of the KD, the stronger the binding strength between an antigenic determinant and the antibody. Alternatively, the affinity can also be expressed as the affinity constant (KA), which is 1/KD.

Avidity is the measure of the strength of binding between an antibody and the pertinent antigen. Avidity is related to both the affinity between an antigenic determinant and its antigen binding site on the antibody and the number of pertinent binding sites present on the antibody. Typically, an antibody will bind to an antigen with a dissociation constant (KD) of 10⁻⁵ to 10⁻¹² M or less, and preferably 10⁻⁷ to 10⁻¹² M or less and more preferably 10⁻⁸ to 10⁻¹² M, and/or with a binding affinity of at least 10⁷ M⁻¹, preferably at least 10⁸ M⁻¹, more preferably at least 10⁹ M⁻¹, such as at least 10¹² M⁻¹. Any K_{D} value greater than 10⁻⁴ M is generally considered to indicate non-specific binding. Specifically binding of an antibody to an antigen or antigenic determinant can be determined in any suitable manner known, including, for example, Scatchard analysis and/or competitive binding assays, such as radioimmunoassays (RIA), enzyme immunoassays (EIA) and sandwich competition assays, and the different variants thereof known in the art.

As used herein, the term “CD3” refers to the human CD3 protein complex, which has five peptide chains, γ chain, δ chain, ε chain, ζ chain and η chain, and is associated with the T cell receptor α and β chains to form a TCR-CD3 complex. The term includes any CD3 variants, isoforms and species homologs which are naturally expressed by cells, including T cells, or are expressed on cells transfected with genes or cDNA encoding the aforementioned chains.

As used herein, the term “tumor associated antigen” or “TAA” refers to an antigen that is differentially expressed in cancer cells compared to normal cells, and therefore can be used to target cancer cells.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. The epitope defines the smallest binding site of an antibody and therefore is the specific target of the antibody or antigen binding fragment thereof.

The term “epitope of CD3” refers to a site on CD3 molecule to which an anti-CD3 antibody binds.

As used herein, the term “protease expressed or overexpressed in tumor microenvironment” is a tumor-associated protease that is differentially expressed in tumor microenvironment compared to normal tissues.

Urokinase-type plasminogen activator (uPA) is a serine protease involved in tissue remodeling and cell migration. The active form of uPA is bound to its high affinity receptor on the cell surface, where specific inhibitors modulate its enzymatic activity. Matrix metalloproteinases (MMPs) play key roles in metastasis and are associated with survival in various cancers. Membrane-associated serine protease matriptase is widely expressed by epithelial/carcinoma cells. Cathepsin B is a lysosomal cysteine protease of the papain family. Cathepsin D is a lysosomal ‘aspartic proteinase’ and may be one of the more primitive members of the family. Collagenase is a member of the matrix metalloproteinase (MMP) family of enzymes, and is the principal enzyme involved with collagen degradation. Stromelysin, a member of the metalloproteinase gene family, may play a crucial role in pathological turnover of connective tissues.

As used herein, the term “sequence identity” refers to the extent to which two sequences (amino acid) have the same residue at the same positions in an alignment. For example, “an amino acid sequence is X % identical to SEQ ID NO: Y” refers to % identity of the amino acid sequence to SEQ ID NO:Y and is elaborated as X % of residues in the amino acid sequence are identical to the residues of sequence disclosed in SEQ ID NO: Y.

Generally, computer programs are employed for such calculations. Exemplary programs that compare and align pairs of sequences, include ALIGN (Myers and Miller, 1988), FASTA (Pearson and Lipman, 1988; Pearson, 1990) and gapped BLAST (Altschul et al., 1997), BLASTP, BLASTN, or GCG (Devereux et al., 1984).

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide.

Such conservative substitutions preferably are substitutions in which one amino acid within the following groups (a)-(c) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, He, Val and Cys; and (c) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

As used herein, the term “vector” is intended to refer to a nucleic acid molecule capable of transporting another nucleic acid to which it has been linked.

As used herein, the term “host cell” refers to a cell into which an expression vector has been introduced.

The term “pharmaceutically acceptable” means that the carrier or adjuvant is compatible with the other ingredients of the composition and not substantially deleterious to the recipient thereof and/or that such carrier or adjuvant is approved or approvable for inclusion in a pharmaceutical composition for parenteral administration to humans.

As used herein, the terms “treatment”, “treating”, “treat”, and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment”, as used herein, may include treatment of a disease or disorder (e.g. cancer) in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease); (b) inhibiting the disease, i.e., arresting its development; and (c) relieving the disease, i.e., causing regression of the disease. Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the antibodies or compositions or conjugates disclosed herein to prevent or delay, to alleviate, or to arrest or inhibit development of the symptoms or conditions associated with diseases (e.g. cancers). The term “therapeutic effect” refers to the reduction, elimination, or prevention of the disease, symptoms of the disease, or side effects of the disease in the subject.

The term “effective amount” as used herein means the amount that, when administered to a subject for treating a disease, is sufficient to effect treatment for that disease.

The term “subject”, as used herein, refers to any mammalian subject for whom diagnosis, treatment or therapy is desired. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc.

### ABBREVIATIONS

As used herein, the term “BiTE” refers to “bispecific T-cell engager”, which is a polypeptide molecule having two antigen-binding domains, one of which binds to a T-cell antigen and the second of which binds to an antigen present on the surface of target cells. Thus, the BiTE of the disclosure has an antigen binding region that binds to TAA and a second antigen binding region that is directed towards a T-cell antigen, e.g., CD3.

As used herein, “proBiTE” refers to protease cleavable bispecific T cell engager. The examples of the proBiTEs of the present invention include 1.1, 1.2, 1.3, 1.4, 1.4.1, 1.4.3, 1.5, 1.6, 1.6.1, 1.7, 1.7.1, 1.6.1M, 1.6.1M-Pa, 1.6.1M-2-1-Pa, 1.6.1M-2-2-Pa, 1.6.1M-2-3-Pa, 1.6.1M-2-4-Pa, 1.4.3-Pa, and 1.4.3M-Pa proBiTEs, in which 1.6.1M, 1.6.1M-Pa, 1.6.1M-2-1-Pa, 1.6.1M-2-2-Pa, 1.6.1M-2-3-Pa and 1.6.1M-2-4-Pa proBiTEs are variants of 1.6.1 proBiTE, and 1.4.3-Pa and 1.4.3M-Pa proBiTEs are variants of 1.4.3 proBiTE.

“1.1 proBiTE” contains a linker with a uPA protease cleavage site

(SEQ ID NO: 21
(GGGSGGGSGGGSGLSGRSDNHGGS,

double underlined is the uPA protease cleavage site)) and a mask peptide (QDGNEEMGGI (SEQ ID NO: 3, Mask2)).

“1.2 proBiTE” contains a linker with a uPA protease cleavage site

(SEQ ID NO: 21
(GGGSGGGSGGGSGLSGRSDNHGGS,

double underlined is the uPA protease cleavage site)) and a mask peptide (QDGNEEMGGITPY (SEQ ID NO: 2, Mask3)).

“1.3 proBiTE” contains a linker with a uPA protease cleavage site

(SEQ ID NO: 21
(GGGSGGGSGGGSGLSGRSDNHGGS,

double underlined is the uPA protease cleavage site)) and a mask peptide (QDGNEEMGGITQTPYKVSISGT (SEQ ID NO: 1, Mask4)).

“1.4 proBiTE” contains a linker with a uPA protease cleavage site

(SEQ ID NO: 22
(GGGGGSGGGSGGGSGLSGRSDNHGGS,

double underlined is the uPA protease protease cleavage site)) and a mask peptide (QDGNEEMGGIGGGSQDGNEEM (SEQ ID NO: 6, Mask2+1)).

“1.4.1 proBiTE” contains a linker with a uPA protease cleavage site

(SEQ ID NO: 21
(GGGSGGGSGGGSGLSGRSDNHGGS,

double underlined is the uPA protease cleavage site)) and a mask peptide (QDGNEEMGGGSQDGNEEMGGITQTPY (SEQ ID NO: 7, Mask1+3)).

“1.4.3 proBiTE” contains a linker with a uPA protease cleavage site

(SEQ ID NO: 23
(GGGGGSGGGSGLSGRSDNHGGS,

double underlined is the uPA protease cleavage site)) and a mask peptide (QDGNEEMGGIGGGSQDGNEEM (SEQ ID NO: 6, Mask2+1)).

“1.5 proBiTE” contains a linker with a uPA protease cleavage site

(SEQ ID NO: 21
(GGGSGGGSGGGSGLSGRSDNHGGS,

double underlined is the uPA protease cleavage site)) and a mask peptide (QDGNEEMGGITPY (SEQ ID NO: 2, Mask3)) in heavy chain and a linker with a uPA protease cleavage site

(SEQ ID NO: 21
(GGGSGGGSGGGSGLSGRSDNHGGS,

double underlined is the uPA protease cleavage site)) and a mask peptide (QDGNEEMGGITPY (SEQ ID NO: 2, Mask3)) in light chain.

“1.6 proBiTE” contains a linker with a uPA protease cleavage site

(SEQ ID NO: 24
(GGGGSGGGSGGGSGLSGRSDNHGGSSGGS,

double underlined is the uPA protease cleavage site)) and a mask peptide (QDGNEEMGGGSQDGNEEM (SEQ ID NO: 5, Mask1+1)).

“1.6.1 proBiTE” contains a linker with a uPA protease cleavage site

(SEQ ID NO: 25
(GGGSGGGGSGLSGRSDNHGGGGS,

double underlined is the uPA protease cleavage site)) and a mask peptide (QDGNEEMGGGSQDGNEEMGGI (SEQ ID NO: 8, Mask1+2)) in heavy chain and an anti-TAA scFv in light chain.

“1.7 proBiTE” contains a linker with a uPA protease cleavage site

(SEQ ID NO: 24
(GGGGSGGGSGGGSGLSGRSDNHGGSSGGS,

double underlined is the uPA protease cleavage site)), a mask peptide (QDGNEEMGGGSQDGNEEM (SEQ ID NO: 5, Mask1+1)) and an anti-TAA scFv in heavy chain, and an anti-TAA scFv in light chain.

“1.7.1 proBiTE” contains a linker with a uPA protease cleavage site

(SEQ ID NO: 26
(GGGGSGGGSGGGSGLSGRSDNHGGGGS,

double underlined is the uPA protease cleavage site)), a mask peptide (QDGNEEMGGGSQDGNEEM (SEQ ID NO: 5, Mask1+1)) and an anti-TAA scFv in heavy chain, and an anti-TAA scFv in light chain.

“1.6.1M proBiTE” is a variant of 1.6.1 proBiTE and contains AAGA mutation (L234A, L235A, and G237A) in both heavy chain and light chain.

“1.6.1M-Pa proBiTE” is a variant of 1.6.1 proBiTE and contains AAGA mutation (L234A, L235A, and G237A) in both heavy chain and light chain and an anti-EGFR panitumumab scFv in light chain.

“1.6.1M-2-1-Pa proBiTE” is a variant of 1.6.1 M-Pa proBiTE and contains a linker with two uPA protease cleavage sites in tandem

(SEQ ID NO: 27
(GGGSGGGGSGLSGRSDNHGGLSGRSDNHGGS,

double underlined is the uPA protease cleavage site)) and a mask peptide (QDGNEEMGGGSQDGNEEMGGI (SEQ ID NO: 8, Mask1+2)) in heavy chain.

“1.6.1M-2-2-Pa proBiTE” is a variant of 1.6.1 M-Pa proBiTE and contains a linker with a uPA protease cleavage site and a MMP2 protease cleavage site in tandem

(SEQ ID NO: 28
(GGGSGGGGSGLSGRSDNHGGPLGLAGGGS,

double underlined is the uPA protease cleavage site and bolded is the MMP2 protease cleavage site)) and a mask peptide (QDGNEEMGGGSQDGNEEMGGI (SEQ ID NO: 8, Mask1+2)) in heavy chain.

“1.6.1M-2-3-Pa proBiTE” is a variant of 1.6.1 M-Pa proBiTE and contains a linker with a uPA protease cleavage site and a matriptase B (MatB) protease cleavage site in tandem

(SEQ ID NO: 29
(GGGSGGGGSGLSGRSDNHGGPMAKKGGS,

double underlined is the uPA protease cleavage site and bolded is the MatB protease cleavage site)) and a mask peptide (QDGNEEMGGGSQDGNEEMGGI (SEQ ID NO: 8, Mask1+2)) in heavy chain.

“1.6.1M-2-4-Pa proBiTE” is a variant of 1.6.1 M-Pa proBiTE and contains a linker with a uPA protease cleavage site and a MMP9 protease cleavage site in tandem

(SEQ ID NO: 30
(GGGSGGGGSGLSGRSDNHGGPGPAGMKGLPGGS,

double underlined is the uPA protease cleavage site and bolded is the MMP9 protease cleavage site)) and a mask peptide (QDGNEEMGGGSQDGNEEMGGI (SEQ ID NO: 8, Mask1+2)) in heavy chain.

“1.4.3-Pa proBiTE” is a variant of 1.4.3 proBiTE and contains an anti-EGFR panitumumab Fv in replacement of the anti-EFGR Fv of 1.4.3 proBiTE.

“1.4.3M proBiTE” is a variant of 1.4.3 proBiTE and contains AAGA mutation (L234A, L235A, and G237A) in both heavy chain and light chain.

“1.4.3M-Pa proBiTE” is a variant of 1.4.3 proBiTE and contains AAGA mutation (L234A, L235A, and G237A) in both heavy chain and light chain and an anti-EGFR panitumumab Fv in replacement of the anti-EFGR Fv of 1.4.3 proBiTE.

As used herein, “HBiTE” refers to H-shaped bispecific T cell engager. “1.1-unmask” and “1.4.3M-Pa-unmask” are examples of HBiTE.

As used herein, “iBiTE” refers to i-shaped bispecific T cell engager. “1.4-Noncleavable”, “1.4.3-Pa-Noncleavable” and “1.4.3M-Pa-Noncleavable” are examples of iBiTE.

“1.1-unmask” refers to 1.1-unmask HBiTE, which contains neither a protease cleavable site nor a mask peptide.

“1.4-Noncleavable” refers to 1.4-Noncleavable iBITE, which contains a mutant protease cleavage site that is not cleavable by the protease and a mask peptide (QDGNEEMGGIGGGSQDGNEEM (SEQ ID NO: 6, Mask2+1)).

“1.6.1M-unmask” refers to 1.6.1M-unmask BiTE, which is a variant of 1.6.1M proBiTE and contains neither a protease cleavable site nor a mask peptide.

“1.6.1M-Noncleavable” refers to 1.6.1M-Noncleavable BiTE, which is a variant of 1.6.1M proBiTE and contains a mutant protease cleavage site that is not cleavable by the protease and a mask peptide (QDGNEEMGGGSQDGNEEMGGI (SEQ ID NO: 8, Mask1+2)).

“1.6.1M-Pa-unmask” refers to 1.6.1M-Pa-unmask BiTE, which is a variant of 1.6.1M-Pa proBiTE and contains neither a protease cleavable site nor a mask peptide.

“1.6.1M-Pa-Noncleavable” refers to 1.6.1M-Pa-Noncleavable BiTE, which is a variant of 1.6.1M-Pa proBiTE and contains a mutant protease cleavage site that is not cleavable by the protease and a mask peptide (QDGNEEMGGGSQDGNEEMGGI (SEQ ID NO: 8, Mask1+2)).

“1.4.3-Pa-Noncleavable” refers to 1.4.3-Pa-Noncleavable iBiTE, which is a variant of 1.4.3-Pa proBiTE and contains a mutant protease cleavage site that is not cleavable by the protease and a mask peptide (QDGNEEMGGIGGGSQDGNEEM (SEQ ID NO: 6, Mask2+1)).

“1.4.3M-Pa-unmask” refers to 1.4.3M-Pa-unmask HBITE, which is a variant of 1.4.3M-Pa proBiTE and contains neither a protease cleavable site nor a mask peptide.

“1.4.3M-Pa-Noncleavable” refers to 1.4.3M-Pa-Noncleavable iBiTE, which is a variant of 1.4.3M-Pa proBiTE and contains a mutant protease cleavage site that is not cleavable by the protease and a mask peptide (QDGNEEMGGIGGGSQDGNEEM (SEQ ID NO: 6, Mask2+1)).

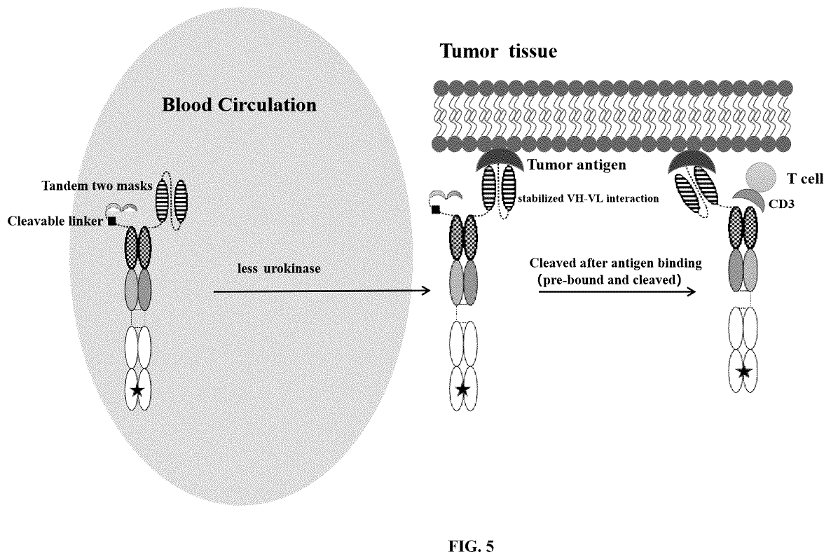

A mechanism of action of the proBiTEs disclosed herein is proposed as follows: in the tumor microenvironment where protease expression is up-regulated, efficient cleavage of the polypeptide linker by the proteases removes the steric hindrance allowing proBiTEs to bind to the CD3 antigen and exert its functions (FIG. 5). In some design of proBiTEs, mask gene fragments are fused to the N terminus of the VH domain of the Fab of a CD3 antibody via a protease non-cleavable and cleavable polypeptide linker, respectively. In tumor tissues, however, proBiTEs bound to tumor antigens could be stabilized due to cooperation between the VH-VL and antibody-antigen interface, leading to retained full function of the bispecific antibodies even after cleavage by proteases (FIG. 5, right panel). It is therefore hypothesized that this unique proBiTE design could further decrease on-target toxicity of this class of bispecific antibodies. In summary, as compared to published monovalent bispecific antibodies developed in the art, the bispecific antibodies of the present invention have the distinctive features of using (i) monomeric Fc domain, for example, human IgG1 Fc, to extend in vivo half-life, and (ii) only two polypeptide chains and employ both Fab and Fv or scFv for heterodimerization and antigen recognition. A combination of the features mentioned herein results in excellent properties for the present bispecific antibodies such as 100% heterodimer, high yield, high stability and low aggregation propensities.

Accordingly, in an aspect, the present invention provides a bispecific antibody comprising a first binding region that binds to CD3 comprising a first heavy chain variable region (VH1) and a first light chain variable region (VL1), and a second binding region that binds to a tumor associated antigen (TAA) comprising a second heavy chain variable region (VH2) and a second light chain variable region (VL2); wherein the first binding region binds to an epitope of CD3 comprising, within or overlapping with an amino acid sequence of QDGNEEMGGITQTPYKVSISGT (SEQ ID NO: 1); wherein the VH1 and/or the VL1 is linked to a mask peptide via a first linker; wherein the mask peptide comprises the amino acid sequence of SEQ ID NO: 1 or a fragment thereof with at least 5 amino acids in length; and wherein the first linker comprises a protease cleavage site that is specifically cleaved by a protease expressed or overexpressed in tumor microenvironment.

In some embodiments of the bispecific antibody disclosed herein, the epitope of CD3 comprises the amino acid sequence of SEQ ID NO: 1. In some embodiments, the epitope of CD3 is located within the amino acid sequence of SEQ ID NO: 1. In some embodiments, the epitope of CD3 is overlapped with the amino acid sequence of SEQ ID NO: 1.

In some embodiments, the mask peptide comprises a fragment of at least 5 amino acids, preferably at least 7 amino acids, such as 7-15 amino acids, at N terminal of SEQ ID NO: 1, or any combination thereof. In some embodiments, the mask peptide comprises 5 amino acids at N terminal of SEQ ID NO: 1. In some embodiments, the mask peptide comprises 6 amino acids at N terminal of SEQ ID NO: 1. In some embodiments, the mask peptide comprises 7 amino acids at N terminal of SEQ ID NO: 1. In some embodiments, the mask peptide comprises 8 amino acids at N terminal of SEQ ID NO: 1. In some embodiments, the mask peptide comprises 9 amino acids at N terminal of SEQ ID NO: 1. In some embodiments, the mask peptide comprises 10 amino acids at N terminal of SEQ ID NO: 1. In some embodiments, the mask peptide comprises 11 amino acids at N terminal of SEQ ID NO: 1. In some embodiments, the mask peptide comprises 12 amino acids at N terminal of SEQ ID NO: 1. In some embodiments, the mask peptide comprises 13 amino acids at N terminal of SEQ ID NO: 1. In some embodiments, the mask peptide comprises 14 amino acids at N terminal of SEQ ID NO: 1. In some embodiments, the mask peptide comprises 15 amino acids at N terminal of SEQ ID NO: 1. In some embodiments, the mask peptide comprises 16 amino acids at N terminal of SEQ ID NO: 1. In some embodiments, the mask peptide comprises 17 amino acids at N terminal of SEQ ID NO: 1. In some embodiments, the mask peptide comprises 18 amino acids at N terminal of SEQ ID NO: 1. In some embodiments, the mask peptide comprises 19 amino acids at N terminal of SEQ ID NO: 1. In some embodiments, the mask peptide comprises 20 amino acids at N terminal of SEQ ID NO: 1. In some embodiments, the mask peptide comprises 21 amino acids at N terminal of SEQ ID NO: 1.

In some embodiments, the mask peptide comprises an amino acid sequence as set forth in any of SEQ ID NOs: 1-4, or any combination thereof.

In some embodiments, the mask peptide comprises:
M1-L-M2,
wherein M1 and M2 are each independently comprises an amino acid sequence as set forth in any of SEQ ID NOs: 1-4, and L is an optional linker, preferably the linker is (GGGS)n or (GGGGS)n, wherein n is an integer selected from 1-4. In a preferred embodiment, the linker is GGGS (SEQ ID NO: 88).

In some embodiments, M1 comprises an amino acid sequence as set forth in SEQ ID NO: 1, M2 comprises an amino acid sequence as set forth in any of SEQ ID NOs: 1-4, and L is an optional linker, preferably the linker is (GGGS)n or (GGGGS)n, wherein n is an integer selected from 1-4. In a preferred embodiment, the linker is GGGS (SEQ ID NO: 88).

In some embodiments, M1 comprises an amino acid sequence as set forth in SEQ ID NO: 2, M2 comprises an amino acid sequence as set forth in any of SEQ ID NOs: 1-4, and L is an optional linker, preferably the linker is (GGGS)n or (GGGGS)n, wherein n is an integer selected from 1-4. In a preferred embodiment, the linker is GGGS (SEQ ID NO: 88).

In some embodiments, M1 comprises an amino acid sequence as set forth in SEQ ID NO: 3, M2 comprises an amino acid sequence as set forth in any of SEQ ID NOs: 1-4, and Lis an optional linker, preferably the linker is (GGGS)n or (GGGGS)n, wherein n is an integer selected from 1-4. In a preferred embodiment, the linker is GGGS (SEQ ID NO: 88).

In some embodiments, M1 comprises an amino acid sequence as set forth in SEQ ID NO: 4, M2 comprises an amino acid sequence as set forth in any of SEQ ID NOs: 1-4, and L is an optional linker, preferably the linker is (GGGS)n or (GGGGS)n, wherein n is an integer selected from 1-4. In a preferred embodiment, the linker is GGGS (SEQ ID NO: 88).

In some embodiments, the mask peptide comprises an amino acid sequence selected from SEQ ID NOs: 5-8.

In some embodiments, the mask peptide comprises:

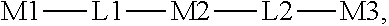

wherein M1, M2 and M3 are each independently comprises an amino acid sequence as set forth in any of SEQ ID NOs: 1-4, and each of L1 and L2 is an optional linker, preferably L1 and L2 are each independently (GGGS)n or (GGGGS)n, wherein n is an integer selected from 1-4. In a preferred embodiment, the linker is GGGS (SEQ ID NO: 88).

In some embodiments, M1 comprises an amino acid sequence as set forth in SEQ ID NO: 1, M2 comprises an amino acid sequence as set forth in any of SEQ ID NOs: 1-4, M3 comprises an amino acid sequence as set forth in any of SEQ ID NOs: 1-4, and each of L1 and L2 is an optional linker, preferably L1 and L2 are each independently (GGGS)n or (GGGGS)n, wherein n is an integer selected from 1-4. In a preferred embodiment, the linker is GGGS (SEQ ID NO: 88).

In some embodiments, M1 comprises an amino acid sequence as set forth in SEQ ID NO: 2, M2 comprises an amino acid sequence as set forth in any of SEQ ID NOs: 1-4, M3 comprises an amino acid sequence as set forth in any of SEQ ID NOs: 1-4, and each of L1 and L2 is an optional linker, preferably L1 and L2 are each independently (GGGS)n or (GGGGS)n, wherein n is an integer selected from 1-4. In a preferred embodiment, the linker is GGGS (SEQ ID NO: 88).

In some embodiments, M1 comprises an amino acid sequence as set forth in SEQ ID NO: 3, M2 comprises an amino acid sequence as set forth in any of SEQ ID NOs: 1-4, M3 comprises an amino acid sequence as set forth in any of SEQ ID NOs: 1-4, and each of L1 and L2 is an optional linker, preferably L1 and L2 are each independently (GGGS)n or (GGGGS)n, wherein n is an integer selected from 1-4. In a preferred embodiment, the linker is GGGS (SEQ ID NO: 88).

In some embodiments, M1 comprises an amino acid sequence as set forth in SEQ ID NO: 4, M2 comprises an amino acid sequence as set forth in any of SEQ ID NOs: 1-4, M3 comprises an amino acid sequence as set forth in any of SEQ ID NOs: 1-4, and each of L1 and L2 is an optional linker, preferably L1 and L2 are each independently (GGGS)n or (GGGGS)n, wherein n is an integer selected from 1-4. In a preferred embodiment, the linker is GGGS (SEQ ID NO: 88).

In some embodiments, the VH1 comprises HCDRs 1-3 having the amino acid sequences as set forth in SEQ ID NOs: 9-11 respectively, and the VL1 comprises LCDRs 1-3 having the amino acid sequences as set forth in SEQ ID NOs: 12-14 respectively.

In some embodiments, CDR sequences are defined according to Kabat numbering system.

When CDR sequences are defined according to Kabat numbering system, the VH1 comprises HCDR1, HCDR2 and HCDR3 having the amino acid sequences as set forth in SEQ ID NO: 9 (GFTFNTYA), SEQ ID NO: 10 (IRSKYNNYAT) and SEQ ID NO: 11 (ARHGNFGSSYVSYFAY) respectively, and the VL1 comprises LCDR1, LCDR2 and LCDR3 having the amino acid sequences as set forth in SEQ ID NO: 12 (TGAVTTSNY), SEQ ID NO: 13 (GAN) and SEQ ID NO: 14 (ALWYSNLWV) respectively.

In some embodiments, the VH1 comprises an amino acid sequence as set forth in SEQ ID NO: 15 or an amino acid sequence having at least 80%, at least 85%, at least 90%, at least 95%, at least 98%, or at least 99% sequence identity to SEQ ID NO: 15, and the VL1 comprises an amino acid sequence as set forth in SEQ ID NO: 16 or an amino acid sequence having at least 80%, at least 85%, at least 90%, at least 95%, at least 98%, or at least 99% sequence identity to SEQ ID NO: 16.

In some embodiments, the VH1 comprises a functional variant of the amino acid sequence as set forth in SEQ ID NO: 15 formed by insertion, deletion and/or substitution of one or more amino acid(s) therein, provided that the functional variant retains the ability of binding to CD3, and the VL1 comprises a functional variant of the amino acid sequence as set forth in SEQ ID NO: 16 formed by insertion, deletion and/or substitution of one or more amino acid(s) therein, provided that the functional variant retains the ability of binding to CD3.

The functional variant comprises or consists of an amino acid sequence having at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to the amino acid sequence of the parent polypeptide.

In the context of the functional variant, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-20, preferably 1-10, more preferably 1-7, still more preferably 1-5, and most preferably 1-2. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3, and/or FR4.

In some embodiments, the substitution of one or more amino acid(s) can be conservative substitution of one or more amino acid(s). Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, He, Val and Cys; and (c) aromatic residues: Phe, Tyr and Trp.

Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In a preferred embodiment, the VH1 comprises an amino acid sequence as set forth in SEQ ID NO: 15, and the VL1 comprises an amino acid sequence as set forth in SEQ ID NO: 16.

In some embodiments, the protease is any protease expressed or overexpressed in tumor microenvironment. For example, the protease can be selected from the group consisting of urokinase (uPA), MMP (e.g., MMP2, MMP9, MMP10, MMP11, MMP12, MMP13, MMP14, MMP15), matriptase B, cathepsin B, cathepsin D, collagenase, stromelysin, TGFβ, plasminogen, staphylokinase, factor Xa, human liver collagen, human α2M, and human PZP. In a preferred embodiment, the protease is selected from the group consisting of uPA, MMP (e.g., MMP2, MMP9, MMP10, MMP11, MMP12, MMP13, MMP14, MMP15), and matriptase B. In a more preferred embodiment, the protease is selected from the group consisting of uPA, MMP9, and matriptase B.

In some embodiments, the protease cleavage site comprises an amino acid sequence selected from any of SEQ ID NOs: 17-20. In some embodiments, the protease cleavage site comprises an amino acid sequence selected from RRXaa, LXaa, AXaa, FXaa or WXaa, wherein Xaa is any amino acid. In some embodiments, the protease cleavage site comprises an amino acid sequence selected from FXaa, YXaa or LXaa, wherein Xaa is any amino acid, preferably is not Ala or Val.

In some embodiments, the protease is uPA and the protease cleavage site comprises the amino acid sequence of SEQ ID NO: 17 (LSGRSDNH). In some embodiments, the protease is MMP2 and the protease cleavage site comprises the amino acid sequence of SEQ ID NO: 18 (PLGLAG). In some embodiments, the protease is matriptase B and the protease cleavage site comprises the amino acid sequence of SEQ ID NO: 19 (PMAKK). In some embodiments, the protease is MMP9-15 and the protease cleavage site comprises the amino acid sequence of SEQ ID NO: 20 (PGPAGMKGLP). In some embodiments, the protease is MMP9 and the protease cleavage site comprises the amino acid sequence of SEQ ID NO: 20 (PGPAGMKGLP). In some embodiments, the protease is Cathepsin B and the protease cleavage site comprises the amino acid sequence, RRXaa, LXaa, AXaa, FXaa, or WXaa (Xaa is any amino acid). In some embodiments, the protease is Cathepsin D and the protease cleavage site comprises the amino acid sequence, FXaa, YXaa or LXaa (Xaa is any amino acid, preferably is not Ala or Val).

In some embodiments, the first linker comprises two or more protease cleavage sites connected in tandem. In some embodiments, the first linker comprises two or more selected from uPA protease cleavage site, MMP protease cleavage site, matriptase B protease cleavage site, cathepsin B protease cleavage site, cathepsin D protease cleavage site, collagenase protease cleavage site, stromelysin protease cleavage site, connected in tandem. In some embodiments, the first linker comprises two or more protease cleavage sites selected from SEQ ID NOs: 17-20 connected in tandem.

In some embodiments, the first linker comprises an amino acid sequence selected from SEQ ID NOs: 21-30.

In some embodiments, the bispecific antibody disclosed herein comprises:
(i) a first polypeptide chain comprising, from N terminal to C terminal, the VH1, an heavy chain constant region 1 (CH1), an heavy chain constant region 2 (CH2), and an heavy chain constant region 3 (CH3); and
(ii) a second polypeptide chain comprising, from N terminal to C terminal, the VL1, a light chain constant region (CL), an heavy chain constant region 2 (CH2), and an heavy chain constant region 3 (CH3);
wherein the VH1 and/or the VL1, at its N terminal, is linked to the mask peptide via the first linker; and
(1) wherein the second binding region comprises a scFv comprising the VH2 and the VL2, wherein the scFv is linked to the C terminal of the first polypeptide chain and/or the second polypeptide chain via a linker, or wherein the scFv is linked to the N terminal of the VH1 via a linker and the mask peptide is linked to the N terminal of the VL1 via the first linker, or wherein the scFv is linked to the N terminal of the VL1 via a linker and the mask peptide is linked to the N terminal of the VH1 via the first linker; or
(2) wherein the VH2 is linked to the C terminal of the first polypeptide chain via a linker and the VL2 is linked to the C terminal of the second polypeptide chain via a linker, or wherein the VL2 is linked to the C terminal of the first polypeptide chain via a linker and the VH2 is linked to the C terminal of the second polypeptide chain via a linker; or
(3) wherein the VH2 is linked to the C terminal of the VH1 via a linker and the VL2 is linked to the C terminal of the VL1 via a linker, or wherein the VH2 is linked to the C terminal of the VL1 via a linker and the VL2 is linked to the C terminal of the VH1 via a linker.

In some embodiments, the first polypeptide chain comprises, from N terminal to C terminal, VH1-linker-VH2-CH1-CH2-CH3; and the second polypeptide chain comprises, from N terminal to C terminal, VL1-linker-VL2-CL-CH2-CH3.

In some embodiments, the first polypeptide chain comprises, from N terminal to C terminal, VH1-CH1-CH2-CH3-linker-VH2; and the second polypeptide chain comprises, from N terminal to C terminal, VL1-CL-CH2-CH3-linker-VL2.

In some embodiments, the first polypeptide chain comprises, from N terminal to C terminal, VH1-CH1-CH2-CH3; and the second polypeptide chain comprises, from N terminal to C terminal, scFv-linker-VL1-CL-CH2-CH3.

In some embodiments, the first polypeptide chain comprises, from N terminal to C terminal, VH1-CH1-CH2-CH3-linker-scFv; and the second polypeptide chain comprises, from N terminal to C terminal, VL1-CL-CH2-CH3-linker-scFv.

In some embodiments, the first polypeptide chain comprises, from N terminal to C terminal, the mask peptide-the first linker-VH1-linker-VH2-CH1-CH2-CH3; and the second polypeptide chain comprises, from N terminal to C terminal, VL1-linker-VL2-CL-CH2-CH3.

In some embodiments, the first polypeptide chain comprises, from N terminal to C terminal, the mask peptide-the first linker-VH1-CH1-CH2-CH3; and the second polypeptide chain comprises, from N terminal to C terminal, scFv-linker-VL1-CL-CH2-CH3.

In some embodiments, the first polypeptide chain comprises, from N terminal to C terminal, the mask peptide-the first linker-VH1-linker-VH2-CH1-CH2-CH3; and the second polypeptide chain comprises, from N terminal to C terminal, the mask peptide-the first linker-VL1-linker-VL2-CL-CH2-CH3.

In some embodiments, the first polypeptide chain comprises, from N terminal to C terminal, the mask peptide-the first linker-VH1-CH1-CH2-CH3-linker-VH2; and the second polypeptide chain comprises, from N terminal to C terminal, VL1-CL-CH2-CH3-linker-VL2.

In some embodiments, the first polypeptide chain comprises, from N terminal to C terminal, the mask peptide-the first linker-VH1-CH1-CH2-CH3-linker-scFv; and the second polypeptide chain comprises, from N terminal to C terminal, VL1-CL-CH2-CH3-linker-scFv.

In some embodiments, each of the linkers is independently comprise an amino acid sequence selected from GGGSS(GGGGS)n, (GGGS)n and (GGGGS)n, wherein n is an integer selected from 1-4. In preferred embodiments, each of the linkers is independently comprise an amino acid sequence selected from any of SEQ ID NOs: 47-49.

In some embodiments, the CH1 and the CL are connected to the CH2 of the first and second polypeptide chains via a linker that comprises a shortened hinge sequence as compared to a hinge sequence in normal human IgG.

Many tumor associated antigens (TAAs) associated with specific cancers have been identified in the art. In some embodiments, tumor-associated antigens are antigens that can potentially stimulate an obvious tumor-specific immune response. Some of these antigens are encoded by normal cells, but not necessarily expressed by normal cells. These antigens can be characterized as those that are usually silent (i.e., not expressed) in normal cells, those that are expressed only during certain stages of differentiation, and those that are expressed over time, such as embryonic and fetal antigens. Other cancer antigens are encoded by mutant cell genes such as oncogenes (e.g. activated ras oncogene), suppressor genes (e.g. mutant p53), and fusion proteins produced by internal deletions or chromosomal translocations. Other cancer antigens can be encoded by viral genes, such as those carried on RNA and DNA tumor viruses. Many other tumor associated antigens and antibodies against them are known and/or commercially available, and can also be produced by those skilled in the art.

Examples of tumor associated antigens include but are not limited to 5T4, alphafetoprotein, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFR, EGFRvIII, epithelial tumor antigen, ERBB2, FLT3, folate binding protein, GD2, GD3, HIV-1 envelope glycoprotein gp41, HIV-1 envelope glycoprotein gpl20, melanoma-associated antigen, MUC-1, mutated p53, mutated ras, ROR1, GPC3, VEGFR2, and combinations thereof.

In some embodiments, the TAA is selected from the group consisting of EGFR, EpCAM, HER2, PSMA, gpA33, CD276, CEA, CD19, CD20, CD22, CD30, CD33, CD123, FLT3 and BCMA.

In some embodiments, the TAA is EGFR, and the VH2 comprises HCDRs 1-3 having the amino acid sequences as set forth in SEQ ID NOs: 31-33 respectively, and the VL2 comprises LCDRs 1-3 having the amino acid sequences as set forth in SEQ ID NOs: 34-36 respectively.

In some embodiments, the TAA is EGFR, and the VH2 comprises HCDRs 1-3 having the amino acid sequences as set forth in SEQ ID NOs: 37-39 respectively, and the VL2 comprises LCDRs 1-3 having the amino acid sequences as set forth in SEQ ID NOs: 40-42 respectively.

In some embodiments, CDR sequences are defined according to Kabat numbering system. When using Kabat defined CDR sequences, the VH2 comprises HCDR1, HCDR2 and HCDR3 having the amino acid sequences as shown in SEQ ID NO: 31 (GFTESSYA), SEQ ID NO: 32 (ISYDGSNK) and SEQ ID NO: 33 (ARGYVARGDFDY) respectively, and the VL2 comprises LCDR1, LCDR2 and LCDR3 having the amino acid sequences as shown in SEQ ID NO: 34 (QSMSRW), SEQ ID NO: 35 (ATS) and SEQ ID NO: 36 (QQLDSFPFSFT) respectively; or the VH2 comprises HCDR1, HCDR2 and HCDR3 having the amino acid sequences as shown in SEQ ID NO: 37 (GGSVSSGDYY), SEQ ID NO: 38 (IYYSGNT) and SEQ ID NO: 39 (VRDRVTGAFDI) respectively, and the VL2 comprises LCDR1, LCDR2 and LCDR3 having the amino acid sequences as shown in SEQ ID NO: 40 (QDISNY), SEQ ID NO: 41 (DAS) and SEQ ID NO: 42 (QHFDHLPLA) respectively

In some embodiments, the TAA is EGFR, and the VH2 comprises an amino acid sequence as set forth in SEQ ID NO: 43 or an amino acid sequence having at least 80%, at least 85%, at least 90%, at least 95%, at least 98%, or at least 99% sequence identity to SEQ ID NO: 43, and the VL2 comprises an amino acid sequence as set forth in SEQ ID NO: 44 or an amino acid sequence having at least 80%, at least 85%, at least 90%, at least 95%, at least 98%, or at least 99% sequence identity to SEQ ID NO: 44.

In some embodiments, the TAA is EGFR, and the VH2 comprises an amino acid sequence as set forth in SEQ ID NO: 45 or an amino acid sequence having at least 80%, at least 85%, at least 90%, at least 95%, at least 98%, or at least 99% sequence identity to SEQ ID NO: 45, and the VL2 comprises an amino acid sequence as set forth in SEQ ID NO: 46 or an amino acid sequence having at least 80%, at least 85%, at least 90%, at least 95%, at least 98%, or at least 99% sequence identity to SEQ ID NO: 46.

In some embodiments, the VH2 comprises a functional variant of the amino acid sequence as set forth in SEQ ID NO: 43 or 45 formed by insertion, deletion and/or substitution of one or more amino acid(s) therein, provided that the functional variant retains the ability of binding to EGFR. In some embodiments, the VL2 comprises a functional variant of the amino acid sequence as set forth in SEQ ID NO: 44 or 46 formed by insertion, deletion and/or substitution of one or more amino acid(s) therein, provided that the functional variant retains the ability of binding to EGFR.

In some embodiments, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-20, preferably 1-10, more preferably 1-7, still more preferably 1-5, and most preferably 1-2. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3, and/or FR4.

In some embodiments, the substitution of one or more amino acid(s) can be conservative substitution of one or more amino acid(s). Examples of conservative substitutions are as described above.

In a preferred embodiment, the VH2 comprises an amino acid sequence as set forth in SEQ ID NO: 43 and the VL2 comprises an amino acid sequence as set forth in SEQ ID NO: 44; or the VH2 comprises an amino acid sequence as set forth in SEQ ID NO: 45 and the VL2 comprises an amino acid sequence as set forth in SEQ ID NO: 46.

In some embodiments, the second binding region comprises a scFv comprising an amino acid sequence as set forth in SEQ ID NO: 75 or SEQ ID NO: 76 or an amino acid sequence having at least 80%, at least 85%, at least 90%, at least 95%, at least 98%, or at least 99% sequence identity to SEQ ID NO: 75 or SEQ ID NO: 76.

In some embodiments, the second binding region comprises a functional variant of the amino acid sequence as set forth in SEQ ID NO: 75 or 76 formed by insertion, deletion and/or substitution of one or more amino acid(s) therein, provided that the functional variant retains the ability of binding to EGFR.

In some embodiments, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-20, preferably 1-10, more preferably 1-7, still more preferably 1-5, and most preferably 1-2. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3, and/or FR4.

In some embodiments, the substitution of one or more amino acid(s) can be conservative substitution of one or more amino acid(s). Examples of conservative substitutions are as described above.

The bispecific antibody disclosed herein comprises an Fc region comprising CH2 and CH3 of an antibody.

The Fc region may be of any isotype, including, but not limited to, IgG1, IgG2, IgG3 and IgG4, and may comprise one or more mutations or modifications. In one embodiment, the Fc region is of IgG1 isotype or derived therefrom, optionally with one or more mutations or modifications. In one embodiment, the Fc region is human IgG1 Fc.

In one embodiment, the Fc region is effector-function-deficient. For example, the Fc region may be of an IgG1 isotype, or a non-IgG1 type, e.g. IgG2, IgG3 or IgG4, which has been mutated such that the ability to mediate effector functions, such as ADCC, has been reduced or even eliminated. Such mutations have e.g. been described in Dall'Acqua W F et al., J Immunol. 177 (2): 1129-1138 (2006) and Hezarch M, J Virol.; 75 (24): 12161-12168 (2001).

In one embodiment, the Fc region comprises a mutation removing the acceptor site for Asn-linked glycosylation or is otherwise manipulated to change the glycosylation properties. For example, in an IgG1 Fc region, an N297Q mutation can be used to remove an Asn-linked glycosylation site. Accordingly, in a specific embodiment, Fc region comprises an IgG1 sequence with an N297Q mutation.

In a further embodiment, the Fc region is glyco-engineered to reduce fucose and thus enhance ADCC, e.g. by addition of compounds to the culture media during antibody production as described in US2009317869 or as described in van Berkel et al. (2010) Biotechnol. Bioeng. 105:350 or by using FUT8 knockout cells, e.g. as described in Yamane-Ohnuki et al. (2004) Biotechnol. Bioeng 87:614. ADCC may alternatively be optimized using the method described by Umaña et al. (1999) Nature Biotech 17:176. In a further embodiment, the Fc region has been engineered to enhance complement activation, e.g. as described in Natsume et al. (2009) Cancer Sci. 100:2411.

In some embodiments, the Fc region comprises modifications or mutations that can inhibit Fc homodimerization. In some embodiments, the Fc region comprises a variant of a human IgG1 Fc wildtype sequence. The variant can comprise amino acid substitutions at positions T366 and Y407 of human IgG1 (Kabat numbering). Preferably, T366 is substituted with L (Leucine). Preferably, Y407 is substituted with I(Isoleucine), F(Phenylalanine), L(Leucine), M(Methionine), H(Histidine), K(Lysine), S(Serine), Q(Glutamine), T(Threonine), W(Tryptophan), A(Alanine), G(Glycine) or N (Asparagine). More preferably, Y407 is substituted with H. In one embodiment, T366 is substituted with L, and Y407 is substituted with H.

In some embodiments, the Fc region can be a monomeric human IgG1 Fc (e.g., mFc7.2) as described in PCT application No. PCT/US2018/016524, which is incorporated herein by reference in its entirety.

In some embodiments, the bispecific antibody comprises a first polypeptide chain comprising an Fc region comprising CH2 and CH3; and a second polypeptide chain comprising an Fc region comprising CH2 and CH3. The Fc region can be those as describe above.

In some embodiments, one or both of the CH3 comprise at least one amino acid mutation that is capable of decreasing homodimerization between the first and second polypeptide chains. In preferred embodiments, amino acid T366 of the one or both CH3 is substituted with L (Leucine), and amino acid Y407 of the one or both CH3 is substituted with I (Isoleucine), F (Phenylalanine), L (Leucine), M (Methionine), H (Histidine), K (Lysine), S (Serine), Q (Glutamine), T (Threonine), W (Tryptophan), A (Alanine), G (Glycine) or N (Asparagine). In one embodiment, amino acid T366 of the one or both CH3 is substituted with L, and amino acid Y407 of the one or both CH3 is substituted with H. In a preferred embodiment, in the CH3 of both the first and second polypeptide chains, T366 is substituted with L and Y407 is substituted with H.

In some embodiments, one or both of the CH2 comprise at least one amino acid mutation that is capable of decreasing the effector function of the bispecific antibody, preferably the at least one mutation is selected from L234A, L235A, G237A, or any combination thereof. In a preferred embodiment, one or both of the CH2 comprise amino acid mutations, L234A, L235A and G237A. In a more preferred embodiment, both of the CH2 in the first and second polypeptide chains comprise amino acid mutations, L234A, L235A and G237A.

In some embodiments, the bispecific antibody disclosed herein comprises:
a first polypeptide chain comprising an amino acid as set forth in SEQ ID NO: 51 or an amino acid sequence having at least 80%, at least 85%, at least 90%, at least 95%, at least 98%, or at least 99% sequence identity to SEQ ID NO: 51, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 52 or an amino acid sequence having at least 80%, at least 85%, at least 90%, at least 95%, at least 98%, or at least 99% sequence identity to SEQ ID NO: 52, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 53 or an amino acid sequence having at least 80%, at least 85%, at least 90%, at least 95%, at least 98%, or at least 99% sequence identity to SEQ ID NO: 53, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 54 or an amino acid sequence having at least 80%, at least 85%, at least 90%, at least 95%, at least 98%, or at least 99% sequence identity to SEQ ID NO: 54, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 55 or an amino acid sequence having at least 80%, at least 85%, at least 90%, at least 95%, at least 98%, or at least 99% sequence identity to SEQ ID NO: 55, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 56 or an amino acid sequence having at least 80%, at least 85%, at least 90%, at least 95%, at least 98%, or at least 99% sequence identity to SEQ ID NO: 56, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 52 or an amino acid sequence having at least 80%, at least 85%, at least 90%, at least 95%, at least 98%, or at least 99% sequence identity to SEQ ID NO: 52, and a second polypeptide chain comprising an amino acid as set forth in SEQ ID NO: 57 or an amino acid sequence having at least 80%, at least 85%, at least 90%, at least 95%, at least 98%, or at least 99% sequence identity to SEQ ID NO: 57; or
a first polypeptide chain comprising an amino acid as set forth in SEQ ID NO: 58 or an amino acid sequence having at least 80%, at least 85%, at least 90%, at least 95%, at least 98%, or at least 99% sequence identity to SEQ ID NO: 58, and a second polypeptide chain comprising an amino acid as set forth in SEQ ID NO: 59 or an amino acid sequence having at least 80%, at least 85%, at least 90%, at least 95%, at least 98%, or at least 99% sequence identity to SEQ ID NO: 59; or
a first polypeptide chain comprising an amino acid as set forth in SEQ ID NO: 60 or an amino acid sequence having at least 80%, at least 85%, at least 90%, at least 95%, at least 98%, or at least 99% sequence identity to SEQ ID NO: 60, and a second polypeptide chain comprising an amino acid as set forth in SEQ ID NO: 61 or an amino acid sequence having at least 80%, at least 85%, at least 90%, at least 95%, at least 98%, or at least 99% sequence identity to SEQ ID NO: 61; or
a first polypeptide chain comprising an amino acid as set forth in SEQ ID NO: 62 or an amino acid sequence having at least 80%, at least 85%, at least 90%, at least 95%, at least 98%, or at least 99% sequence identity to SEQ ID NO: 62, and a second polypeptide chain comprising an amino acid as set forth in SEQ ID NO: 63 or an amino acid sequence having at least 80%, at least 85%, at least 90%, at least 95%, at least 98%, or at least 99% sequence identity to SEQ ID NO: 63; or
a first polypeptide chain comprising an amino acid as set forth in SEQ ID NO: 64 or an amino acid sequence having at least 80%, at least 85%, at least 90%, at least 95%, at least 98%, or at least 99% sequence identity to SEQ ID NO: 64, and a second polypeptide chain comprising an amino acid as set forth in SEQ ID NO: 65 or an amino acid sequence having at least 80%, at least 85%, at least 90%, at least 95%, at least 98%, or at least 99% sequence identity to SEQ ID NO: 65; or
a first polypeptide chain comprising an amino acid as set forth in SEQ ID NO: 66 or an amino acid sequence having at least 80%, at least 85%, at least 90%, at least 95%, at least 98%, or at least 99% sequence identity to SEQ ID NO: 66, and a second polypeptide chain comprising an amino acid as set forth in SEQ ID NO: 67 or an amino acid sequence having at least 80%, at least 85%, at least 90%, at least 95%, at least 98%, or at least 99% sequence identity to SEQ ID NO: 67; or
a first polypeptide chain comprising an amino acid as set forth in SEQ ID NO: 66 or an amino acid sequence having at least 80%, at least 85%, at least 90%, at least 95%, at least 98%, or at least 99% sequence identity to SEQ ID NO: 66, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 69 or an amino acid sequence having at least 80%, at least 85%, at least 90%, at least 95%, at least 98%, or at least 99% sequence identity to SEQ ID NO: 69, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 70 or an amino acid sequence having at least 80%, at least 85%, at least 90%, at least 95%, at least 98%, or at least 99% sequence identity to SEQ ID NO: 70, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 71 or an amino acid sequence having at least 80%, at least 85%, at least 90%, at least 95%, at least 98%, or at least 99% sequence identity to SEQ ID NO: 71, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 72 or an amino acid sequence having at least 80%, at least 85%, at least 90%, at least 95%, at least 98%, or at least 99% sequence identity to SEQ ID NO: 72, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 73 or an amino acid sequence having at least 80%, at least 85%, at least 90%, at least 95%, at least 98%, or at least 99% sequence identity to SEQ ID NO: 73, and a second polypeptide chain comprising an amino acid as set forth in SEQ ID NO: 74 or an amino acid sequence having at least 80%, at least 85%, at least 90%, at least 95%, at least 98%, or at least 99% sequence identity to SEQ ID NO: 74; or
a first polypeptide chain comprising an amino acid as set forth in SEQ ID NO: 77 or an amino acid sequence having at least 80%, at least 85%, at least 90%, at least 95%, at least 98%, or at least 99% sequence identity to SEQ ID NO: 77, and a second polypeptide chain comprising an amino acid as set forth in SEQ ID NO: 78 or an amino acid sequence having at least 80%, at least 85%, at least 90%, at least 95%, at least 98%, or at least 99% sequence identity to SEQ ID NO: 78.

In some embodiments, the first polypeptide chain comprises a functional variant of the amino acid sequence as set forth in any of SEQ ID NOs: 51, 52, 53, 54, 55, 56, 58, 60, 62, 64, 66, 69, 70, 71, 72, 73, and 77 formed by insertion, deletion and/or substitution of one or more amino acid(s) therein, provided that the functional variant retains the ability of binding to CD3 and EGFR. In some embodiments, the second polypeptide chain comprises a functional variant of the amino acid sequence as set forth in any of SEQ ID NOs: 50, 57, 59, 61, 63, 65, 67, 68, 74 and 78 formed by insertion, deletion and/or substitution of one or more amino acid(s) therein, provided that the functional variant retains the ability of binding to CD3 and EGFR.

In some embodiments, the number of the inserted, deleted and/or substituted amino acid is preferably no more than 40% of the total number of amino acids in the parent amino acid sequence, more preferably no more than 35%, more preferably 1-33%, and more preferably 5-30%, more preferably 10-25%, more preferably 15-20%. For example, the number of the inserted, deleted and/or substituted amino acid can be 1-50, preferably 1-20, more preferably 1-10, still more preferably 1-5. In a preferred embodiment, the number of the inserted, deleted and/or substituted amino acid is 1, 2, 3, 4, 5, 6, or 7.

In some embodiments, the insertion, deletion and/or substitution can be performed at framework (FR) regions, e.g., at FR1, FR2, FR3 and/or FR4; and/or constant regions, e.g., CL, CH1, CH2 and/or CH3.

In some embodiments, the substitution of one or more amino acid(s) can be conservative substitution of one or more amino acid(s). Examples of conservative substitutions are as described above.

In a preferred embodiment, the bispecific antibody disclosed herein comprises:
a first polypeptide chain comprising an amino acid as set forth in SEQ ID NO: 51 and a second polypeptide chain comprising an amino acid as set forth in SEQ ID NO: 50 (1.1 proBiTE); or
a first polypeptide chain comprising an amino acid as set forth in SEQ ID NO: 52 and a second polypeptide chain comprising an amino acid as set forth in SEQ ID NO: 50 (1.2 proBiTE); or
a first polypeptide chain comprising an amino acid as set forth in SEQ ID NO: 53 and a second polypeptide chain comprising an amino acid as set forth in SEQ ID NO: 50 (1.3 proBiTE); or
a first polypeptide chain comprising an amino acid as set forth in SEQ ID NO: 54 and a second polypeptide chain comprising an amino acid as set forth in SEQ ID NO: 50 (1.4 proBiTE); or
a first polypeptide chain comprising an amino acid as set forth in SEQ ID NO: 55 and a second polypeptide chain comprising an amino acid as set forth in SEQ ID NO: 50 (1.4.1 proBiTE); or
a first polypeptide chain comprising an amino acid as set forth in SEQ ID NO: 56 and a second polypeptide chain comprising an amino acid as set forth in SEQ ID NO: 50 (1.4.3 proBiTE); or
a first polypeptide chain comprising an amino acid as set forth in SEQ ID NO: 52 and a second polypeptide chain comprising an amino acid as set forth in SEQ ID NO: 57 (1.5 proBiTE); or
a first polypeptide chain comprising an amino acid as set forth in SEQ ID NO: 58 and a second polypeptide chain comprising an amino acid as set forth in SEQ ID NO: 59 (1.6 proBiTE); or
a first polypeptide chain comprising an amino acid as set forth in SEQ ID NO: 60 and a second polypeptide chain comprising an amino acid as set forth in SEQ ID NO: 61 (1.6.1 proBiTE); or
a first polypeptide chain comprising an amino acid as set forth in SEQ ID NO: 62 and a second polypeptide chain comprising an amino acid as set forth in SEQ ID NO: 63 (1.7 proBiTE); or
a first polypeptide chain comprising an amino acid as set forth in SEQ ID NO: 64 and a second polypeptide chain comprising an amino acid as set forth in SEQ ID NO: 65 (1.7.1 proBiTE); or
a first polypeptide chain comprising an amino acid as set forth in SEQ ID NO: 66 and a second polypeptide chain comprising an amino acid as set forth in SEQ ID NO: 67 (1.6.1M proBiTE); or
a first polypeptide chain comprising an amino acid as set forth in SEQ ID NO: 66 and a second polypeptide chain comprising an amino acid as set forth in SEQ ID NO: 68 (1.6.1M-Pa proBiTE); or
a first polypeptide chain comprising an amino acid as set forth in SEQ ID NO: 69 and a second polypeptide chain comprising an amino acid as set forth in SEQ ID NO: 68 (1.6.1M-2-1-Pa proBiTE); or
a first polypeptide chain comprising an amino acid as set forth in SEQ ID NO: 70 and a second polypeptide chain comprising an amino acid as set forth in SEQ ID NO: 68 (1.6.1M-2-2-Pa proBiTE); or
a first polypeptide chain comprising an amino acid as set forth in SEQ ID NO: 71 and a second polypeptide chain comprising an amino acid as set forth in SEQ ID NO: 68 (1.6.1M-2-3-Pa proBiTE); or
a first polypeptide chain comprising an amino acid as set forth in SEQ ID NO: 72 and a second polypeptide chain comprising an amino acid as set forth in SEQ ID NO: 68 (1.6.1M-2-4-Pa proBiTE); or
a first polypeptide chain comprising an amino acid as set forth in SEQ ID NO: 73 and a second polypeptide chain comprising an amino acid as set forth in SEQ ID NO: 74 (1.4.3M-Pa proBiTE); or
a first polypeptide chain comprising an amino acid as set forth in SEQ ID NO: 77 and a second polypeptide chain comprising an amino acid as set forth in SEQ ID NO: 78 (1.4.3-Pa proBiTE).

In another aspect, the present invention provides a nucleic acid comprising a nucleotide sequence encoding the bispecific antibody disclosed herein.

In still another aspect, the present invention provides a vector comprising the nucleic acid disclosed herein.

Any vector may be suitable for the present disclosure. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof. Suitable exemplary vectors include e.g., pBY, pGAR, pBABE-puro, pBABE-neo largeTcDNA, pBABE-hygro-hTERT, pMKO.1 GFP, MSCV-IRES-GFP, pMSCV PIG (Puro IRES GFP empty plasmid), pMSCV-loxp-dsRed-loxp-cGFP-Puro-WPRE, MSCV IRES Luciferase, pMIG, MDH1-PGK-GFP_2.0, TtRMPVIR, pMSCV-IRES-mCherry FP, pRetroX GFP T2A Cre, pRXTN, pLncEXP, and pLXIN-Luc.

A recombinant expression vector may be any suitable recombinant expression vector. Suitable vectors comprise those designed for propagation and expansion or for expression or both, such as plasmids and viruses. For example, a vector may be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also may be used. Examples of plant expression vectors useful in the context of the disclosure comprise pBY, pBI01, pBI101.2, pBI101.3, pBI121 and pBIN19 (Clontech). Examples of animal expression vectors useful in the context of the disclosure comprise pcDNA, pEUK-Cl, pMAM, and pMAMneo (Clontech).

Recombinant expression vectors may be prepared using standard recombinant DNA techniques. Constructs of expression vectors, which are circular or linear, may be prepared to contain a replication system functional in a prokaryotic or eukaryotic host cell. Replication systems may be derived, e.g., from ColEl, 2μ plasmid, λ, SV40, bovine papilloma virus, and the like.

In yet another aspect, the present invention provides a host cell comprising the nucleic acid or the vector disclosed herein.

Any cell may be used as a host cell for the nucleic acids or the vectors of the present disclosure. In some embodiments, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. aeruginosa; and Streptomyces. In some embodiments, the cell is a human cell. In some embodiments, the cell is an immune cell. In some embodiments, host cells include, for example, CHO cells, such as CHOS cells and CHO-K1 cells, or HEK293 cells, such as HEK293A, HEK293T and HEK293FS.

In still another aspect, the present invention provides a pharmaceutical composition comprising (i) the bispecific antibody disclosed herein; and (ii) a pharmaceutically acceptable carrier or excipient.

In some embodiments, the carrier or excipient for use with the composition disclosed herein includes but is not limited to maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

In some embodiments of the pharmaceutical composition disclosed herein, the pharmaceutical composition further comprises a second therapeutic agent.

In some embodiments, the second therapeutic agent is selected from an antibody, a chemotherapeutic agent and a small molecule drug.

In some embodiments, the second therapeutic agent is selected from a Bruton's tyrosine kinase (BTK) inhibitor, a PI3K inhibitor, a HDAC inhibitor, an ERK inhibitor, a MAPK inhibitor, a PD-1/PD-L1 inhibitor, a LAG3 inhibitor, a CTLA-4 inhibitor, a TIGIT inhibitor, a TIM3 inhibitor, or glucocorticoid.

In some embodiments, the therapeutic agent is a chemotherapeutic agent. The chemotherapeutic agents can include, for example, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In yet another aspect, the present invention provides a conjugate comprising the bispecific antibody disclosed herein and a chemical moiety conjugated thereto.

In some embodiments of the conjugate disclosed herein, the chemical moiety is selected from the group consisting of a therapeutic agent, a detectable moiety, and an immune stimulatory molecule.

In some embodiments, the therapeutic agent includes but is not limited to immunomodulators, radioactive compounds, enzymes (for example perforin), chemotherapeutic agents (for example cis-platin), or a toxin. In some embodiments, the therapeutic agent can be such as maytansine, geldanamycin, tubulin inhibitors such as tubulin binding agents (e.g., auristatins), or minor groove binding agents such as calicheamicin.

Other suitable therapeutic agents include such as, small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E vincristine and doxorubicin; peptide cytotoxins, i.e. proteins or fragments thereof with the ability to kill mammalian cells, for example, ricin, diphtheria toxin, pseudomonas bacterial exotoxin A, DNase and RNase; radio-nuclides, i.e. unstable isotopes of elements which decay with the concurrent emission of one or more of a or β particles, or γ rays, for example, iodine-131, rhenium-186, indium-111, yttrium-90, bismuth-210, bismuth-213, actinium-225 and astatine-213; chelating agents may be used to facilitate the association of these radionuclides to the molecules, or multimers thereof.

In some embodiments, the detectable moiety can be selected from the group consisting of biotin, streptavidin, an enzyme or catalytically active fragment thereof, a radionuclide, a nanoparticle, a paramagnetic metal ion, or a fluorescent, phosphorescent, or chemiluminescent molecule. A detectable moiety for diagnostic purposes include for instance, fluorescent labels, radiolabels, enzymes, nucleic acid probes and contrast reagents.

In some embodiments, the immune stimulatory molecule is an immune effector molecules which stimulate immune response. For example, the immune stimulatory molecule can be cytokines such as IL-2 and IFN-γ, chemokines such as IL-8, platelet factor 4, melanoma growth stimulatory protein, complement activators; viral/bacterial protein domains, or viral/bacterial peptides.

In still another aspect, the present invention provides a method of treating a cancer in a subject, comprising administering to the subject an effective amount of the bispecific antibody disclosed herein, the pharmaceutical composition disclosed herein, or the conjugate disclosed herein.

The cancer can be any cancers, such as cancers of blood system, cancers of central and peripheral nervous system, cancers of lymphoid lineage, cancers of bone marrow lineage, cancers derived from mesenchymal, solid tumor, etc.

In some embodiments of the method disclosed herein, the TAA is EGFR and the cancer is an EGFR positive cancer.

In some embodiments, the cancer is selected from the group consisting of fibrosarcoma, colon cancer, colorectal cancer, liver cancer, kidney cancer, lung cancer, gastric cancer, ovarian cancer, breast cancer, pancreatic cancer, head and neck cancer, glioblastoma, and prostatic cancer.

In some embodiments, dosage administered to a subject may vary with the embodiment, the medicament employed, the method of administration, and the site and subject being treated. However, a dose should be sufficient to provide a therapeutic response. A clinician may determine the effective amount to be administered to a human or other subject in order to treat a medical condition. The precise amount required to be therapeutically effective may depend upon numerous factors, e.g., such as the activity of the antibody, and the route of administration.

A dose of the bispecific antibodies, compositions or conjugates described herein may be administered to a mammal at one time or in a series of sub-doses administered over a suitable period of time, e.g., on a daily, semi-weekly, weekly, bi-weekly, semi-monthly, bi-monthly, semi-annual, or annual basis, as needed. A dosage unit comprising an effective amount of bispecific antibodies, compositions or conjugates may be administered in a single daily dose, or the total daily dosage may be administered in two, three, four, or more divided doses administered daily, as needed.

A suitable means of administration may be selected by a medical practitioner. Route of administration may be parenteral, for example, administration by injection, transnasal administration, transpulmonary administration, or transcutaneous administration. Administration may be systemic or local by intravenous injection, intramuscular injection, intraperitoneal injection, subcutaneous injection. In some embodiments, the bispecific antibodies, compositions or conjugates are selected for parenteral delivery, for inhalation, or for delivery through the digestive tract, such as orally. Dose and method of administration may vary depending on the weight, age, condition, and the like of the subject, and may be suitably selected.

In some embodiments, the method further comprises administering to the subject a second therapeutic agent.

In certain embodiments, the bispecific antibody, composition or conjugate disclosed herein is administered prior to, substantially simultaneously with, or after the administration of the second therapeutic agent.

In some embodiments, the second therapeutic agent is selected from an antibody, a chemotherapeutic agent and a small molecule drug.

In some embodiments, the second therapeutic agent is selected from a Bruton's tyrosine kinase (BTK) inhibitor, a PI3K inhibitor, a HDAC inhibitor, an ERK inhibitor, a MAPK inhibitor, a PD-1/PD-L1 inhibitor, a LAG3 inhibitor, a CTLA-4 inhibitor, a TIGIT inhibitor, a TIM3 inhibitor, and glucocorticoid.

In some embodiments, the second therapeutic agent is a chemotherapeutic agent. The chemotherapeutic agents can include, for example, cytotoxic agents, anti-metabolite agents (e.g., folate antagonists, purine analogs, pyrimidine analogs, etc.), topoisomerase inhibitors (e.g., camptothecin derivatives, anthracenedione, anthracyclines, epipodophyllotoxins, quinoline alkaloids, etc.), anti-microtubule agents (e.g., taxanes, vinca alkaloids), protein synthesis inhibitors (e.g., cephalotaxine, camptothecin derivatives, quinoline alkaloids), alkylating agents (e.g., alkyl sulfonates, ethylenimines, nitrogen mustards, nitrosoureas, platinum derivatives, triazenes, etc.), alkaloids, terpenoids, and kinase inhibitors.

In yet another aspect, the present invention provides a pharmaceutical pack or kit comprising one or more containers filled with one or more of the ingredients of the pharmaceutical compositions described herein, such as the bispecific antibodies disclosed herein. Optionally, associated with such container(s) can be a notice in the form prescribed by a governmental agency regulating the manufacture, use or sale of pharmaceuticals or biological products, which notice reflects approval by the agency of manufacture, use or sale for human administration.

In a specific embodiment, the kit comprises a first container containing the bispecific antibodies disclosed herein. In a specific embodiment, the kit comprises a first container that is a vial containing the bispecific antibodies as a lyophilized sterile powder under vacuum, and the kit further comprises a second container comprising a pharmaceutically acceptable fluid.

In a specific embodiment, provided herein is an injection device containing the bispecific antibodies. In a specific embodiment, the injection device comprises the bispecific antibody in sterile solution. In a specific embodiment, the injection device is a syringe.

In another aspect, the present invention provides use of the bispecific antibody disclosed herein, the pharmaceutical composition disclosed herein, or the conjugate disclosed herein in the manufacture of a medicament for treating a cancer in a subject. In some embodiments, the bispecific antibody disclosed herein, the pharmaceutical composition disclosed herein, or the conjugate disclosed herein is in combination with a second therapeutic agent.

In still another aspect, the present invention provides the bispecific antibody disclosed herein, the pharmaceutical composition disclosed herein, or the conjugate disclosed herein for use in treating a cancer in a subject.

In some embodiments of the use disclosed herein, the TAA is EGFR and the cancer is an EGFR positive cancer. In some embodiments, the cancer is selected from the group consisting of fibrosarcoma, colon cancer, colorectal cancer, liver cancer, kidney cancer, lung cancer, gastric cancer, ovarian cancer, breast cancer, pancreatic cancer, head and neck cancer, glioblastoma, and prostatic cancer.

In some embodiments, the second therapeutic agent is selected from an antibody, a chemotherapeutic agent and a small molecule drug. In some embodiments, the second therapeutic agent is selected from a Bruton's tyrosine kinase (BTK) inhibitor, a PI3K inhibitor, a HDAC inhibitor, an ERK inhibitor, a MAPK inhibitor, a PD-1/PD-L1 inhibitor, a LAG3 inhibitor, a CTLA-4 inhibitor, a TIGIT inhibitor, a TIM3 inhibitor, and glucocorticoid.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

293 free style (293FS) cells, CHO-S cells and protein A agarose were purchased from ThermoFisher Scientific. HCT116, HT29 colon cell lines and HuH7 hepatoma cell lines were purchased from ATCC. Other cell lines were purchased from National Collection of Authenticated Cell Cultures.

Recombinant human EGFR, Fc gamma receptor (FcyR), and urokinase (uPA) were purchased from Sino Biological. Human CD3 protein was purchased from ACRO. The pBY vector used for mammalian expression was synthesized and assembled by overlapping PCR and ligation. Human Fab phage libraries used for panning were generated in house. Anti-human IgG (Fc specific)-FITC antibody produced in goat, anti-human IgG-R-PE antibody produced in goat and anti-human IgG (Fc-specific)-peroxidase antibody produced in goat were purchased from Sigma.

### Example 1. Construction and Initial Characterization of CD3×EGFR Bispecific Antibodies

Bispecific T cell engager (BiTE) is a novel class of bispecific antibodies that can guide cytotoxic T cells to kill cancer cells by simultaneously binding to a tumor antigen and the CD3 molecule on T cell surface.

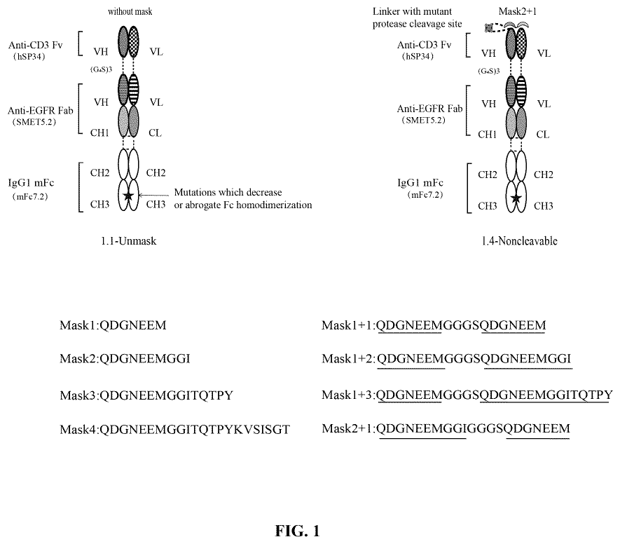

As a negative control, a 1.1-unmask H-shaped BiTE (HBITE) containing neither a protease cleavable site nor a mask peptide was generated. HBiTE is a format consisting of a light chain and a heavy chain forming a heterodimer. The light chain, from N-terminus to C-terminus, comprises an anti-CD3 VL domain, an anti-target VL-CL and a monomeric human IgG1 Fc (e.g., mFc7.2). Monomeric Fc7.2 contains two amino acid mutations (T366L and Y407H) capable of inhibiting Fc homodimerization. The heavy chain, from N-terminus to C-terminus, comprises an anti-CD3 VH domain, an anti-target VH-CH1 and mFc7.2. To generate 1.1-Unmask HBITE (FIG. 1), the VL and VH domains of anti-CD3 antibodies were fused to the N-terminus of the VL and VH domains of an anti-EGFR Fab via a (G4S) 3 linker, respectively. The anti-EGFR Fab was further fused to the N terminus of mFc7.2. The light chain and heavy chain were constructed into a single vector pcDNA3.4 for mammalian cell expression.

For positive control, a 1.4-Noncleavable i-shaped BITE (iBITE) containing only a mask peptide but not a protease cleavable site was designed. In 1.4-Noncleavable iBiTE, the mask peptide gene fragments were fused via a noncleavable linker to the N terminus of the Fv of anti-CD3 antibody (FIG. 1). In this format, binding to CD3 is sterically restricted by the linker. Both light chain and heavy chain were constructed into the vector pcDNA3.4.

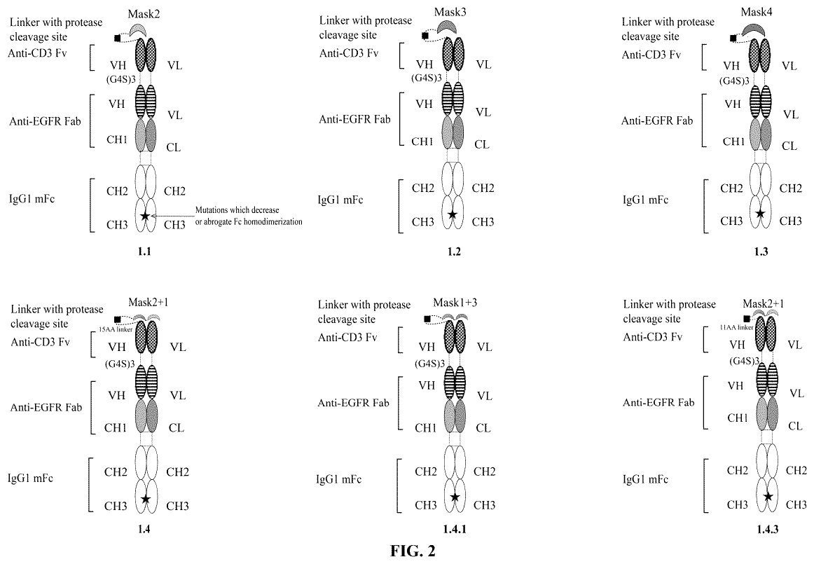

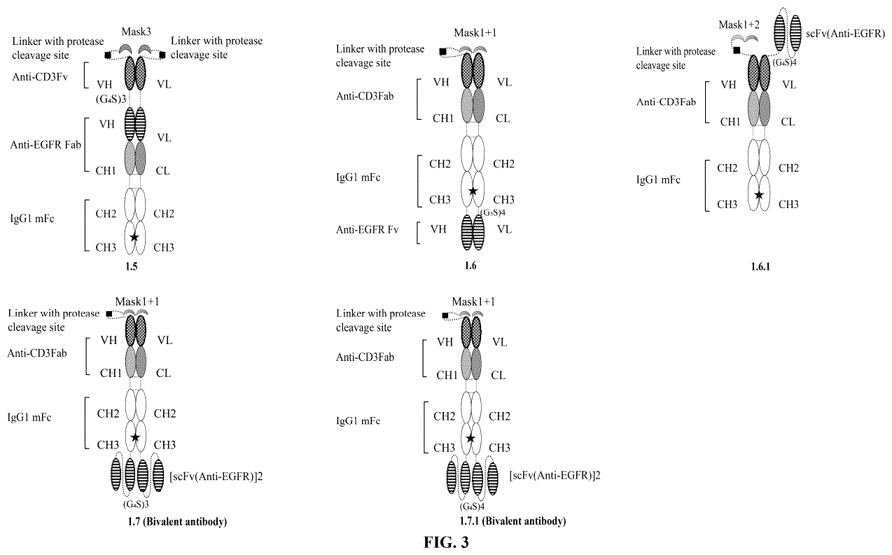

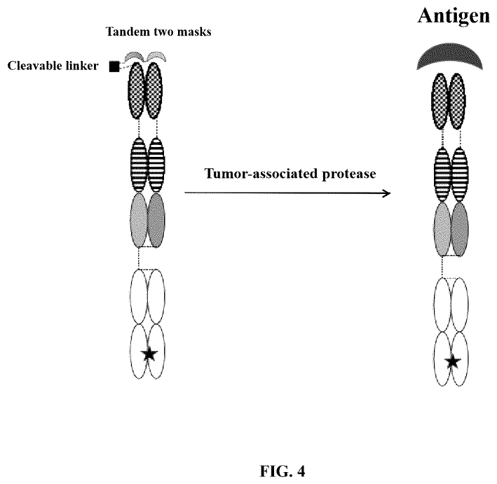

1.1, 1.2, 1.3, 1.4, 1.4.1, 1.4.3, 1.5, 1.6, 1.6.1, 1.7 and 1.7.1 protease cleavable BiTEs (proBiTEs) were efficiently cleaved by a protease expressed or overexpressed in tumor microenvironment (e.g., uPA), resulting in an exposed VH domain of the anti-CD3 antibody (FIGS. 2-3), while 1.1-Unmask HBiTE and 1.4-Noncleavable iBiTE were not sensitive to the protease (FIG. 4).

A mechanism of action of the proBiTEs is proposed as follows: in the tumor microenvironment where protease expression is up-regulated, efficient cleavage of the polypeptide linker by the proteases removes the steric hindrance allowing proBiTEs to bind to the CD3 antigen and exert its functions (FIG. 5). In some design of proBiTEs, mask gene fragments are fused to the N terminus of the VH domain of the Fab of a CD3 antibody via a protease non-cleavable and cleavable polypeptide linker, respectively. In tumor tissues, however, proBiTEs bound to tumor antigens could be stabilized due to cooperation between the VH-VL and antibody-antigen interface, leading to retained full function of the bispecific antibodies even after cleavage by proteases (FIG. 5, right panel). It is therefore hypothesized that this unique proBiTE design could further decrease on-target toxicity of this class of bispecific antibodies. In summary, as compared to published monovalent bispecific antibodies developed by others, the bispecific antibodies of the present invention have the distinctive features of using (i) monomeric Fc domain, for example, human IgG1 Fc, to extend in vivo half-life, and (ii) only two polypeptide chains and employ both Fab and Fv or scFv for heterodimerization and antigen recognition. A combination of the features mentioned herein results in excellent properties for the present bispecific antibodies such as 100% heterodimer, high yield, high stability and low aggregation propensities.

1.1. Cloning of 1.1-Unmask HBiTE, 1.4-Noncleavable iBiTE and 1.1/1.2 proBiTEs

1.1-Unmask HBiTE, 1.1 proBiTE and 1.2 proBiTE were synthesized by Biointron (Shanghai). The gene fragments of VL and VH domains of anti-EGFR Fab were amplified from SMET5.2. The gene fragments of VL and VH domains of anti-CD3 Fv were amplified from hSP34. 1.1 and 1.2 proBiTEs were connected to Mask2 and Mask3 peptides by (Gly₄Ser)₃ linker at the 5′ end of the anti-CD3 Fv region of the heavy chain, respectively (FIG. 2).

1.4-Noncleavable iBiTE shares the same light chain with 1.1-Unmask HBiTE, 1.1 proBiTE and 1.2 proBiTE. The heavy chain of 1.4-Noncleavable iBiTE was constructed by inverse PCR with the following primers:

E005-1.4-NON-HC-FP-M1:
5′-GGAGCAGGGAACCACGGCGGCAGCGAG-3′;
E005-1.4-NON-HC-RP-M:
5′-TCCGCTCAGGCCGCTTCCTCCGCCTGATC-3′.

The full-length amino acid sequences of 1.1-Unmask HBiTE, 1.4-Noncleavable iBiTE and 1.1 and 1.2 proBiTEs are shown as follows.

### 1.1-Unmask HBiTE

Heavy chain
(SEQ ID NO: 79)
EVQLVESGGGLVQPGGSLRLSCAASGFTENTYAMNWVRQAPGKGLEWVARIRSKYNNYATYYADSVKGRFTISRD
DSKNTLYLQMNSLRAEDTAVYYCARHGNFGSSYVSYFAYWGQGTTVTVSSGGGSSGGGGSGGGGSEVQLVETG
GGVVQPGRSLRLSCAASGFTESSYAMHWVRQAPGKGLEWVAVISYDGSNKYYADSVKGRFTISRDNS
KNTLYLQMNSLRAEDTAVYYCARGYVARGDEDYWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPKS
CPPCPAPELLGGPSVFLFPPKPKDTLMISRTPEVTCVVVDVSHEDPEVKENWYVDGVEVHNAKTKPREEQYNSTY
RVVSVLTVLHQDWLNGKEYKCKVSNKALPAPIEKTISKAKGQPREPQVYTLPPSRDELTKNQVSLLCLVKGFYPSD
LAVEWESNGQPENNYKTTPPVLDSDGSFFLHSKLTVDKSRWQQGNVESCSVMHEALHNHYTQKSLSLSPGK,

wherein anti-CD3 VH is shown as italics and HCDRs 1-3 therein is double underlined; anti-EGFR VH is shown as bold and HCDRs 1-3 therein is double underlined; and CH1-CH2-CH3 is shown as italics and double underline.

Light chain
(SEQ ID NO: 50)
EIVVTQSPATLSVSPGERATLSCRSSTGAVTTSNYANWVQQKPGQAPRGLIGGANKRAPGVPARFSGSLSGDEATLT
ISSLQSEDFAVYYCALWYSNLWVFGQGTKLEIKGGGGSGGGGSGGGGSDVVMTQSPSTLSASVGDRVTITCR
ASQSMSRWLAWYQQKPGKGPKLLIYATSTLQSGVPSRFSGSGSGTEFTLTISSLQPEDFATYYCQQLD
SFPFSFTFGPGTKVDLKRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSENRGECPPCPAPELLGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLLCLVKGFYPSDIAVEWESNGQPENNYKTTPPVLDSDGSE
FLHSKLTVDKSRWQQGNVFSCSVMHEALHNHYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline.
1.4-Noncleavable iBiTE

Heavy chain
(SEQ ID NO: 80)

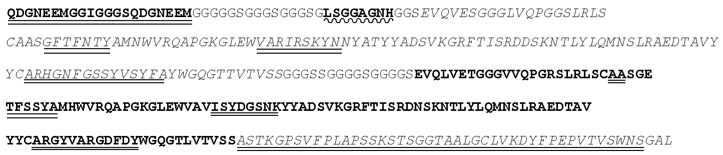

TSGVHTFPAVLOSSGLYSLSSVVTVPSSSLGTQTYICNVNHKPSNTKVDKKVEPKSCPPCPAPELLGGPSVFLFPPK
PKDTLMISRTPEVTCVVVDVSHEDPEVKENWYVDGVEVHNAKTKPREEQYNSTYRVVSVLTVLHQDWLNGKEYK
CKVSNKALPAPIEKTISKAKGOPREPOVYTLPPSRDELTKNQVSLLCLVKGFYPSDIAVEWESNGQPENNYKTTPP
VLDSDGSFFLHSKLTVDKSRWQQGNVFSCSVMHEALHNHYTOKSLSLSPGK,

wherein anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline; the mask peptide is shown as bold and double underline; and the mutant protease cleavage site is shown as bold and wavy line.

Light chain
(SEQ ID NO: 50)
EIVVTQSPATLSVSPGERATLSCRSSTGAVTTSNY
ANWVQQKPGQAPRGLIGGANKRAPGVPARFSGSLS
GDEATLTISSLQSEDFAVYYC
ALWYSNLWVFGQGTKLEIKGGGGSGGG
GSGGGGSDVVMTQSPSTLSASVGDRVTITCRAS
QSMSRWLAWYQQKPGKGPKLLIYATSTLQSGV
PSRFSGSGSGTEFTLTISSLQPEDFATYYC
QQLDSFPFSFTFGPGTKVDLK
RTVAAPSVFIFPPSDEQLKSGTASVVCLLNNFYPREAKVQWK
VDNALQSGNSQESVTEQDSKDSTYSLSSTLTLSKADYEKHKV
YACEVTHQGLSSPVTKSFNRGECPPCPAPELLGGPSVFLFPP
KPKDTLMISRTPEVTCVVVDVSHEDPEVKFNWYVDGVEVHNA
KTKPREEQYNSTYRVVSVLTVLHQDWLNGKEYKCKVSNKALP
APIEKTISKAKGQPREPQVYTLPPSRDELTKNQVSLLCLVKG
FYPSDIAVEWESNGQPENNYKTTPPVLDSDGSFFLHSKLTVD
KSRWQQGNVFSCSVMHEALHNHYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline.
1.1 proBiTE

Heavy chain
(SEQ ID NO: 51)
QDGNEEMGGIGGGSGGGSGGGSG [symbol] GGS
EVQLVESGGGLVQPGGSLRLSCAASGFTFNTYA
MNWVRQAPGKGLEWVARIRSKYNNYATYYADSVKG
RFTISRDDSKNTLYLQMNSLRAEDTAVYYCARHG
NFGSSYVSYFAYWGQGTTVTVSSGGGSSGGGGSGGGGS
EVQLVETGGGVVQPGRSLRLSCAAS
GFTFSSYAMHWVRQAPGKGLEWVAV
ISYDGSNKYYADSVKGRFTISRDN
SKNTLYLQMNSLRAEDTAVYYC
ARGYVARGDFDYWGQGTLVTVSS
ASTKGPSVFPLAPSSKSTSGGTAALGCLVKDYFPE
PVTVSWNSGALTSGVHTFPAVLQSSGLYSLSSVVT
VPSSSLGTQTYICNVNHKPSNTKVDKKVEPKSCPP
CPAPELLGGPSVFLFPPKPKDTLMISRTPEVTCVV
VDVSHEDPEVKFNWYVDGVEVHNAKTKPREEQYNS
TYRVVSVLTVLHQDWLNGKEYKCKVSNKALPAPIE
KTISKAKGQPREPQVYTLPPSRDELTKNQVSLLCL
VKGFYPSDIAVEWESNGQPENNYKTTPPVLDSDGS
FFLHSKLTVDKSRWQQGNVFSCSVMHEALHNHYTQ
KSLSLSPGK,

wherein anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline; the mask peptide is shown as bold and double underline; and the protease cleavage site is shown as bold and wavy line.

Light chain
(SEQ ID NO: 50)
EIVVTQSPATLSVSPGERATLSCRSSTGAVTTSNY
ANWVQQKPGQAPRGLIGGANKRAPGVPARFSGSLS
GDEATLTISSLQSEDFAVYYCALWYSNLWVFGQGTKLEIK
GGGGSGGGGSGGGGSDVVMTQSPSTLSASVGD
RVTITCRASQSMSRWLAWYQQKPGKGPKLLIY
ATSTLQSGVPSRFSGSGSGTEFTLTISSLQPED
FATYYCQQLDSFPFSFTFGPGTKVDLK
RTVAAPSVFIFPPSDEQLKSGTASVVCLLNNFYPREAKVQ
WKVDNALQSGNSQESVTEQDSKDSTYSLSSTLTLSKADYE
KHKVYACEVTHQGLSSPVTKSFNRGECPPCPAPELLGGPS
VFLFPPKPKDTLMISRTPEVTCVVVDVSHEDPEVKFNWYV
DGVEVHNAKTKPREEQYNSTYRVVSVLTVLHQDWLNGKEY
KCKVSNKALPAPIEKTISKAKGQPREPQVYTLPPSRDELT
KNQVSLLCLVKGFYPSDIAVEWESNGQPENNYKTTPPVLD
SDGSFFLHSKLTVDKSRWQQGNVFSCSVMHEALHNHYTQK
SLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline.
1.2 proBiTE

Heavy chain
(SEQ ID NO: 52)
QDGNEEMGGITQTPYGGGSGGGSGGGSG [symbol]
GGSEVQLVESGGGLVQPGGSLRLSCAASGFTFNTYA
MNWVRQAPGKGLEWVARIRSKYNNYATYYADSVKGR
FTISRDDSKNTLYLQMNSLRAEDTAVYYCARHGNFGSS
YVSYFAYWGQGTTVTVSSGGGSSGGGGSGGGGS
EVQLVETGGGVVQPGRSLRLSCAASGFTFSSYAMHW
VRQAPGKGLEWVAVISYDGSNKYYADSVKGRFTISR
DNSKNTLYLQMNSLRAEDTAVYYCARGYVARGDFDY
WGQGTLVTVSSASTKGPSVFPLAPSSKSTSGGTAAL
GCLVKDYFPEPVTVSWNSGALTSGVHTFPAVLQSSG
LYSLSSVVTVPSSSLGTQTYICNVNHKPSNTKVDKK
VEPKSCPPCPAPELLGGPSVFLFPPKPKDTLMISRTP
EVTCVVVDVSHEDPEVKFNWYVDGVEVHNAKTKPREE
QYNSTYRVVSVLTVLHQDWLNGKEYKCKVSNKALPAP
IEKTISKAKGQPREPQVYTLPPSRDELTKNQVSLLCL
VKGFYPSDIAVEWESNGQPENNYKTTPPVLDSDGSFF
LHSKLTVDKSRWQQGNVFSCSVMHEALHNHYTQKSLS
LSPGK,

wherein anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline; the mask peptide is shown as bold and double underline; and the protease cleavage site is shown as bold and wavy line.

Light chain
(SEQ ID NO: 50)
EIVVTQSPATLSVSPGERATLSCRSSTGAVTTSNYANWV
QQKPGQAPRGLIGGANKRAPGVPARFSGSLSGDEATLT
ISSLQSEDFAVYYCALWYSNLWVFGQGTKLEIKGGGGSG
GGGSGGGGSDVVMTQSPSTLSASVGDRVTITCR
ASQSMSRWLAWYQQKPGKGPKLLIYATSTLQSG
VPSRFSGSGSGTEFTLTISSLQPEDFATYYCQQLD
SFPFSFTFGPGTKVDLKRTVAAPSVFIFPPSDEQLKSGT
ASVVCLLNNFYPREAKVQWKVDNALQSGNSQESVTEQDS
KDSTYSLSSTLTLSKADYEKHKVYACEVTHQGLSSPVTK
SFNRGECPPCPAPELLGGPSVFLFPPKPKDTLMISRTPE
VTCVVVDVSHEDPEVKFNWYVDGVEVHNAKTKPREEQYN
STYRVVSVLTVLHQDWLNGKEYKCKVSNKALPAPIEKTI
SKAKGQPREPQVYTLPPSRDELTKNQVSLLCLVKGFYPS
DIAVEWESNGQPENNYKTTPPVLDSDGSFFLHSKLTVDK
SRWQQGNVFSCSVMHEALHNHYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline.
1.2. Cloning of 1.3 proBiTE
To generate the construct of 1.3 proBiTE, the following primers were used:

E005-1.3-HC-mask-FP-1,
5′-ATTAGCGGAACTGGTGGAGGCTCTGGCGGAGGCTCAGGTGGTGGCTC
AGGACTGAGCGGAAGA-3′;
E005-1.3-HC-mask-FP-2,
5′-CAGGATGGCAATGAAGAAATGGGTGGCATCACCCAGACTCCGTATAA
GGTGAGCATTAGCGGAACT-3′;
E005-1.3-HC-mask-FP-3,
5′-GCACTGCTCTGTTGCCTGGTCCTCCTGACTGGGGTGAGGGCCCAGGA
TGGCAATG-3′;
pBY-Fc-RP-Xba I,
5′-ATCGTCTAGAATCACTTGCCGGGGCTCAGGCT-3′;
pBY-SP-Not I,
5′-ATAAGAATGCGGCCGCAAACTACAAGACAGACTTGCAAAAGAAGGCA
TGCACAGCTCAGCACTGCTCTGTTGC-3′.

For cloning of 1.3 proBiTE (FIG. 2), the mask gene fragment (part of CD3 epsilon subunit of T-cell receptor complex, Homo sapiens) of heavy chain was amplified by overlapping PCR with primers E005-1.3-HC-mask-FP-1/-2/-3 and pBY-Fc-RP-Xba I. The PCR product was linked to a gene fragment encoding a signal leader peptide by overlapping PCR with primers pBY-Fc-RP-Xba I and pBY—SP-Not I. The product was digested with Xba I and Not I restriction sites, and cloned into the pcDNA3.4 vector. The light chain gene fragment was consistent with the light chain of 1.1 proBiTE.
The full-length amino acid sequences of 1.3 proBiTE are shown as follows.

Heavy chain
(SEQ ID NO: 53)
QDGNEEMGGITQTPYKVSISGTGGGSGGGSGGGSG [symbol]
GGSEVQLVESGGGLVQPGGSLRLSCAASGFTFNTYAMNWVRQA
PGKGLEWVARIRSKYNNYATYYADSVKGRFTISRDDSKNTLYL
QMNSLRAEDTAVYYCARHGNFGSSYVSYFAYWGQGTTVTVSS
GGGSSGGGGSGGGGSEVQLVETGGGVVQPGRSLRL
SCAASGFTFSSYAMHWVRQAPGKGLEWVAVISYDG
SNKYYADSVKGRFTISRDNSKNTLYLQMNSLRAED
TAVYYCARGYVARGDFDYWGQGTLVTVSS
ASTKGPSVFPLAPSSKSTSGGTAALGCLVKDYFPEPVTVSWNS
GALTSGVHTFPAVLQSSGLYSLSSVVTVPSSSLGTQTYICNVN
HKPSNTKVDKKVEPKSCPPCPAPELLGGPSVFLFPPKPKDTLM
ISRTPEVTCVVVDVSHEDPEVKFNWYVDGVEVHNAKTKPREEQ
YNSTYRVVSVLTVLHQDWLNGKEYKCKVSNKALPAPIEKTISK
AKGQPREPQVYTLPPSRDELTKNQVSLLCLVKGFYPSDIAVEW
ESNGQPENNYKTTPPVLDSDGSFFLHSKLTVDKSRWQQGNVFS
CSVMHEALHNHYTQKSLSLSPGK,

wherein anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline; the mask peptide is shown as bold and double underline; and the protease cleavage site is shown as bold and wavy line.

Light chain
(SEQ ID NO: 50)
EIVVTQSPATLSVSPGERATLSCRS
STGAVTTSNYANWVQQKPGQAPRGL
IGGANKRAPGVPARFSGSLSGDEAT
LTISSLQSEDFAVYYCALWYSNLWV
FGQGTKLEIKGGGGSGGGGSGGGGS
DVVMTQSPSTLSASVGDRVTITCRAS
QSMSRWLAWYQQKPGKGPKLLI
YATSTLQSGVPSRFSGSGSGTEFTLT
ISSLQPEDFATYYCQQLDSFPFSFT
FGPGTKVDLKRTVAAPSVFIFPPSD
EQLKSGTASVVCLLNNFYPREAKVQ
WKVDNALQSGNSQESVTEQDSKDST
YSLSSTLTLSKADYEKHKVYACEVT
HQGLSSPVTKSFNRGECPPCPAPEL
LGGPSVFLFPPKPKDTLMISRTPEV
TCVVVDVSHEDPEVKFNWYVDGVEV
HNAKTKPREEQYNSTYRVVSVLTVL
HQDWLNGKEYKCKVSNKALPAPIEK
TISKAKGQPREPQVYTLPPSRDELT
KNQVSLLCLVKGFYPSDIAVEWESN
GQPENNYKTTPPVLDSDGSFFLHSK
LTVDKSRWQQGNVFSCSVMHEALHN
HYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline.
1.3. Cloning of 1.4/1.4.1/1.4.3 proBiTEs
In order to increase blocking effect, several sets of proBiTEs whose linker comprise two tandem mask peptides were constructed. The following primers were used:

E005-1.4-HC-FP,
5′-GGAAATGGGTGGTGGCGGCGGCTCAGGAGGAGGATCAGGCGGA-3′;
E005-1.4-HC-RP,
5′-TCATTGCCATCTTGGCTGCCACCACCGATGCCGCCCATCTCTTCGTT
TCC-3′;
E005-1.4.1-HC-FP,
5′-GGTGGTGGCAGCCAGGACGGAAACGAAGAGATGGGCGGCATC-3′;
E005-1.4.1-HC-RP,
5′-CATTTCCTCATTGCCATCTTGGGCCCTCACCCCAGTCAGGAGGACC
A-3′;
E005-1.4.3-HC-FP,
5′-GGAGGATCAGGCGGAGGAAGCGGCCTGAGCGGAAGAAGCGA-3′;
E005-1.4.3-HC-RP,
5′-GCCACCACCCATTTCCTCATT-3′.

For cloning of 1.4 and 1.4.1 proBiTEs, a (Gly₃Ser)₃G linker was used to connect mask2+1 peptide and mask1+3 peptide at the 5′ end of the anti-CD3 Fv region of the heavy chain, respectively. On the basis of 1.1 proBiTE, the tandemed mask gene fragments were amplified by inversing PCR with primers E005-1.4/1.4.1-HC-FP and E005-1.4/1.4.1-HC-RP. For cloning of 1.4.3 proBiTE, the length of the linker between the uPA cleavable site and the anti-CD3 Fv region was modified. Based on 1.4 proBiTE, the modified linker was amplified by inversing PCR with primers E005-1.4.3-HC-FP and E005-1.4.3-HC-RP (FIG. 2).
The full-length amino acid sequences of 1.4, 1.4.1 and 1.4.3 proBiTEs are shown as follows.
1.4 proBiTE

Heavy chain
(SEQ ID NO: 54)
QDGNEEMGGIGGGSQDGNEEM
GGGGGSGGGSGGGSG [symbol]
GGSEVQLVESGGGLVQPGGSLRLS
CAASGFTFNTAMNWVRQAPGKGLE
WVARIRSKYNNYATYYADSVKGRF
TISRDDSKNTLYLQMNSLRAEDTA
VYYCARHGNFGSSYVSYFAYWGQG
TTVTVSSGGGSSGGGGSGGGGS
EVQLVETGGGVVQPGRSLRLSC
AASGFTFSSYAMHWVRQAPGKG
LEWVAVISYDGSNKYYADSVKG
RFTISRDNSKNTLYLQMNSLRA
EDTAVYYCARGVYARGDFDYWG
QGTLVTVSS
ASTKGPSVFPLAPSSKSTSGGTAA
LGCLVKDYFPEPVTVSWNSGALTS
GVHTFPAVLQSSGLYSLSSVVTVP
SSSLGTQTYICNVNHKPSNTKVDK
KVEPKSCPPCPAPELLGGPSVFLF
PPKPKDTLMISRTPEVTCVVVDVS
HEDPEVKFNWYVDGVEVHNAKTKP
REEQYNSTYRVVSVLTVLHQDWLN
GKEYKCKVSNKALPAPIEKTISKA
KGQPREPQVYTLPPSRDELTKNQV
SLLCLVKGFYPSDIAVEWESNGQP
ENNYKTTPPVLDSDGSFFLHSKLT
VDKSRWQQGNVFSCSVMHEALHNH
YTQKSLSLSPGK,

wherein anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline; the mask peptide is shown as bold and double underline; and the protease cleavage site is shown as bold and wavy line.

Light chain
(SEQ ID NO: 50)
EIVVTQSPATLSVSPGERATLSCRSSTGAVTTSNYANWVQQKP
GQAPRGLIGGANKRAPGVPARFSGSLSGDEATLTISSLQSEDF
AVYYCALWYSNLWVFGQGTKLEIKGGGGSGGGGSGGGGS
DVVMTQSPSTLSASVGDRVTITCRASQSMSRW
LAWYQQKPGKGPKLLIYATSTLQSGVPSRFSG
SGSGTEFTLTISSLQPEDFATYYCQQLDSFP
FSFTFGPGTKVDLKRTVAAPSVFIFPPSDEQ
LKSGTASVVCLLNNFYPREAKVQWKVDNALQSGNSQESVTEQ
DSKDSTYSLSSTLTLSKADYEKHKVYACEVTHQGLSSPVTKS
FNRGECPPCPAPELLGGPSVFLFPPKPKDTLMISRTPEVTCV
VVDVSHEDPEVKFNWYVDGVEVHNAKTKPREEQYNSTYRVVS
VLTVLHQDWLNGKEYKCKVSNKALPAPIEKTISKAKGQPREP
QVYTLPPSRDELTKNQVSLLCLVKGFYPSDIAVEWESNGQPE
NNYKTTPPVLDSDGSFFLHSKLTVDKSRWQQGNVFSCSVMHE
ALHNHYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline.
1.4.1 proBiTE

Heavy chain
(SEQ ID NO: 55)
QDGNEEMGGGSQDGNEEMGGITQTPYGGGSGGGSGGGSG [symbol]
GGSEVQLVESGGGLVQPGGSLRLSCAASGFT
FNTYAMNWVRQAPGKGLEWVARIRSKYNNYATYYADSVK
GRFTISRDDSKNTLYLQMNSLRAEDTAVYYCARHGNFGS
SYVSYFAYWGQGTTVTVSSGGGSSGGGGSGGGGS
EVQLVETGGGVVQPGRSLRLSCAAS
GFTFSSYAMHWVRQAPGKGLEWVAV
ISYDGSNKYYADSVKGRFTISRDNS
KNTLYLQMNSLRAEDTAVYYCARGY
VARGDFDYWGQGTLVTVSS
ASTKGPSVFPLAPSSKSTSGGTAALGCLVKDYFPEPVTV
SWNSGALTSGVHTFPAVLQSSGLYSLSSVVTVPSSSLGT
QTYICNVNHKPSNTKVDKKVEPKSCPPCPAPELLGGPSV
FLFPPKPKDTLMISRTPEVTCVVVDVSHEDPEVKFNWYV
DGVEVHNAKTKPREEQYNSTYRVVSVLTVLHQDWLNGKE
YKCKVSNKALPAPIEKTISKAKGQPREPQVYTLPPSRDE
LTKNQVSLLCLVKGFYPSDIAVEWESNGQPENNYKTTPP
VLDSDGSFFLHSKLTVDKSRWQQGNVFSCSVMHEALHNH
YTQKSLSLSPGK,

wherein anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline; the mask peptide is shown as bold and double underline; and the protease cleavage site is shown as bold and wavy line.

Light chain
(SEQ ID NO: 50)
EIVVTQSPATLSVSPGERATLSCRSSTGAVTTSNYANWVQQ
KPGQAPRGLIGGANKRAPGVPARFSGSLSGDEATLTISSLQ
SEDFAVYYCALWYSNLWVFGQGTKLEIKGGGGSGGGGSGGGGS
DVVMTQSPSTLSASVGDRVTITCRAS
QSMSRWLAWYQQKPGKGPKLLIYATS
TLQSGVPSRFSGSGSGTEFTLTISSLQPE
DFATYYCQQLDSFPFSFTFGPGTKVDLK
RTVAAPSVFIFPPSDEQLKSGTASVVCLLNNFYPREAKVQWKV
DNALQSGNSQESVTEQDSKDSTYSLSSTLTLSKADYEKHKVYA
CEVTHQGLSSPVTKSFNRGECPPCPAPELLGGPSVFLFPPKPK
DTLMISRTPEVTCVVVDVSHEDPEVKFNWYVDGVEVHNAKTKP
REEQYNSTYRVVSVLTVLHQDWLNGKEYKCKVSNKALPAPIEK
TISKAKGQPREPQVYTLPPSRDELTKNQVSLLCLVKGFYPSDI
AVEWESNGQPENNYKTTPPVLDSDGSFFLHSKLTVDKSRWQQG
NVFSCSVMHEALHNHYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline.
1.4.3 proBiTE

Heavy chain
(SEQ ID NO: 56)
QDGNEEMGGIGGGSQDGNEEM
GGGGGSGGGSG [symbol]
GGSEVQLVESGGGLVQPGGSL
RLSCAASGFTFNTYAMNWVRQ
APGKGLEWVARIRSKYNNYAT
YYADSVKGRFTISRDDSKNTL
YLQMNSLRAEDTAVYYCARH
GNFGSSYVSYFAYWGQGTTV
TVSSGGGSSGGGGSGGGGS
EVQLVETGGGVVQPGRSLRLS
CAASGFTFSSYAMHWVRQAPG
KGLEWVAVISYDGSNKYYADS
VKGRFTISRDNSKNTLYLQMN
SLRAEDTAVYYCARGYVARGD
FDYWGQGTLVTVSSASTKGPS
VFPLAPSSKSTSGGTAALGCL
VKDYFPEPVTVSWNSGALTSG
VHTFPAVLQSSGLYSLSSVVT
VPSSSLGTQTYICNVNHKPSN
TKVDKKVEPKSCPPCPAPELL
GGPSVFLFPPKPKDTLMISRT
PEVTCVVVDVSHEDPEVKFNW
YVDGVEVHNAKTKPREEQYNS
TYRVVSVLTVLHQDWLNGKEY
KCKVSNKALPAPIEKTISKAK
GQPREPQVYTLPPSRDELTKN
QVSLLCLVKGFYPSDIAVEWE
SNGQPENNYKTTPPVLDSDGS
FFLHSKLTVDKSRWQQGNVFS
CSVMHEALHNHYTQKSLSLSP
GK,

wherein anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline; the mask peptide is shown as bold and double underline; and the protease cleavage site is shown as bold and wavy line.

Light chain
(SEQ ID NO: 50)
EIVVTQSPATLSVSPGERATLSC
RSSTGAVTTSNYANWVQQKPGQA
PRGLIGGANKRAPGVPARFSGSL
SGDEATLTISSLQSEDFAVYYCA
LWYSNLWVFGQGTKLEIKGGGGS
GGGGSGGGGS
DVVMTQSPSTLSASVGDRVTITC
RASQSMSRWLAWYQQKPGKGPKL
LIYATSTLQSGVPSRFSGSGSGT
EFTLTISSLQPEDFATYYCQQLD
SFPFSFTFGPGTKVDLK
RTVAAPSVFIFPPSDEQLKSGTA
SVVCLLNNFYPREAKVQWKVDNA
LQSGNSQESVTEQDSKDSTYSLS
STLTLSKADYEKHKVYACEVTHQ
GLSSPVTKSFNRGECPPCPAPEL
LGGPSVFLFPPKPKDTLMISRTP
EVTCVVVDVSHEDPEVKFNWYVD
GVEVHNAKTKPREEQYNSTYRVV
SVLTVLHQDWLNGKEYKCKVSNK
ALPAPIEKTISKAKGQPREPQVY
TLPPSRDELTKNQVSLLCLVKGF
YPSDIAVEWESNGQPENNYKTTP
PVLDSDGSFFLHSKLTVDKSRWQ
QGNVFSCSVMHEALHNHYTQKSL
SLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline.
1.4. Cloning of 1.5 proBiTE
To improve cleavability, 1.5 proBiTE containing two cleavage sites was constructed (FIG. 3). The following primers were used:

E005-1.5-LC-CD3VL-FP,
5′-AGGAAGCGAGATTGTCGTCACTCAGAGTCCCGCCACACTGAGTGT
GAG-3′;
E005-1.5-LC-UPA-RP,
5′-AGTGACGACAATCTCGCTTCCTCCGTGGTTGTCGCTT-3′;
E005-1.5-LC-UPA-RP-1,
5′-GGGACTCTGAGTGACGACAATCTCGCTTCCTCCGTGGTTGTCGCT
TCTTCCGCTCA-3′.

For cloning of 1.5 proBiTE, the uPA gene fragment of light chain was amplified by overlapping PCR with primers E005-1.5-LC-CD3VL-FP and E005-1.5-LC-UPA-RP, and the PCR product continued to amplify by overlapping PCR with primers E005-1.5-LC-UPA-RP-1 and E005-1.5-LC-UPA-RP. The PCR product was linked to a gene fragment encoding a signal leader peptide by overlapping PCR with primers pBY-Fc-RP-Xba I and pBY-SP-Not I. The product was digested with Xba I and Not I restriction sites, and cloned into the pcDNA3.4 vector.
The full-length amino acid sequences of 1.5 proBiTE are shown as follows.

Heavy chain
(SEQ ID NO: 52)

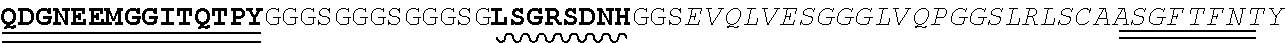

AMNWVRQAPGKGLEWVARIRSKYNNYATYYADSVKGRFTISRDDSKNTLYLQMNSLRAEDTAVYYCARHGNEGSS
YVSYFAYWGQGTTVTVSSGGGSSGGGGSGGGGSEVQLVETGGGVVQPGRSLRLSCAASGFTESSYAMHW
VRQAPGKGLEWVAVISYDGSNKYYADSVKGRFTISRDNSKNTLYLQMNSLRAEDTAVYYCARGYVA
RGDFDYWGQGTLVTVSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCPPCPAPELLGGPSVFLFPPKPKDTLMISRTP
EVTCVVVDVSHEDPEVKENWYVDGVEVHNAKTKPREEQYNSTYRVVSVLTVLHQDWLNGKEYKCKVSNKALPAP
IEKTISKAKGQPREPQVYTLPPSRDELTKNOVSLLCLVKGFYPSDIAVEWESNGQPENNYKTTPPVLDSDGSFFLHS
KLTVDKSRWQQGNVESCSVMHEALHNHYTQKSLSLSPGK

wherein anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline; the mask peptide is shown as bold and double underline; and the protease cleavage site is shown as bold and wavy line.

Light chain
(SEQ ID NO: 57)

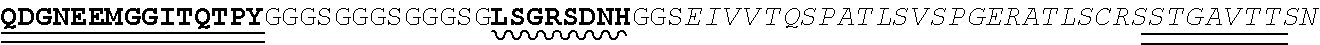

YANWVQQKPGQAPRGLIGGANKRAPGVPARFSGSLSGDEATLTISSLQSEDFAVYYCALWYSNLWVFGQGTKLEI
KGGGGSGGGGSGGGGSDVVMTQSPSTLSASVGDRVTITCRASQSMSRWLAWYQQKPGKGPKLLIYAT
STLQSGVPSRESGSGSGTEFTLTISSLQPEDFATYYCQQLDSFPESFTFGPGTKVDLKRTVAAPSVFIFPPS
DEQLKSGTASVVCLLNNFYPREAKVOWKVDNALOSGNSOESVTEODSKDSTYSLSSTLTLSKADYEKHKVYACEVT
HQGLSSPVTKSFNRGECPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLPPSRDELTK
NQVSLLCLVKGFYPSDIAVEWESNGQPENNYKTTPPVLDSDGSFFLHSKLTVDKSRWQQGNVFSCSVMHEALHN
HYTQKSLSLSPGK,

wherein anti-CD3 VL is shown as italics and LCDRs 1-3 therein is double underlined; anti-EGFR VL is shown as bold and LCDRs 1-3 therein is double underlined; CL-CH2-CH3 is shown as italics and double underline; the mask peptide is shown as bold and double underline; and the protease cleavage site is shown as bold and wavy line.
1.5. Cloning of 1.6/1.6.1/1.7/1.7.1 proBiTEs
To increase the affinity for a tumor antigen (e.g. EGFR), four more constructs 1.6, 1.6.1, 1.7, and 1.7.1 proBiTEs were generated (FIG. 3), and the full-length gene sequences of them were synthesized by Biointron (Shanghai). The heavy chain of 1.6.1 proBiTE contains two tandem mask peptides, while the light chain was designed to possess a Single-Chain Fragment Variable (scFv) of anti-EGFR antibody, which was fused via the (G₄S)₄ linker to the N terminus of the VL-CL. The heavy chain of 1.7 and 1.7.1 bivalent proBiTE also contain two tandem mask peptides at the N terminus of the VH-CH1, while the heavy and light chains were designed to possess two Single-Chain Fragment Variable (scFv) of anti-EGFR antibodies, which were fused via the (G₄S)₃ and (G₄S)₄ linkers to the C terminus of the CH3 domains, respectively.
The full-length amino acid sequences of 1.6, 1.6.1, 1.7, and 1.7.1 proBiTEs are shown as follows.
1.6 proBiTE

Heavy chain
(SEQ ID NO: 58)

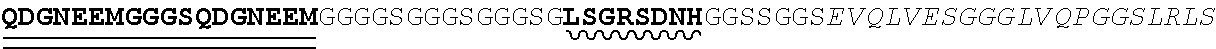

CAASGFTENTYAMNWVRQAPGKGLEWVARIRSKYNNYATYYADSVKGRFTISRDDSKNTLYLQMNSLRAEDTAVY
YCARHGNFGSSYVSYFAYWGQGTTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCPPCPAPELLGGPSVELFPPKPK
DTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLLCLVKGFYPSDIAVEWESNGQPENNYKTTPPVLD
SDGSFFLHSKLTVDKSRWQQGNVESCSVMHEALHNHYTQKSLSLSPGKGGGGSGGGGSGGGGSEVQLVETG
GGVVQPGRSLRLSCAASGFTFSSYAMHWVRQAPGKGLEWVAVISYDGSNKYYADSVKGRFTISRDNS
KNTLYLQMNSLRAEDTAVYYCARGYVARGDFDYWGQGTLVTVSS,

wherein anti-CD3 VH is shown as italics and HCDRs 1-3 therein is double underlined; CH1-CH2-CH3 is shown as italics and double underline; anti-EGFR VH is shown as bold and HCDRs 1-3 therein is double underlined; the mask peptide is shown as bold and double underline; and the protease cleavage site is shown as bold and wavy line.

Light chain
(SEQ ID NO: 59)
EIVVTQSPATLSVSPGERATLSCRSSTGAVTTSNYANWVQQKPGQAPRGLIGGANKRAPGVPARFSGSLSGDEATLT
ISSLQSEDFAVYYCALWYSNLWVFGQGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGECPPCPAPELLGGP
SVFLFPPKPKDTLMISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLLCLVKGFYPSDIAVEWESNGQPEN
NYKTTPPVLDSDGSFFLHSKLTVDKSRWQQGNVFSCSVMHEALHNHYTQKSLSLSPGKGGGGSGGGGSGGGGS
DVVMTQSPSTLSASVGDRVTITCRAS [symbol] LAWYQQKPGKGPKLLIY [symbol] TLQSGVPSRFSGSGS
GTEFTLTISSLQPEDFATYYC [symbol] FGPGTKVDLK,

wherein anti-CD3 VL is shown as italics and LCDRs 1-3 therein is double underlined; CL-CH2-CH3 is shown as italics and double underline; and anti-EGFR VL is shown as bold and LCDRs 1-3 therein is double underlined.
1.6.1 proBiTE

Heavy chain
(SEQ ID NO: 60)

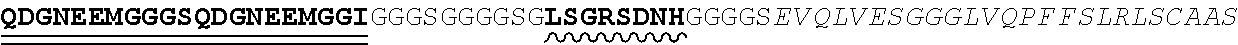

GFTENTYAMNWVRQAPGKGLEWVARIRSKYNNYATYYADSVKGRFTISRDDSKNTLYLQMNSLRAEDTAVYYCAR
HGNFGSSYVSYFAYWGQGTTVTVSSASTKGPSVFPLAPSSKSTSGGTAALGCLVKDYFPEPVTVSWNSGALTSGVH
TFPAVLOSSGLYSLSSVVTVPSSSLGTQTYICNVNHKPSNTKVDKKVEPKSCPPCPAPELLGGPSVFLFPPKPKDTL
MISRTPEVTCVVVDVSHEDPEVKENWYVDGVEVHNAKTKPREEQYNSTYRVVSVLTVLHODWLNGKEYKCKVSN
KALPAPIEKTISKAKGQPREPQVYTLPPSRDELTKNOVSLLCLVKGFYPSDIAVEWESNGQPENNYKTTPPVLDSD
GSFFLHSKLTVDKSRWOOGNVESCSVMHEALHNHYTOKSLSLSPGK,

wherein anti-CD3 VH is shown as italics and HCDRs 1-3 therein is double underlined; CH1-CH2-CH3 is shown as italics and double underline; the mask peptide is shown as bold and double underline; and the protease cleavage site is shown as bold and wavy line.

Light chain
(SEQ ID NO: 61)
EVQLVETGGGVVQPGRSLRLSCAAS [symbol] MHWVRQAPGKGLEWVAV [symbol] YYADSVKG
RFTISRDNSKNTLYLQMNSLRAEDTAVYYC [symbol] WGQGTLVTVSSGGGGSGGGGSGGG
GSGGGGSDVVMTQSPSTLSASVGDRVTITCRASQSMSRWLAWYQQKPGKGPKLLIYATSTLQSGVPSRFSGSGSGT
EFTLTISSLQPEDFATYYCQQLDSFPFSFTFGPGTKVDLKGGGGSGGGGSGGGGSGGGGSEIVVTQSPATLSVS
PGERATLSCRSS [symbol] ANWVQQKPGQAPRGLIG [symbol] KRAPGVPARFSGSLSGDEATLTISSLQSEDF
AVYYC [symbol] FGQGTKLEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGECPPCPAPELLGGPSVFLFPPK
PKDTLMISRTPEVTCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLLCLVKGFYPSDIAVEWESNGQPENNYKTTPP
VLDSDGSFFLHSKLTVDKSRWQQGNVFSCSVMHEALHNHYTQKSLSLSPGK,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline.
1.7 proBiTE

Heavy chain
(SEQ ID NO: 62)

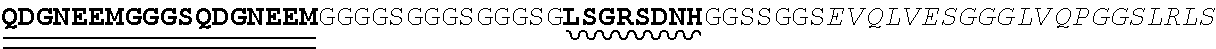

CAASGFTENTYAMNWVRQAPGKGLEWVARIRSKYNNYATYYADSVKGRFTISRDDSKNTLYLQMNSLRAEDTAVY
YCARHGNFGSSYVSYFAYWGQGTTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCPPCPAPELLGGPSVFLFPPKPK
DTLMISRTPEVTCVVVDVSHEDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLLCLVKGFYPSDIAVEWESNGOPENNYKTTPPVLD
SDGSFFLHSKLTVDKSRWQQGNVFSCSVMHEALHNHYTQKSLSLSPGKGGGGSGGGGSGGGGSEVQLVETG
GGVVQPGRSLRLSCAASGFTESSYAMHWVRQAPGKGLEWVAVISYDGSNKYYADSVKGRFTISRDNS
KNTLYLQMNSLRAEDTAVYYCARGYVARGDFDYWGQGTLVTVSSGGGGSGGGGSGGGGSDVVMTQS
PSTLSASVGDRVTITCRAS [symbol] LAWYQQKPGKGPKLLIY [symbol] TLQSGVPSRFSGSGSGTEFTLTISSLQP
EDFATYYC [symbol] FGPGTKVDLK,

wherein the mask peptide is shown as bold and double underline; the protease cleavage site is shown as bold and wavy line; anti-CD3 VH is shown as italics and HCDRs 1-3 therein is double underlined; CH1-CH2-CH3 is shown as italics and double underline; anti-EGFR VH is shown as bold and HCDRs 1-3 therein is double underlined; anti-EGFR VL is shown as hold and italics and LCDRs 1-3 therein is double underlined.

Light chain
(SEQ ID NO: 63)
EIVVTQSPATLSVSPGERATLSCRSSTGAVTTSNYANWVQQKPGQAPRGLIGGANKRAPGVPARFSGSLSGDEATLT
ISSLQSEDFAVYYCALWYSNLWVFGQGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGECPPCPAPELLGGP
SVFLFPPKPKDTLMISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLLCLVKGFYPSDIAVEWESNGQPEN
NYKTTPPVLDSDGSFFLHSKLTVDKSRWQQGNVFSCSVMHEALHNHYTQKSLSLSPGKGGGGSGGGGSGGGGS
EVQLVETGGGVVQPGRSLRLSCAAS [symbol] MHWVRQAPGKGLEWVAV [symbol] YYADSVKG
RFTISRDNSKNTLYLQMNSLRAEDTAVYYC [symbol] WGQGTLVTVSSGGGGSGGGGSGGG
GSDVVMTQSPSTLSASVGDRVTITCRAS [symbol] LAWYQQKPGKGPKLLIY [symbol] TLQSGVPSRFSGSGSGTE
FTLTISSLQPEDFATYYC [symbol] FGPGTKVDLK,

wherein anti-CD3 VL is shown as italics and HCDRs 1-3 therein is double underlined; CL-CH2-CH3 is shown as italics and double underline; anti-EGFR VH is shown as bold and HCDRs 1-3 therein is double underlined; anti-EGFR VL is shown as hold and italics and LCDRs 1-3 therein is double underlined.
1.7.1 proBiTE

Heavy chain
(SEQ ID NO: 64)

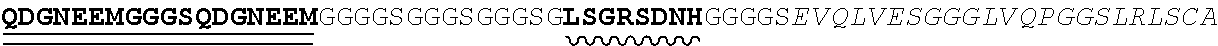

ASGFTENTYAMNWVRQAPGKGLEWVARIRSKYNNYATYYADSVKGRFTISRDDSKNTLYLQMNSLRAEDTAVYYC
ARHGNFGSSYVSYFAYWGQGTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCPPCPAPELLGGPSVFLFPPKPKD
TLMISRTPEVTCVVVDVSHEDPEVKENWYVDGVEVHNAKTKPREEQYNSTYRVVSVLTVLHQDWLNGKEYKCKVS
NKALPAPIEKTISKAKGQPREPQVYTLPPSRDELTKNQVSLLCLVKGFYPSDIAVEWESNGQPENNYKTTPPVLDS
DGSFFLHSKLTVDKSRWQQGNVFSCSVMHEALHNHYTQKSLSLSPGKGGGGSGGGGSGGGGSGGGGSEVQL
VETGGGVVQPGRSLRLSCAASGFTESSYAMHWVRQAPGKGLEWVAVISYDGSNKYYADSVKGRFTI
SRDNSKNTLYLQMNSLRAEDTAVYYCARGYVARGDEDYWGQGTLVTVSSGGGGSGGGGSGGGGSGG
GGSDVVMTQSPSTLSASVGDRVTITCRAS [symbol] LAWYQQKPGKGPKLLIY [symbol] TLQSGVPSRFSGSGSGT
EFTLTISSLQPEDFATYYC [symbol] FGPGTKVDLK,

wherein the mask peptide is shown as bold and double underline; the protease cleavage site is shown as bold and wavy line; anti-CD3 VH is shown as italics and HCDRs 1-3 therein is double underlined; CH1-CH2-CH3 is shown as italics and double underline; anti-EGFR VH is shown as bold and HCDRs 1-3 therein is double underlined; anti-EGFR VL is shown as hold and italics and LCDRs 1-3 therein is double underlined.

Light chain
(SEQ ID NO: 65)
EIVVTQSPATLSVSPGERATLSCRSSTGAVTTSNYANWVQQKPGQAPRGLIGGANKRAPGVPARFSGSLSGDEATLT
ISSLQSEDFAVYYCALWYSNLWVFGQGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGECPPCPAPELLGGP
SVFLFPPKPKDTLMISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLLCLVKGFYPSDIAVEWESNGQPEN
NYKTTPPVLDSDGSFFLHSKLTVDKSRWQQGNVFSCSVMHEALHNHYTQKSLSLSPGKGGGGSGGGGSGGGGS
GGGGSEVQLVETGGGVVQPGRSLRLSCAAS [symbol] MHWVRQAPGKGLEWVAV [symbol] YYA
DSVKGRFTISRDNSKNTLYLQMNSLRAEDTAVYYC [symbol] WGQGTLVTVSSGGGGSGGG
GSGGGGSGGGGSDVVMTQSPSTLSASVGDRVTITCRAS [symbol] LAWYQQKPGKGPKLLIY [symbol] TLQSGVP
SRFSGSGSGTEFTLTISSLQPEDFATYYC [symbol] FGPGTKVDLK,

wherein anti-CD3 VL is shown as italics and HCDRs 1-3 therein is double underlined; CL-CH2-CH3 is shown as italics and double underline; anti-EGFR VH is shown as bold and HCDRs 1-3 therein is double underlined; anti-EGFR VL is shown as hold and italics and LCDRs 1-3 therein is double underlined.

### 1.6. Protein Expression and Purification

Bispecific antibodies were expressed in either 293FS or CHO-S cells. The plasmids and transfection agent PEI were mixed at ratio 1:3 and then added into 293FS cell culture dropwise. And total plasmid DNA in the range of 1.0 μg per mL to be transfected in CHO-S cell culture dropwise. The cells were continued to grow for 5-7 days after transfection. The cell culture was harvested by centrifugation at 8000 rpm for 20 min. The culture supernatant containing target proteins were loaded onto Protein A Sepharose 4 Fast Flow column (GE Healthcare). The subsequent purification was carried out according to the manufacturer's instructions.

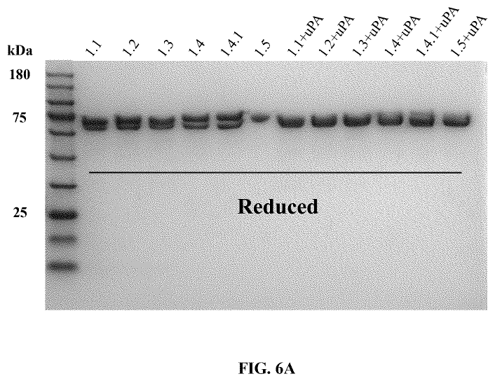

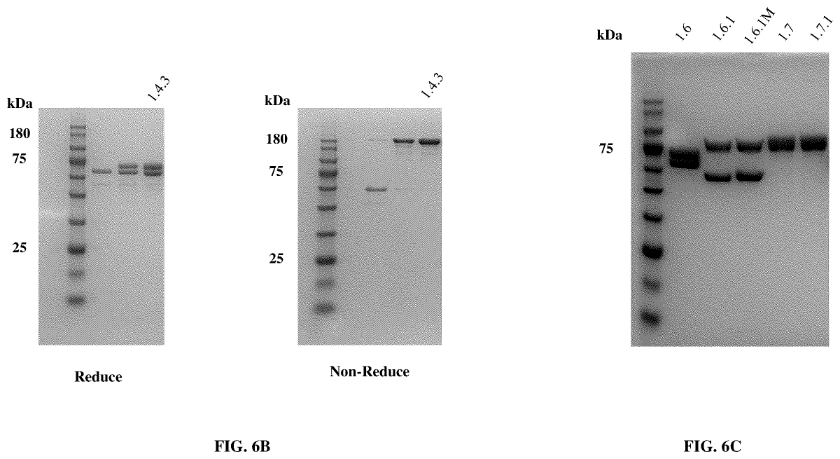

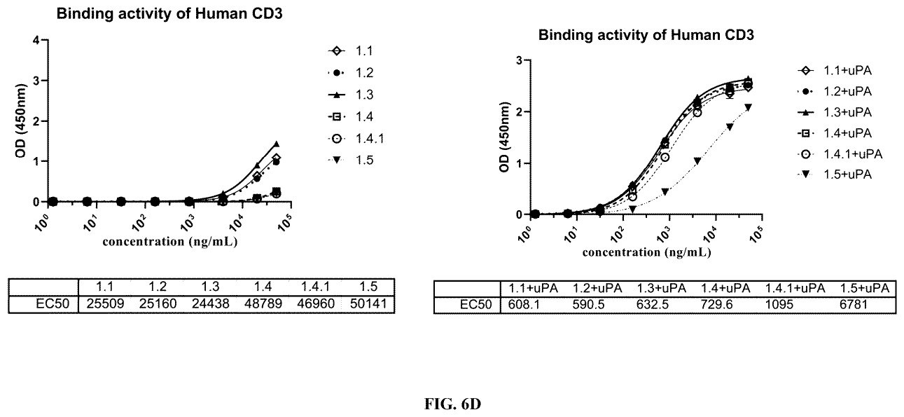

The results showed that all the bispecific antibodies were well expressed in transiently transfected either 293 free style (293FS) or CHO-S cells and secreted into the culture supernatants. On a non-reducing SDS-PAGE, a vast majority of the purified antibodies migrated as a heterodimer with apparent molecular weight (aMW) of approximately 150 kDa (FIGS. 6A-6C). On a reducing SDS-PAGE, the two polypeptide chains of 1.1-1.4.1 proBiTE were well separated while those of 1.5 proBiTE overlapped with each other with apparent molecular weight of approximately 75 kDa.

### Example 2. Blocking Effect on Human CD3 and Binding Affinity to Human EGFR of proBiTEs

To determine the binding affinity of proBiTEs to CD3 and EGFR, ELISA was performed according to standard protocols. Briefly, human antigen CD3 and EGFR were coated on Corning EIA/RIA high-binding 96-well plates (Corning Inc.) at 100 ng per well overnight at 4° C. and blocked with 3% nonfat milk in PBS (pH7.4). For cleavage with urokinase (uPA), 30 μg antibodies were mixed with or without 0.3 μg recombinant human uPA (50 μg/mL) (Sino Biological) in PBS (pH7.4) and incubated at room temperature for 4 hour. Then the mixture with or without uPA was diluted in a fivefold gradient and incubated at room temperature for 2 h. The plates were washed with PBS containing 1% nonfat milk. Bound antibodies were detected by HRP-conjugated streptavidin (Sigma). The assay was developed at room temperature with TMB substrate (Solarbio) and monitored at 450 nm with a microplate reader. The half-maximal binding (EC₅₀) was calculated by fitting the data to the Langmuir adsorption isotherm. The results were shown in FIGS. 6D-9B.

FIG. 6D shows that 1.1, 1.2, 1.3, 1.4, 1.4.1 and 1.5 proBiTEs have strong blocking effect on CD3 binding, and the binding affinity of these proBiTEs against CD3 is recovered after uPA digestion. The binding affinity of 1.5 proBiTE against CD3 after uPA digestion is slightly weaker than other proBiTEs, which is probably resulted from incomplete uPA digestion of 1.5 proBiTE that has a uPA protease cleavage site at both of heavy and light chains.

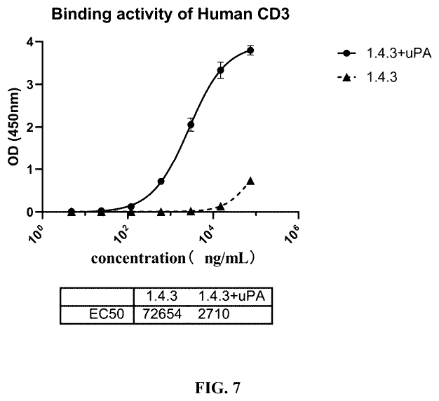

FIG. 7 shows that 1.4.3 proBiTE has strong blocking effect on human CD3 antigen, and its binding affinity against CD3 is recovered after uPA digestion.

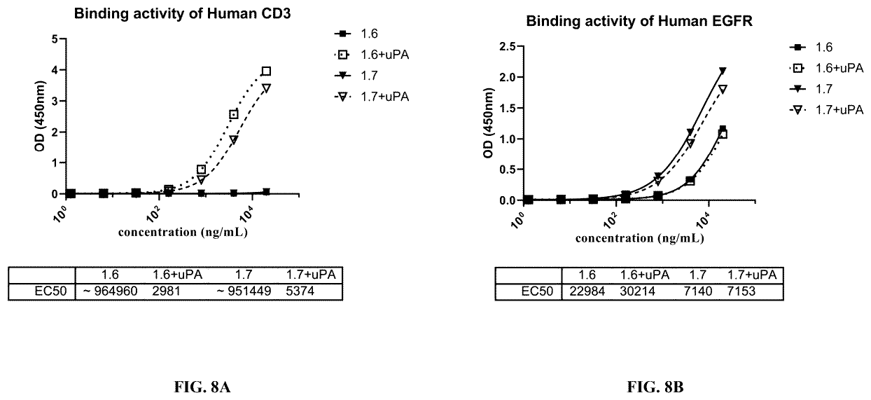

FIG. 8A shows that 1.6 and 1.7 proBiTEs have strong blocking effect on human CD3 antigen, and the binding affinity of these proBiTEs against CD3 is recovered after uPA digestion.

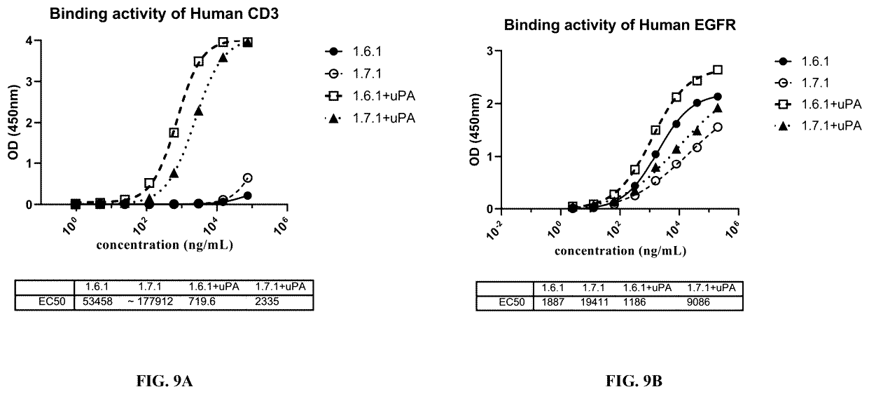

FIG. 9A shows that 1.6.1 and 1.7.1 proBiTEs have strong blocking effect on human CD3 antigen, and the binding affinity of these proBiTEs against CD3 is recovered after uPA digestion.

The results indicated that the proBiTEs of the invention, particularly 1.6.1, 1.7, 1.7.1, 1.4, 1.4.1, 1.4.3 proBiTEs, have strong blocking effect on human CD3 antigen, and the binding affinity of these proBiTEs against CD3 is recovered after incubation with uPA. This suggests that the configurational blocking efficiency of two mask peptides in series is higher than that of a single mask peptide.

FIGS. 8B and 9B indicates that there was no significant difference in the binding activity of the proBiTEs of the invention to EGFR before and after uPA digestion.

### Example 3. Construction and Initial Characterization of CD3×EGFR Variant proBiTEs

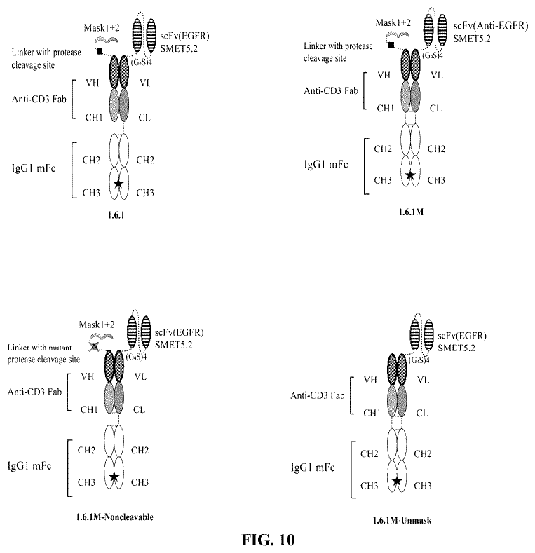

Based on 1.4.3 and 1.6.1 proBiTEs, the human IgG1 Fc-engineered variant proBiTEs (FIGS. 10-12) were designed to reduce antibody-dependent cell-mediated cytotoxicity (ADCC).

3.1. Cloning of 1.6.1M proBiTE, 1.6.1M-Unmask iBiTE and 1.6.1M-Noncleavable BiTE

To reduce FcγR activation and Fc-mediated toxicity, hIgG Fc with mutations of LALA-G237A (L234A, L235A, and G237A) was generated to abolish FcγR and C1q interactions without affecting Fc stability. The full-length gene sequence of 1.6.1M proBiTE in which the human IgG1 monomeric Fc is engineered was synthesized by Biointron (Shanghai).

As a negative control, 1.6.1M-Unmask iBiTE was generated with primer pairs 1.6.1M-CTR-HC-FP/1.6.1M-CTR-HC-FP, in which the cleavable linker was excluded (FIG. 10), the mutant IgG1 Fc gene fragments were PCR amplified from the heavy chain of 1.6.1M as a template with primer pairs BI-Fc-3-FP/RP. For positive control, 1.6.1M-Noncleavable bispecific antibody was designed where the scFv of anti-EGFR antibody was fused via the (G₄S)4 linker to the N terminus of the VL-CL of anti-CD3 antibody. A mutant protease cleavage site was introduced to the N terminus of the VH domains of the Fab of a CD3 antibody (FIG. 10) with primer pairs E005-1.6.1M-NON-HC-FP/RP. The primers used are as following:

1.6.1M-CTR-HC-FP,
5′-GAGGTGCAGCTGGTGGAGAGCGGG-3′;
1.6.1M-CTR-HC-FP,
5′-GGCCCTCACCCCAGTCAGGAGGAC-3′;
BI-Fc-3-FP,
5′-GGGGCACCGTCAGTCTTCCTCT-3′;
BI-Fc-3-RP,
5′-AGCTGCTTCAGGTGCTGGGCACGGTGG-3′;
E005-1.6.1M-NON-HC-FP,
5′-GGAGCAGGGAACCACGGAGGAGGCGGAAGCGAGGTGC
AGCTG-3′;
E005-1.6.1M-NON-HC-RP,
5′-TCCGCTCAGTCCGCTTCCCCCTCCTCCGCTTCCTCCTCC
GAT-3′.

The full-length amino acid sequences of 1.6.1M proBiTE, 1.6.1M-Unmask iBiTE and 1.6.1M-Noncleavable BiTE are shown as follows.
1.6.1M proBiTE

Heavy chain
(SEQ ID NO: 66)

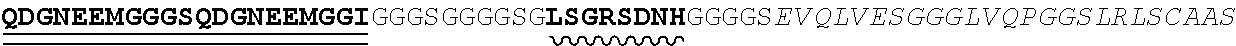

GFTFNTYAMNWVRQAPGKGLEWVARIRSKYNNYATYYADSVKGRFTISRDDSKNTLYLQMNSLRAEDTAVYYCAR
HGNFGSSYVSYFAYWGQGTTVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCPPCPAPE PSVFLFPPKPKDTL
MISRTPEVTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLLCLVKGFYPSDIAVEWESNGQPENNYKTTPPVLDSD
GSFFLHSKLTVDKSRWQQGNVFSCSVMHEALHNHYTQKSLSLSPGK,

wherein the mask peptide is shown as bold and double underline; the protease cleavage site is shown as bold and wavy line; anti-CD3 VH is shown as italics and HCDRs 1-3 therein is double underlined; CH1-CH2-CH3 is shown as italics and double underline and AAGA mutation (L234A, L235A, and G237A) therein is bolded.

Light chain
(SEQ ID NO: 67)
EVQLVETGGGVVQPGRSLRLSCAAS [symbol] MHWVRQAPGKGLEWVAV [symbol] YYADSVKG
RFTISRDNSKNTLYLQMNSLRAEDTAVYYC [symbol] WGQGTLVTVSSGGGGSGGGGSGGG
GSGGGGSDVVMTQSPSTLSASVGDRVTITCRAS [symbol] LAWYQQKPGKGPKLLIY [symbol] TLQSGVPSRFSGSGSGT
EFTLTISSLQPEDFATYYC [symbol] FGPGTKVDLKGGGGSGGGGSGGGGSGGGGSEIVVTQSPATLSVS
PGERATLSCRSS [symbol] ANWVQQKPGQAPRGLIG [symbol] KRAPGVPARFSGSLSGDEATLTISSLQSEDF
AVYYC [symbol] FGQGTKLEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGECPPCPAPEAAGAPSVFLFPP
KPKDTLMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLLCLVKGFYPSDIAVEWESNGQPENNYKTTP
PVLDSDGSFFLHSKLTVDKSRWQQGNVFSCSVMHEALHNHYTQKSLSLSPGK,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline and AAGA mutation (L234A, L235A, and G237A) therein is bolded.
1.6.1M-Unmask iBiTE

Heavy chain
(SEQ ID NO: 81)
EVQLVESGGGLVQPGGSLRLSCAAS [symbol] MNWVRQAPGKGLEWVAR [symbol] YYADSV
KGRFTISRDDSKNTLYLQMNSLRAEDTAVYYC [symbol] WGQGTTVTVSSASTKGPSVF
PLAPSSKSTSGGTAALGCLVKDYFPEPVTVSWNSGALTSGVHTFPAVLQSSGLYSLSSVVTVPSSSLGTQTYICNVNH
KPSNTKVDKKVEPKSCPPCPAPEAAGAPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLPPSRDELTK
NQVSLLCLVKGFYPSDIAVEWESNGQPENNYKTTPPVLDSDGSFFLHSKLTVDKSRWQQGNVFSCSVMHEALHN
HYTQKSLSLSPGK,

wherein anti-CD3 VH is shown as bold and HCDRs 1-3 therein is double underlined; CH1-CH2-CH3 is shown as italics and double underline and AAGA mutation (L234A, L235A, and G237A) therein is bolded.

Light chain
(SEQ ID NO: 67)
EVQLVETGGGVVQPGRSLRLSCAAS [symbol]
MHWVRQAPGKGLEWVAV [symbol]
YYADSVKGRFTISRDNS
KNTLYLQMNSLRAEDTAVYYC [symbol]
WGQGTLVTVSS
GGGGSGGGGSGGGGSGGGGSDVVM
TQSPSTLSASVGDRVTITCRAS [symbol]
LAWYQQKPGKGPKLLIY [symbol]
TLQSGVPSRFSGSGSGTEFTLTISS
LQPEDFATYYC [symbol]
FGPGTKVDLKGGGGSGGGGSGGGGSGGGGS
EIVVTQSPATLSVSPGERATLSCRSS [symbol]
ANWVQQKPGQAPRGLIG [symbol]
KRAPGVPARFSGSLSGDEATLTIS
SLQSEDFAVYYC [symbol]
FGQGTKLEIK
RTVAAPSVFIFPPSDEQLKSGTASVVCLL
NNFYPREAKVQWKVDNALQSGNSQESVTE
QDSKDSTYSLSSTLTLSKADYEKHKVYAC
EVTHQGLSSPVTKSFNRGECPPCPAPE
AAGAPSVFLFPPKPKDTLMISRTPEVTCVVV
DVSHEDPEVKFNWYVDGVEVHNAKTKPRE
EQYNSTYRVVSVLTVLHQDWLNGKEY

[symbol]

[symbol]

[symbol]

[symbol]

[symbol]

[symbol]

[symbol]

,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline and AAGA mutation (L234A, L235A, and G237A) therein is bolded.

### 1.6.1M-Noncleavable BiTE

Heavy chain
(SEQ ID NO: 82)

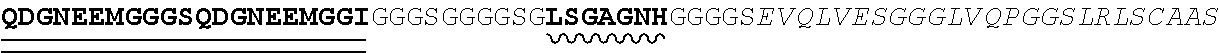

GFTENTYAMNWVRQAPGKGLEWVARIRSKYNNYATYYADSVKGRFTISRDDSKNTLYLQMNSLRAEDTAVYYCAR
HGNFGSSYVSYFAYWGQGTTVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCPPCPAPE [symbol] PSVFLFPPKPKDTL
MISRTPEVTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLLCLVKGFYPSDIAVEWESNGQPENNYKTTPPVLDSD
GSFFLHSKLTVDKSRWQQGNVFSCSVMHEALHNHYTQKSLSLSPGK,

wherein the mask peptide is shown as bold and double underline; the mutant protease cleavage site is shown as bold and wavy line; anti-CD3 VH is shown as italics and HCDRs 1-3 therein is double underlined; CH1-CH2-CH3 is shown as italics and double underline and AAGA mutation (L234A, L235A, and G237A) therein is bolded.

Light chain
(SEQ ID NO: 67)
EVQLVETGGGVVQPGRSLRLSCAAS [symbol]
MHWVRQAPGKGLEWVAV [symbol]
YYADSVKGRFTISRDNS
KNTLYLQMNSLRAEDTAVYYC [symbol]
WGQGTLVTVSS
GGGGSGGGGSGGGGSGGGGSDVV
MTQSPSTLSASVGDRVTITCRAS
QSMSRWLAWYQQKPGKGPKLLIY
ATSTLQSGVPSRFSGSGSGTEFTLT
ISSLQPEDFATYYCQQLDSFPFSFT
FGPGTKVDLKGGGGSGGGGSGGG
GSGGGGS
EIVVTQSPATLSVSPGERATLSCRSS

[symbol]

ANWVQQKPGQAPRGLIG [symbol]
KRAPGVPARFSGSLSGDEA
TLTISSLQSEDFAVYYC [symbol]
FGQGTKLEIK

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline and AAGA mutation (L234A, L235A, and G237A) therein is bolded.
3.2. Cloning of 1.6.1M-Pa/1.6.1M-2-1-Pa proBiTEs

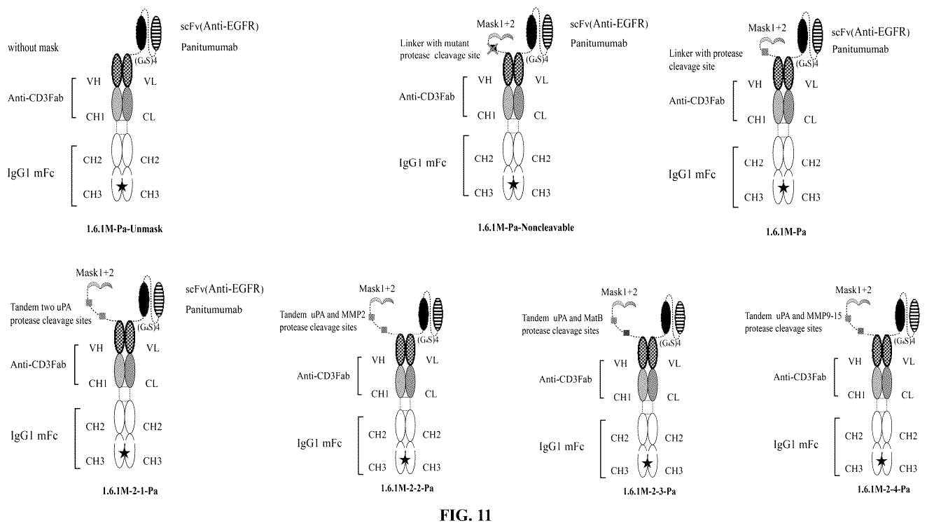

To improve the binding affinity to tumor antigen, 1.6.1M-Pa and 1.6.1M-2-1-Pa proBiTEs (FIG. 11) were designed. These proBiTEs were cloned by using 1.6.1M proBiTE as a template. The scFv of anti-EGFR antibody Panitumumab was fused via the (G₄S)₄ linker to the N terminus of the VL-CL with primer pairs E005-1.6.1M-2-1-Pa-FP/RP. The following primers were used:

E005-1.6.1M-2-1-Pa-FP,
5′-TGAGGGCCCAGGTGCAGCTGCAGG-3′;
E005-1.6.1M-2-1-Pa-R,
5′-GAACCACCTCCGCCACTTCCACCCCCTC
CTTTGATTTCCACCTTGGTTCCG-3′.

The full-length amino acid sequences of 1.6.1M-Pa proBiTE are shown as follows.

Heavy chain
(SEQ ID NO: 66)

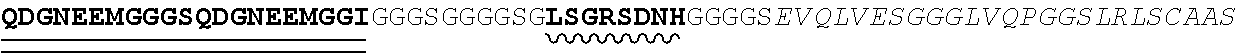

[symbol]

MNWVRQAPGKGLEWVAR [symbol] YYADSVKGRFTISRDDSKNTLYLQMNSLRAEDTAVYYC [symbol]
HGNFGSSYVSYFAYWGQGTTVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCPPCPAPE PSVELFPPKPKDTL
MISRTPEVTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELTKNQVSLLCLVKGFYPSDIAVEWESNGQPENNYKTTPPVLDSD [symbol] [symbol] [symbol]
,

wherein the mask peptide is shown as bold and double underline; the protease cleavage site is shown as bold and wavy line; anti-CD3 VH is shown as italics and HCDRs 1-3 therein is double underlined; CH1-CH2-CH3 is shown as italics and double underline and AAGA mutation (L234A, L235A, and G237A) therein is bolded.

Light chain
(SEQ ID NO: 68)
QVQLQESGPGLVKPSETLSLTCTVS [symbol]
WTWIRQSPGKGLEWI
GH [symbol] NYNPSLKSRLTISIDT
SKTQFSLKLSSVTAADTAIYYC [symbol]
WGQGTMVTVSS
GGGGSGGGGSGGGGSGGGG
SDIQMTQSPSSLSASVGDRVTITCQAS
QDISNYLNWYQQKPGKAPKLLIY [symbol]
NLETGVPSRFSGSGSGTDFTFTISSL
QPEDIATYFC [symbol] FGGGTKV
EIKGGGGSGGGGSGGGGSGGGGS
EIVVTQSPATLSVSPGERATLSCRSS [symbol]
ANWVQQKPGQAPRGLIG [symbol]
KRAPGVPARFSGSLSGDEATLTI
SSLQSEDFAVYYC [symbol]
FGQGTKLEIK
RTVAAPSVFIFPPSDEQLKSGTASVVC
LLNNFYPREAKVQWKVDNALQSGNSQE
SVTEQDSKDSTYSLSSTLTLSKADYEK
HKVYACEVTHQGLSSPVTKSFNRGECP
PCPAPEAAGAPSVFLFPPKPKDTLMIS
RTPEVTCVVVDVSHEDPEVKFNWYVDG
VEVHNAKTKPREEQYNSTYRVVSVLTV
LHQDWLNGKEYKCKVSNKALPAPIEKT
ISKAKGQPREPQVYTLPPSRDELTKNQ
VSLLCLVKGFYPSDIAVEWESNGQPEN
NYKTTPPVLDSDGSFFLHSKLTVDKSR
WQQGNVFSCSVMHEALHNHYTQKSLSL
SPGK,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline and AAGA mutation (L234A, L235A, and G237A) therein is bolded.
3.3. Cloning of 1.6.1M-2-1-Pa, 1.6.1M-2-2-Pa, 1.6.1M-2-3-Pa and 1.6.1M-2-4-Pa proBiTEs

To test whether alteration of the composition of the cleavable linker could affect the protease cleavage efficiency and other properties of the bispecific antibodies, four 1.6.1M-Pa proBiTE variants were generated. 1.6.1M-2-1-Pa proBiTE contains a cleavable polypeptide linker comprising two identical tandem LSGRSDNH (uPA protease cleavage site) sequences. The other three proBiTE variants, 1.6.1M-2-2-Pa, 1.6.1M-2-3-Pa and 1.6.1M-2-4-Pa proBiTEs, contain a cleavable polypeptide linker comprising tandem LSGRSDNH (uPA protease cleavage site) and PLGLAG (MMP2 protease cleavage site)/PMAKK (matriptase B (MatB) protease cleavage site)/PGPAGMKGLP (MMP9 protease cleavage site), respectively (FIG. 11). The following primers were used:

1.6.1M-VH-FP,
5′-GGCGGAAGCGAGGTGCAGCTGGTGGAGA-3′;
1.6.1M-2-1-RP,
5′-ATGATTATCTGAGCGACCACTCAGTCCTCCGT
GGTTGTCGCTTCTTC-3′;
1.6.1M-2-2-RP,
5′-GCCTGCCAGGCCCAGTGGTCCTCCGTGGTTGT
CGCTTCTTC-3′;
1.6.1M-2-3-RP,
5′-TTTCTTTGCCATTGGTCCTCCGTGGTTGTCGC
TTCTTC-3′;
1.6.1M-2-4-RP,
5′-AGGCAGGCCTTTCATGCCCGCTGGTCCTGGTC
CTCCGTGGTTGTCGCTTCTTC-3′.

The full-length amino acid sequences of 1.6.1M-2-1-Pa, 1.6.1M-2-2-Pa, 1.6.1M-2-3-Pa and 1.6.1M-2-4-Pa proBiTEs are shown as follows.
1.6.1M-2-1-Pa proBiTE

Heavy chain
(SEQ ID NO: 69)

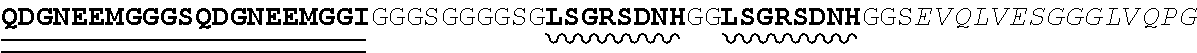

GSLRLSCAASGFTENTYAMNWVRQAPGKGLEWVARIRSKYNNYATYYADSVKGRFTISRDDSKNTLYLQMNSLRAE
DTAVYYCARHGNFGSSYVSYFAYWGQGTTVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKSCPPCPAPEAAGAPSVFL
FPPKPKDTLMISRTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLLCLVKGFYPSDIAVEWESNGQPENNYK
TTPPVLDSDGSFFLHSKLTVDKSRWQQGNVFSCSVMHEALHNHYTQKSLSLSPGK,

wherein the mask peptide is shown as bold and double underline; the protease cleavage sites are shown as bold and wavy line; anti-CD3 VH is shown as italics and HCDRs 1-3 therein is double underlined; CH1-CH2-CH3 is shown as italics and double underline and AAGA mutation (L234A, L235A, and G237A) therein is bolded.

Light chain
(SEQ ID NO: 68)
QVQLQESGPGLVKPSETLSLTCTVS [symbol]
WTWIRQSPGKGLEWI
GHI [symbol] NYNPSLKSRLTISIDT
SKTQFSLKLSSVTAADTAIYYC [symbol]
WGQGTMVTVSS
GGGGSGGGGSGGGGSGGGGS
DIQMTQSPSSLSASVGDRVTITCQAS
QDISNYLNWYQQKPGKAPKLLIYDAS
NLETGVPSRFSGSGSGTDFTFTISS
LQPEDIATYFCQHFDHLPLAFGGGT
KVEIKGGGGSGGGGSGGGGSGGGGS
EIVVTQSPATLSVSPGERATLSCRSS [symbol]
ANWVQQKPGQAPRGLIG [symbol]
KRAPGVPARFSGSLSGDEATLTI
SSLQSEDFAVYYC [symbol] F
GQGTKLEIK
RTVAAPSVFIFPPSDEQLKSGTASVVCL
LNNFYPREAKVQWKVDNALQSGNSQESV
TEQDSKDSTYSLSSTLTLSKADYEKHKV
YACEVTHQGLSSPVTKSFNRGECPPCPA
PEAAGAPSVFLFPPKPKDTLMISRTPEV
TCVVVDVSHEDPEVKFNWYVDGVEVHNA
KTKPREEQYNSTYRVVSVLTVLHQDWLN
GKEYKCKVSNKALPAPIEKTISKAKGQP
REPQVYTLPPSRDELTKNQVSLLCLVKG
FYPSDIAVEWESNGQPENNYKTTPPVLD
SDGSFFLHSKLTVDKSRWQQGNVFSCSV
MHEALHNHYTQKSLSLSPGK,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline and AAGA mutation (L234A, L235A, and G237A) therein is bolded.
1.6.1M-2-2-Pa proBiTE

Heavy chain
(SEQ ID NO: 70)

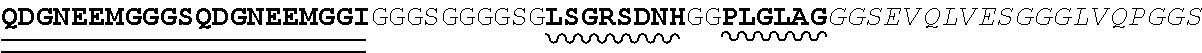

LRLSCAASGFTENTYAMNWVRQAPGKGLEWVARIRSKYNNYATYYADSVKGRFTISRDDSKNTLYLQMNSLRAEDT
AVYYCARHGNFGSSYVSYFAYWGQGTTVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKSCPPCPAPE [symbol] PSVFLFP
PKPKDTLMISRTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLLCLVKGFYPSDIAVEWESNGQPENNYKTT
PPVLDSDGSFFLHSKLTVDKSRWQQGNVFSCSVMHEALHNHYTQKSLSLSPGK,

wherein the mask peptide is shown as bold and double underline; the protease cleavage sites are shown as bold and wavy line; anti-CD3 VH is shown as italics and HCDRs 1-3 therein is double underlined; CH1-CH2-CH3 is shown as italics and double underline and AAGA mutation (L234A, L235A, and G237A) therein is bolded.

Light chain
(SEQ ID NO: 68)
QVQLQESGPGLVKPSETLSLTCTVS [symbol]
WTWIRQSPGKGLEWI
GH [symbol] NYNPSLKSRLTISIDT
SKTQFSLKLSSVTAADTAIYYC [symbol]
WGQGTMVTVSS
GGGGSGGGGSGGGGSGGGGS
DIQMTQSPSSLSASVGDRVTITCQAS
QDISNYLNWYQQKPGKAPKLLIY [symbol]
NLETGVPSRFSGSGSGTDFTFTISS
LQPEDIATYFC [symbol] FGGGT
KVEIKGGGGSGGGGSGGGGSGGGGS
EIVVTQSPATLSVSPGERATLSCRSS [symbol]
ANWVQQKPGQAPRGLIG [symbol]
KRAPGVPARFSGSLSGDEATLTI
SSLQSEDFAVYYC [symbol]
FGQGTKLEIK
RTVAAPSVFIFPPSDEQLKSGTASVVCL
LNNFYPREAKVQWKVDNALQSGNSQESV
TEQDSKDSTYSLSSTLTLSKADYEKHKV
YACEVTHQGLSSPVTKSFNRGECPPCPA
PEAAGAPSVFLFPPKPKDTLMISRTPEV
TCVVVDVSHEDPEVKFNWYVDGVEVHNA
KTKPREEQYNSTYRVVSVLTVLHQDWLN
GKEYKCKVSNKALPAPIEKTISKAKGQP
REPQVYTLPPSRDELTKNQVSLLCLVKG
FYPSDIAVEWESNGQPENNYKTTPPVLD
SDGSFFLHSKLTVDKSRWQQGNVFSCSV
MHEALHNHYTQKSLSLSPGK,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline and AAGA mutation (L234A, L235A, and G237A) therein is bolded.
1.6.1M-2-3-Pa proBiTE

Heavy chain
(SEQ ID NO: 71)

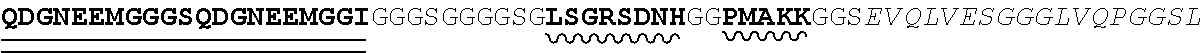

RLSCAASGFTENTYAMNWVRQAPGKGLEWVARIRSKYNNYATYYADSVKGRFTISRDDSKNTLYLQMNSLRAEDTA
VYYCARHGNFGSSYVSYFAYWGQGTTVTVSSASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEPKSCPPCPAPE [symbol] PSVFLFPP
KPKDTLMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLLCLVKGFYPSDIAVEWESNGQPENNYKTTP
PVLDSDGSFFLHSKLTVDKSRWQQGNVFSCSVMHEALHNHYTQKSLSLSPGK,

wherein the mask peptide is shown as bold and double underline; the protease cleavage sites are shown as bold and wavy line; anti-CD3 VH is shown as italics and HCDRs 1-3 therein is double underlined; CH1-CH2-CH3 is shown as italics and double underline and AAGA mutation (L234A, L235A, and G237A) therein is bolded.

Light chain
(SEQ ID NO: 68)
QVQLQESGPGLVKPSETLSLTCTVS [symbol]
WTWIRQSPGKGLEWI
GH [symbol] NYNPSLKSRLTISIDT
SKTQFSLKLSSVTAADTAIYYC [symbol]
WGQGTMVTVSS
GGGGSGGGGSGGGGSGGGGS
DIQMTQSPSSLSASVGDRVTITCQAS [symbol]
LNWYQQKPGKAPKLLIY [symbol]
NLETGVPSRFSGSGSGTDFTFTISS
LQPEDIATYFC [symbol] FGGGT
KVEIKGGGGSGGGGSGGGGSGGGGS
EIVVTQSPATLSVSPGERATLSCRSS [symbol]
ANWVQQKPGQAPRGLIG [symbol]
KRAPGVPARFSGSLSGDEATLTI
SSLQSEDFAVYYC [symbol]
FGQGTKLEIK
RTVAAPSVFIFPPSDEQLKSGTASVVCL
LNNFYPREAKVQWKVDNALQSGNSQESV
TEQDSKDSTYSLSSTLTLSKADYEKHKV
YACEVTHQGLSSPVTKSFNRGECPPCPA
PEAAGAPSVELFPPKPKDTLMISRTPEV
TCVVVDVSHEDPEVKFNWYVDGVEVHNA
KTKPREEQYNSTYRVVSVLTVLHQDWLN
GKEYKCKVSNKALPAPIEKTISKAKGQP
REPQVYTLPPSRDELTKNQVSLLCLVKG
FYPSDIAVEWESNGQPENNYKTTPPVLD
SDGSFFLHSKLTVDKSRWQQGNVFSCSV
MHEALHNHYTQKSLSLSPGK,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline and AAGA mutation (L234A, L235A, and G237A) therein is bolded.
1.6.1M-2-4-Pa proBiTE

Heavy chain
(SEQ ID NO: 72)

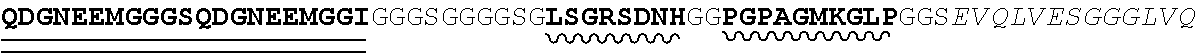

PGGSLRLSCAAS [symbol] MNWVRQAPGKGLEWVAR [symbol] YYADSVKGRFTISRDDSKNTLYLQMNSLR
AEDTAVYYCARHGNFGSSYVSYFAYWGQGTTVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKKVEPKSCPPCPAPE [symbol] PSV
FLFPPKPKDTLMISRTPEVTCVVVDVSHEDPEVKENWYVDGVEVHNAKTKPREEQYNSTYRVVSVLTVLHQDWLN
GKEYKCKVSNKALPAPIEKTISKAKGQPREPQVYTLPPSRDELTKNQVSLLCLVKGFYPSDIAVEWESNGQPENNY [symbol] [symbol] [symbol]
,

wherein the mask peptide is shown as bold and double underline; the protease cleavage sites are shown as bold and wavy line; anti-CD3 VH is shown as italics and HCDRs 1-3 therein is double underlined; CH1-CH2-CH3 is shown as italics and double underline and AAGA mutation (L234A, L235A, and G237A) therein is bolded.

Light chain
(SEQ ID NO: 68)
QVQLQESGPGLVKPSETLSLTCTVS [symbol]
WTWIRQSPGKGLEWI
GH [symbol] NYNPSLKSRLTISIDT
SKTQFSLKLSSVTAADTAIYYC [symbol]
WGQGTMVTVSS
GGGGSGGGGSGGGGSGGGGS
DIQMTQSPSSLSASVGDRVTITCQAS [symbol]
LNWYQQKPGKAPKLLIY [symbol]
NLETGVPSRFSGSGSGTDFTFTISS
LQPEDIATYFC [symbol] FGGGT
KVEIKGGGGSGGGGSGGGGSGGGGS
EIVVTQSPATLSVSPGERATLSCRSS [symbol]
ANWVQQKPGQAPRGLIG
GANKRAPGVPARFSGSLSGDEATLTI
SSLQSEDFAVYYC [symbol]
FGQGTKLEIK
RTVAAPSVFIFPPSDEQLKSGTASVVCL
LNNFYPREAKVQWKVDNALQSGNSQESV
TEQDSKDSTYSLSSTLTLSKADYEKHKV
YACEVTHQGLSSPVTKSFNRGECPPCPA
PEAAGAPSVFLFPPKPKDTLMISRTPEV
TCVVVDVSHEDPEVKFNWYVDGVEVHNA
KTKPREEQYNSTYRVVSVLTVLHQDWLN
GKEYKCKVSNKALPAPIEKTISKAKGQP
REPQVYTLPPSRDELTKNQVSLLCLVKG
FYPSDIAVEWESNGQPENNYKTTPPVLD
SDGSFFLHSKLTVDKSRWQQGNVFSCSV
MHEALHNHYTQKSLSLSPGK,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline and AAGA mutation (L234A, L235A, and G237A) therein is bolded.
3.4. Cloning of 1.6.1M-Pa-Unmask iBiTE and 1.6.1M-Pa-Noncleavabe BITE

As control of 1.6.1M-Pa proBiTE, 1.6.1M-Pa-Unmask iBiTE and 1.6.1M-Pa-Noncleavabe BiTE were generated with the same primers as 1.6.1M-Unmask iBITE (FIG. 11).

The full-length amino acid sequences of 1.6.1M-Pa-Unmask iBiTE and 1.6.1M-Pa-Noncleavabe BiTE are shown as follows.

1.6.1M-Pa-Unmask iBiTE

Heavy chain
(SEQ ID NO: 83)
EVQLVESGGGLVQPGGSLRLSCAASGFTFNTYAMNWVRQAPGKGLEW
VARIRSKYNNYATYYADSVKGRFTISRDDSKNTLYLQMNSLRAEDTA
VYYCARHGNFGSSYVSYFAYWGQGTTVTVSSASTKGPSVFPLAPSSK
STSGGTAALGCLVKDYFPEPVTVSWNSGALTSGVHTFPAVLQSSGLY
SLSSVVTVPSSSLGTQTYICNVNHKPSNTKVDKKVEPKSCPPCPAPE
AAGAPSVFLFPPKPKDTLMISRTPEVTCVVVDVSHEDPEVKFNWYVD
GVEVHNAKTKPREEQYNSTYRVVSVLTVLHQDWLNGKEYKCKVSNKA
LPAPIEKTISKAKGQPREPQVYTLPPSRDELTKNQVSLLCLVKGFYP
SDIAVEWESNGQPENNYKTTPPVLDSDGSFFLHSKLTVDKSRWQQGN
VFSCSVMHEALHNHYTQKSLSLSPGK,

wherein anti-CD3 VH is shown as bold and HCDRs 1-3 therein is double underlined; CH1-CH2-CH3 is shown as italics and AAGA mutation (L234A, L235A, and G237A) therein is bolded.

Light chain
(SEQ ID NO: 68)
QVQLQESGPGLVKPSETLSLTCTVSGGSVSSGDYYWTWIRQSPGKGL
EWIGHIYYSGNTNYNPSLKSRLTISIDTSKTQFSLKLSSVTAADTAI
YYCVRDRVTGAFDIWGQGTMVTVSSGGGGSGGGGSGGGGSGGGGSDI
QMTQSPSSLSASVGDRVTITCQAS [symbol] LNWYQQKPGKAPKLLIY
DASNLETGVPSRFSGSGSGTDFTFTISSLQPEDIATYFCQHFDHLPL
AFGGGTKVEIKGGGGSGGGGSGGGGSGGGGSEIVVTQSPATLSVSPG
ERATLSCRSSTGAVTTSNYANWVQQKPGQAPRGLIGGANKRAPGVPA
RFSGSLSGDEATLTISSLQSEDFAVYYCALWYSNLWVFGQGTKLEIK
RTVAAPSVFIFPPSDEQLKSGTASVVCLLNNFYPREAKVQWKVDNAL
QSGNSQESVTEQDSKDSTYSLSSTLTLSKADYEKHKVYACEVTHQGL
SSPVTKSENRGECPPCPAPEAAGAPSVELFPPKPKDTLMISRTPEVT
CVVVDVSHEDPEVKENWYVDGVEVHNAKTKPREEQYNSTYRVVSVLT
VLHQDWLNGKEYKCKVSNKALPAPIEKTISKAKGQPREPQVYTLPPS
RDELTKNQVSLLCLVKGFYPSDIAVEWESNGQPENNYKTTPPVLDSD
GSFFLHSKLTVDKSRWQQGNVESCSVMHEALHNHYTQKSLSLSPGK,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline and AAGA mutation (L234A, L235A, and G237A) therein is bolded.

### 1.6.1M-Pa-Noncleavabe BiTE

Heavy chain
(SEQ ID NO: 84)

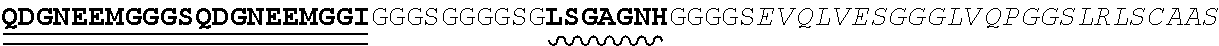

[symbol]

MNWVRQAPGKGLEWVAR [symbol] YYADSVKGRFTISRDDSKNTLYLQMNSLRAEDTAVYYC [symbol] [symbol]
WGQGTTVTVSS

[symbol]

[symbol]

[symbol]

[symbol]

[symbol]

[symbol]

[symbol]

MISRTPEVTCVVVDVSHEDPEVKENWYVDGVEVHNAKTKPREEQYNSTYRVVSVLTVLHQDWLNGKEYKCKVSN
KALPAPIEKTISKAKGQPREPQVYTLPPSRDELTKNQVSLLCLVKGFYPSDIAVEWESNGQPENNYKTTPPVLDSD
GSFFLHSKLTVDKSRWQQGNVFSCSVMHEALHNHYTQKSLSLSPGK,

wherein the mask peptide is shown as bold and double underline; the mutant protease cleavage site is shown as bold and wavy line; anti-CD3 VH is shown as italics and HCDRs 1-3 therein is double underlined; CH1-CH2-CH3 is shown as italics and double underline and AAGA mutation (L234A, L235A, and G237A) therein is bolded.

Light chain
(SEQ ID NO: 68)
QVQLQESGPGLVKPSETLSLTCTVSGGSVSSGDYYWTWIRQSPGKGL
EWIGHIYYSGNTNYNPSLKSRLTISIDTSKTQFSLKLSSVTAADTAI
YYCVRDRVTGAFDIWGQGTMVTVSSGGGGSGGGGSGGGGSGGGGSDI
QMTQSPSSLSASVGDRVTITCQAS [symbol] LNWYQQKPGKAPKLLIY [symbol]
NLETGVPSRFSGSGSGTDFTFTISSLQPEDIATYFC [symbol]
FGGGTKVEIKGGGGSGGGGSGGGGSGGGGSEIVVTQSPATLSVSPG
ERATLSCRSS [symbol] ANWVQQKPGQAPRGLIG [symbol] KRAPGVPA
RFSGSLSGDEATLTISSLQSEDFAVYYC [symbol] FGQGTKLEIK
RTVAAPSVFIFPPSDEQLKSGTASVVCLLNNFYPREAKVQWKVDNAL
QSGNSQESVTEQDSKDSTYSLSSTLTLSKADYEKHKVYACEVTHQGL
SSPVTKSFNRGECPPCPAPEAAGAPSVFLFPPKPKDTLMISRTPEVT
CVVVDVSHEDPEVKFNWYVDGVEVHNAKTKPREEQYNSTYRVVSVLT
VLHQDWLNGKEYKCKVSNKALPAPIEKTISKAKGQPREPQVYTLPPS
RDELTKNQVSLLCLVKGFYPSDIAVEWESNGQPENNYKTTPPVLDSD
GSFFLHSKLTVDKSRWQQGNVFSCSVMHEALHNHYTQKSLSLSPGK,

wherein anti-EGFR VH is shown as bold and HCDRs 1-3 therein is double underlined; anti-EGFR VL is shown as italics and LCDRs 1-3 therein is double underlined; anti-CD3 VL is shown as bold and italics and LCDRs 1-3 therein is double underlined; and CL-CH2-CH3 is shown as italics and double underline and AAGA mutation (L234A, L235A, and G237A) therein is bolded.
3.5. Cloning of 1.4.3-Pa, 1.4.3M and 1.4.3M-Pa proB iTEs

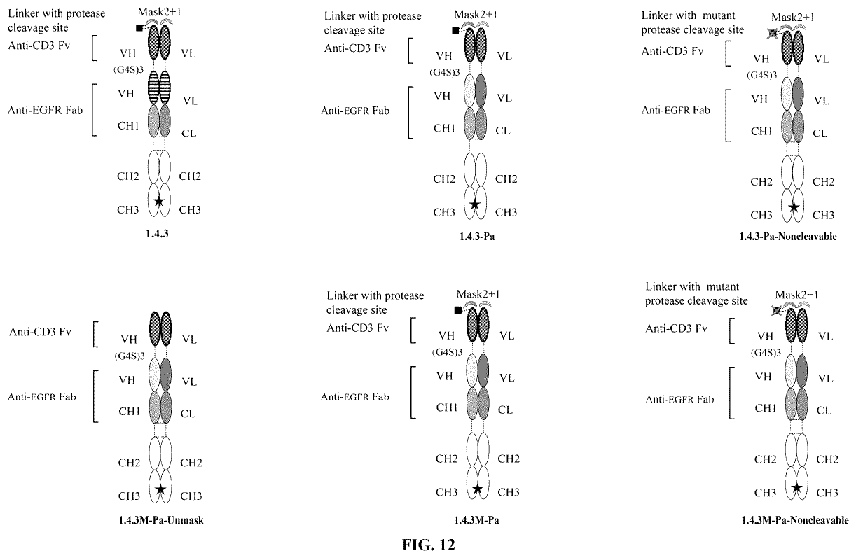

To compare the difference between the formats of 1.4.3 and 1.6.1 proBiTEs, 1.4.3-Pa, 1.4.3M and 1.4.3M-Pa proBiTEs were generated. 1.4.3-Pa proBiTE includes the same domains of 1.4.3 proBiTE except that the anti-EGFR Fv was replaced with anti-Panitumumab Fv, 1.4.3-M proBiTE includes the same domains of 1.4.3 proBiTE except that Fc regions in both heavy chain and light chain contains AAGA mutation (L234A, L235A, and G237A), and 1.4.3M-Pa proBiTE includes the same domains of 1.4.3 proBiTE except that the anti-EGFR Fv was replaced with anti-Panitumumab Fv and Fc regions in both heavy chain and light chain contains AAGA mutation (L234A, L235A, and G237A) (FIG. 12). The following primers were used:

1.4.3-Pa-VH-FP,
5′-AGGCGGTAGTCAGGTGCAGCTGCAGGAG-3′;
1.4.3-Pa-VH-RP,
5′-GCTGCTCACGGTCACCAT-3′;
1.4.3-Pa-VL-FP,
5′-GCGGTAGTGACATTCAGATGACCCAGA-3′;
1.4.3-Pa-VL-RP,
5′-GCCACCGTACGTTTGATCTCCACCTTGGTG-3′;

The full-length amino acid sequences of 1.4.3-Pa and 1.4.3M-Pa proBiTEs are shown as follows.
1.4.3-Pa proBiTE

Heavy chain
(SEQ ID NO: 77)

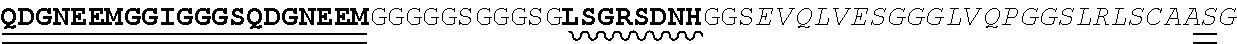

FTFNTYAMNWVRQAPGKGLEWVARIRSKYNNYATYYADSVKGRFTISRDDSKNTLYLQMNSLRAEDTAVYYCARH
GNFGSSYVSYFAYWGQGTTVTVSSGGGSSGGGGSGGGGSQVQLQESGPGLVKPSETLSLTCTVSGGSVSSG
DYYWTWIRQSPGKGLEWIGHIYYSGNTNYNPSLKSRLTISIDTSKTQFSLKLSSVTAADTAIYYCVRDR
VTGAFDIWGQGTMVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCPPCPAPELLGGPSVFLFPPKPKDTLMISRT
PEVTCVVVDVSHEDPEVKFNWYVDGVEVHNAKTKPREEQYNSTYRVVSVLTVLHQDWLNGKEYKCKVSNKALPA
PIEKTISKAKGQPREPQVYTLPPSRDELTKNQVSLLCLVKGFYPSDIAVEWESNGQPENNYKTTPPVLDSDGSFFL
HSKLTVDKSRWQQGNVFSCSVMHEALHNHYTQKSLSLSPGK,

wherein the mask peptide is shown as bold and double underline; the protease cleavage site is shown as bold and wavy line; anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline.

Light chain
(SEQ ID NO: 78)
EIVVTQSPATLSVSPGERATLSCRSS [symbol] ANWVQQKPG
QAPRGLIG [symbol] KRAPGVPARFSGSLSGDEATLTISSLQSEDFAV
YYC [symbol] FGQGTKLEIKGGGGSGGGGSGGGGSDIQMTQS
PSSLSASVGDRVTITCQAS [symbol] LNWYQQKPGKAPKLLIY [symbol]
NLETGVPSRFSGSGSGTDFTFTISSLQPEDIATYFC [symbol] [symbol]
FGGGTKVEIKRTVAAPSVFIFPPSDEQLKSGTASVVCLLNNF
YPREAKVQWKVDNALQSGNSQESVTEQDSKDSTYSLSSTLTLSK
ADYEKHKVYACEVTHQGLSSPVTKSFNRGECPPCPAPELLGGPS
VFLFPPKPKDTLMISRTPEVTCVVVDVSHEDPEVKFNWYVDGVE
VHNAKTKPREEQYNSTYRVVSVLTVLHQDWLNGKEYKCKVSNKA
LPAPIEKTISKAKGQPREPQVYTLPPSRDELTKNQVSLLCLVKG
FYPSDIAVEWESNGQPENNYKTTPPVLDSDGSFFLHSKLTVDKS
RWQQGNVFSCSVMHEALHNHYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline.
1.4.3M-Pa proBiTE

Heavy chain
(SEQ ID NO: 73)

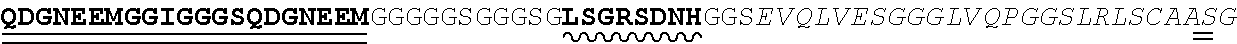

FTFNTYAMNWVRQAPGKGLEWVARIRSKYNNYATYYADSVKGRFTISRDDSKNTLYLQMNSLRAEDTAVYYCARH
GNFGSSYVSYFAYWGQGTTVTVSSGGGSSGGGGSGGGGSQVQLQESGPGLVKPSETLSLTCTVSGGSVSSG
DYYWTWIRQSPGKGLEWIGHIYYSGNTNYNPSLKSRLTISIDTSKTQFSLKLSSVTAADTAIYYCVRDR
VTGAFDIWGQGTMVTVSSASTKGPSVFPLAPSSKSTSGGTAALGCLVKDYFPEPVTVSWNSGALTSGVHTEPAV
LQSSGLYSLSSVVTVPSSSLGTQTYICNVNHKPSNTKVDKKVEPKSCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKVSNKALPA
PIEKTISKAKGQPREPQVYTLPPSRDELTKNQVSLLCLVKGFYPSDIAVEWESNGQPENNYKTTPPVLDSDGSFFL
HSKLTVDKSRWQQGNVESCSVMHEALHNHYTQKSLSLSPGK,

wherein the mask peptide is shown as bold and double underline; the protease cleavage site is shown as bold and wavy line; anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline and LALA mutation (L234A and L235A) therein is bolded.

Light chain
(SEQ ID NO: 74)
EIVVTQSPATLSVSPGERATLSCRSS [symbol] ANWVQQKPG
QAPRGLIG [symbol] KRAPGVPARFSGSLSGDEATLTISSLQSEDFAV
YYC [symbol] FGQGTKLEIKGGGGSGGGGSGGGGSDIQMTQS
PSSLSASVGDRVTITCQAS [symbol] LNWYQQKPGKAPKLLIY [symbol]
NLETGVPSRFSGSGSGTDFTFTISSLQPEDIATYFC [symbol] [symbol]
FGGGTKVEIKRTVAAPSVFIFPPSDEQLKSGTASVVCLLNNF
YPREAKVQWKVDNALQSGNSQESVTEQDSKDSTYSLSSTLTLSK
ADYEKHKVYACEVTHQGLSSPVTKSFNRGECPPCPAPEAAGGPS
VFLFPPKPKDTLMISRTPEVTCVVVDVSHEDPEVKFNWYVDGVE
VHNAKTKPREEQYNSTYRVVSVLTVLHQDWLNGKEYKCKVSNKA
LPAPIEKTISKAKGQPREPQVYTLPPSRDELTKNQVSLLCLVKG
FYPSDIAVEWESNGQPENNYKTTPPVLDSDGSFFLHSKLTVDKS
RWQQGNVFSCSVMHEALHNHYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline and LALA mutation (L234A and L235A) therein is bolded.

### 3.6. Cloning of 1.4.3-Pa-Noncleavabe, 1.4.3M-Pa-Unmask and 1.4.3M-Pa-Noncleavabe BIT Es

As control, 1.4.3-Pa-Noncleavabe, 1.4.3M-Pa-Unmask and 1.4.3M-Pa-Noncleavabe BiTEs (FIG. 12) were generated with the similar method as that in Example 3.5.

The full-length amino acid sequences of 1.4.3-Pa-Noncleavabe, 1.4.3M-Pa-Unmask and 1.4.3M-Pa-Noncleavabe BiTEs are shown as follows.

### 1.4.3-Pa-Noncleavabe BITE

Heavy chain
(SEQ ID NO: 85) [symbol]
GGGGGSGGGSGGGSEVQLVESGG
GLVQPGGSLRLSCAAS [symbol] MNWVRQAPGKGLEWVAR [symbol]
YYADSVKGRFTISRDDSKNTLYLQMNSLRAEDTAV
YYC [symbol] [symbol] WGQGTTVTVSSGGGSSGGGGSGGGG
SQVQLQESGPGLVKPSETLSLTCTVS [symbol] WTWIRQSPGK
GLEWIGH [symbol] NYNPSLKSRLTISIDTSKTQFSLKLSSVTA
ADTAIYYC [symbol] WGQGTMVTVSSASTKGPSVFPLAPS
SKSTSGGTAALGCLVKDYFPEPVTVSWNSGALTSGVHTFPAVLQ
SSGLYSLSSVVTVPSSSLGTQTYICNVNHKPSNTKVDKKVEPKS
CPPCPAPELLGGPSVFLFPPKPKDTLMISRTPEVTCVVVDVSHE
DPEVKFNWYVDGVEVHNAKTKPREEQYNSTYRVVSVLTVLHQDW
LNGKEYKCKVSNKALPAPIEKTISKAKGQPREPQVYTLPPSRDE
LTKNQVSLLCLVKGFYPSDIAVEWESNGQPENNYKTTPPVLDSD
GSFFLHSKLTVDKSRWQQGNVFSCSVMHEALHNHYTQKSLSLSP
GK,

wherein the mask peptide is shown as bold and double underline; anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline.

Light chain
(SEQ ID NO: 78)
EIVVTQSPATLSVSPGERATLSCRSS [symbol] ANWVQQKPG
QAPRGLIG [symbol] KRAPGVPARFSGSLSGDEATLTISSLQSEDFAV
YYC [symbol] FGQGTKLEIKGGGGSGGGGSGGGGSDIQMTQS
PSSLSASVGDRVTITCQAS [symbol] LNWYQQKPGKAPKLLIY [symbol]
NLETGVPSRFSGSGSGTDFTFTISSLQPEDIATYFC [symbol] [symbol]
FGGGTKVEIKRTVAAPSVFIFPPSDEQLKSGTASVVCLLNNF
YPREAKVQWKVDNALQSGNSQESVTEQDSKDSTYSLSSTLTLSK
ADYEKHKVYACEVTHQGLSSPVTKSFNRGECPPCPAPELLGGPS
VFLFPPKPKDTLMISRTPEVTCVVVDVSHEDPEVKFNWYVDGVE
VHNAKTKPREEQYNSTYRVVSVLTVLHQDWLNGKEYKCKVSNKA
LPAPIEKTISKAKGQPREPQVYTLPPSRDELTKNQVSLLCLVKG
FYPSDIAVEWESNGQPENNYKTTPPVLDSDGSFFLHSKLTVDKS
RWQQGNVFSCSVMHEALHNHYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline.

### 1.4.3M-Pa-Unmask BiTE

Heavy chain
(SEQ ID NO: 86)
EVQLVESGGGLVQPGGSLRLSCAAS [symbol] MNWVRQAPGK
GLEWVAR [symbol] YYADSVKGRFTISRDDSKNTLYLQMN
SLRAEDTAVYYC [symbol] WGQGTTVTVSSGGGS
SGGGGSGGGGSQVQLQESGPGLVKPSETLSLTCTVS [symbol]
WTWIRQSPGKGLEWIGH [symbol] NYNPSLKSRLTISIDT
SKTQFSLKLSSVTAADTAIYYC [symbol] WGQGTMVTVS
SASTKGPSVFPLAPSSKSTSGGTAALGCLVKDYFPEPVTVSWN
SGALTSGVHTFPAVLQSSGLYSLSSVVTVPSSSLGTQTYICNV
NHKPSNTKVDKKVEPKSCPPCPAPEAAGGPSVFLFPPKPKDTL
MISRTPEVTCVVVDVSHEDPEVKFNWYVDGVEVHNAKTKPREE
QYNSTYRVVSVLTVLHQDWLNGKEYKCKVSNKALPAPIEKTIS
KAKGQPREPQVYTLPPSRDELTKNQVSLLCLVKGFYPSDIAVE
WESNGQPENNYKTTPPVLDSDGSFFLHSKLTVDKSRWQQGNVF
SCSVMHEALHNHYTQKSLSLSPGK,

wherein anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline and double underline and LALA mutation (L234A and L235A) therein is bolded.

Light chain
(SEQ ID NO: 74)
EIVVTQSPATLSVSPGERATLSCRSS [symbol] ANWVQQKPG
QAPRGLIG [symbol] KRAPGVPARFSGSLSGDEATLTISSLQSEDFAV
YYC [symbol] FGQGTKLEIKGGGGSGGGGSGGGGSDIQMTQS
PSSLSASVGDRVTITCQAS [symbol] LNWYQQKPGKAPKLLIY [symbol]
NLETGVPSRFSGSGSGTDFTFTISSLQPEDIATYFC [symbol] [symbol]
FGGGTKVEIKRTVAAPSVFIFPPSDEQLKSGTASVVCLLNNF
YPREAKVQWKVDNALQSGNSQESVTEQDSKDSTYSLSSTLTLSK
ADYEKHKVYACEVTHQGLSSPVTKSFNRGECPPCPAPEAAGGPS
VFLFPPKPKDTLMISRTPEVTCVVVDVSHEDPEVKFNWYVDGVE
VHNAKTKPREEQYNSTYRVVSVLTVLHQDWLNGKEYKCKVSNKA
LPAPIEKTISKAKGQPREPQVYTLPPSRDELTKNQVSLLCLVKG
FYPSDIAVEWESNGQPENNYKTTPPVLDSDGSFFLHSKLTVDKS
RWQQGNVFSCSVMHEALHNHYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline and LALA mutation (L234A and L235A) therein is bolded.

### 1.4.3M-Pa-Noncleavabe BiTE

Heavy chain
(SEQ ID NO: 87) [symbol]
GGGGGSGGGSGGSEVQLVESG
GGLVQPGGSLRLSCAAS [symbol] MNWVRQAPGKGLEWVAR [symbol]
YYADSVKGRFTISRDDSKNTLYLQMNSLRAEDTA
VYYC [symbol] WGQGTTVTVSSGGGSSGGGGSGG
GGSQVQLQESGPGLVKPSETLSLTCTVS [symbol] WTWIR
QSPGKGLEWIGH [symbol] NYNPSLKSRLTISIDTSKTQFSLK
LSSVTAADTAIYYC [symbol] WGQGTMVTVSSASTKGPS
VFPLAPSSKSTSGGTAALGCLVKDYFPEPVTVSWNSGALTSGV
HTFPAVLQSSGLYSLSSVVTVPSSSLGTQTYICNVNHKPSNTK
VDKKVEPKSCPPCPAPEAAGGPSVFLFPPKPKDTLMISRTPEV
TCVVVDVSHEDPEVKFNWYVDGVEVHNAKTKPREEQYNSTYRV
VSVLTVLHQDWLNGKEYKCKVSNKALPAPIEKTISKAKGQPRE
PQVYTLPPSRDELTKNQVSLLCLVKGFYPSDIAVEWESNGQPE
NNYKTTPPVLDSDGSFFLHSKLTVDKSRWQQGNVFSCSVMHEA
LHNHYTQKSLSLSPGK,

wherein the mask peptide is shown as bold and double underline; anti-CD3 VH is shown as italics and HCDRs 1-3 therein is double underlined; anti-EGFR VH is shown as bold and HCDRs 1-3 therein is double underlined; CH1-CH2-CH3 is shown as italics and double underline and LALA mutation (L234A and L235A) therein is bolded.

Light chain
(SEQ ID NO: 74)
EIVVTQSPATLSVSPGERATLSCRSS [symbol] ANWVQQK
PGQAPRGLIG [symbol] KRAPGVPARFSGSLSGDEATLTISSLQSE
DFAVYYC [symbol] FGQGTKLEIKGGGGSGGGGSGGGGSD
IQMTQSPSSLSASVGDRVTITCQAS [symbol] LNWYQQKPGKA
PKLLIY [symbol] NLETGVPSRFSGSGSGTDFTFTISSLQPEDIAT
YFC [symbol] [symbol] FGGGTKVEIKRTVAAPSVFIFPPSDEQLKS
GTASVVCLLNNFYPREAKVQWKVDNALQSGNSQESVTEQDSK
DSTYSLSSTLTLSKADYEKHKVYACEVTHQGLSSPVTKSFNR
GECPPCPAPEAAGGPSVFLFPPKPKDTLMISRTPEVTCVVVD
VSHEDPEVKFNWYVDGVEVHNAKTKPREEQYNSTYRVVSVLT
VLHQDWLNGKEYKCKVSNKALPAPIEKTISKAKGQPREPQVY
TLPPSRDELTKNQVSLLCLVKGFYPSDIAVEWESNGQPENNY
KTTPPVLDSDGSFFLHSKLTVDKSRWQQGNVFSCSVMHEALH
NHYTQKSLSLSPGK,

wherein anti-CD3 VL is shown as italics and LCDRs 1-3 therein is double underlined; anti-EGFR VL is shown as bold and LCDRs 1-3 therein is double underlined; and CL-CH2-CH3 is shown as italics and double underline and LALA mutation (L234A and L235A) therein is bolded.

### 3.7. Protein Expression and Purification

All bispecific antibodies were expressed in CHO-S cells as described in Example 1 and purified from the CHO-S culture supernatant using Protein A Sepharose 4 Fast Flow column chromatography (GE Healthcare) according to the manufacturer's instructions.

The results show that bispecific antibodies were well expressed in transiently transfected CHO-S cells and secreted into the culture supernatants. On a non-reducing SDS-PAGE, CD3×EGFR proBiTE displays an apparent molecular weight (aMW) of approximately 150 kDa. On a reducing SDS-PAGE, the heavy chain and light chain have apparent molecular weight of approximately 75 kDa and 65 kDa, respectively (FIG. 6C).

### Example 4. Blocking Effect on Human CD3 and Binding Affinity to Human EGFR of Variant proBiTEs

To determine the binding affinity of the variant proBiTEs to recombinant human CD3 and EGFR, ELISA was performed as described in Example 2.

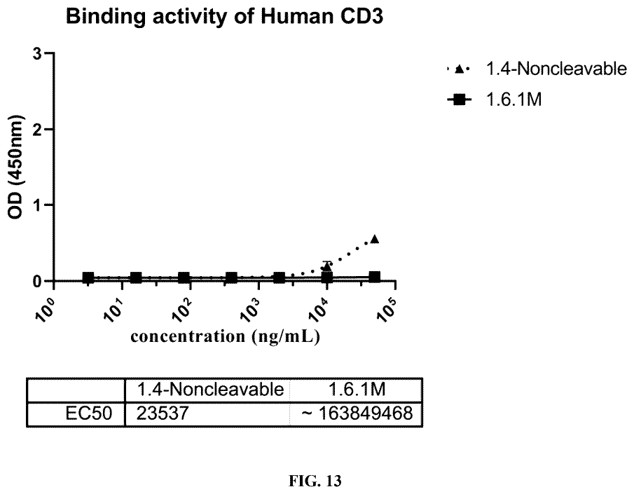

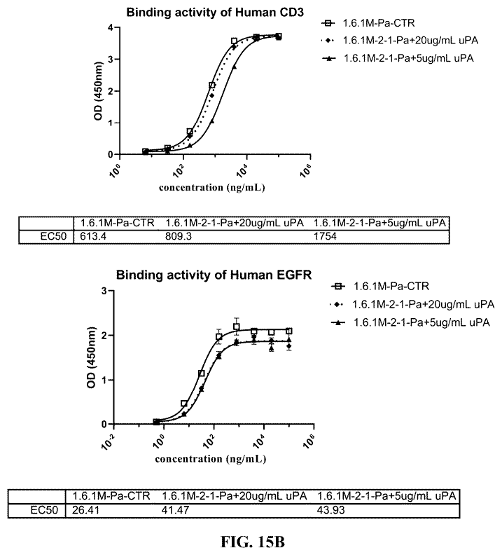

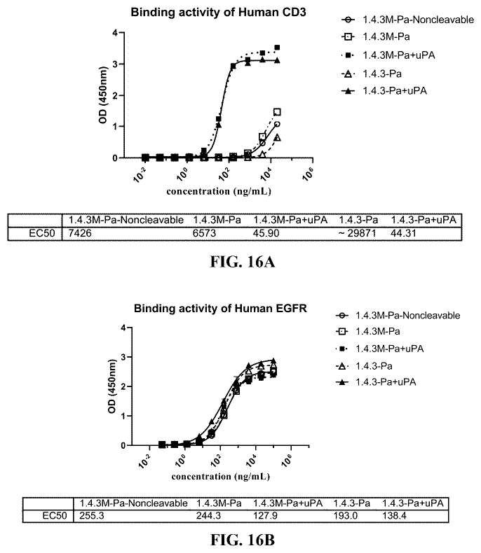

FIGS. 13-16 show that variant proBiTEs of the invention, particularly 1.6.1M-Pa, 1.6.1M-2-1-Pa and 1.4.3M-Pa proBiTEs, have strong blocking effect on human CD3 antigen, and the binding affinity of these proBiTEs against CD3 is recovered after protease digestion.

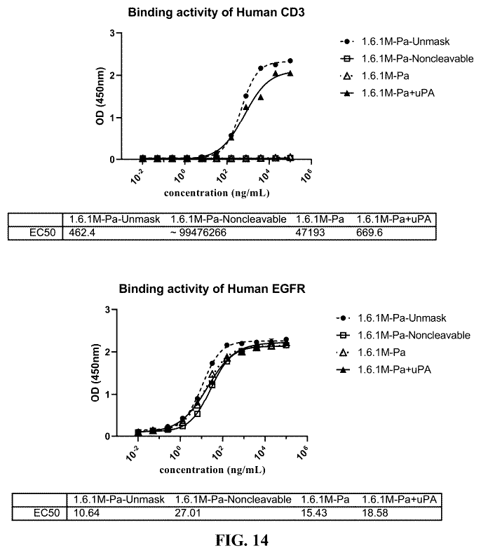

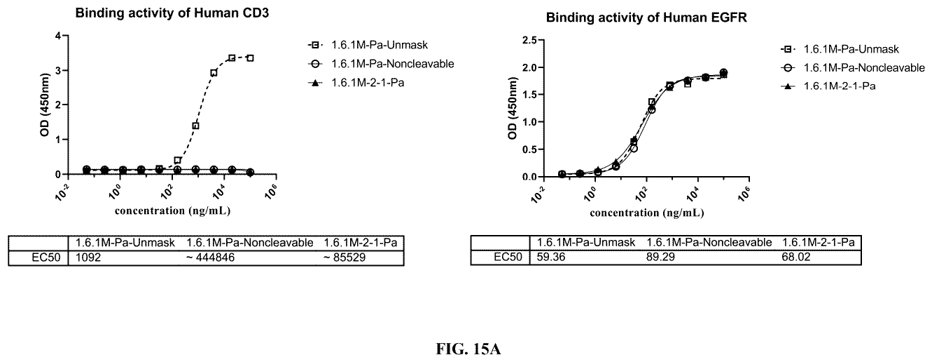

The results in FIGS. 14-16 also indicates that there was no significant difference in the binding activity of these variant proBiTEs to EGFR before and after uPA digestion.

### Example 5. Detection of Human uPA Content in Tumor Cell Culture Supernatant

To select tumor cell lines with high uPA expression, samples were measured by ELISA. The content of uPA in tumor cell culture supernatant was quantified by human Urokinase ELISA Pair Set. Monoclonal antibodies specific for human uPA were coated on a 96-well plate. Standards and samples were added to the wells. The wells were washed and a horseradish peroxidase conjugated mouse anti-human uPA monoclonal antibody was then added. The wells were again washed and TMB substrate solution was added, which develops color in proportion to the amount of human uPA present in the sample. To end the enzyme reaction, the stop solution was added and absorbance at 450 nm was read. The mean absorbance for each set of duplicate standards, controls and samples was calculated by subtracting the mean zero standard absorbance from each. A standard curve was made by plotting the mean absorbance for each standard on the y-axis against the concentration on the x-axis and a best fit curve was drawn through the points on the graph. If samples have been diluted, the concentration read from the standard curve must be multiplied by the dilution factor.

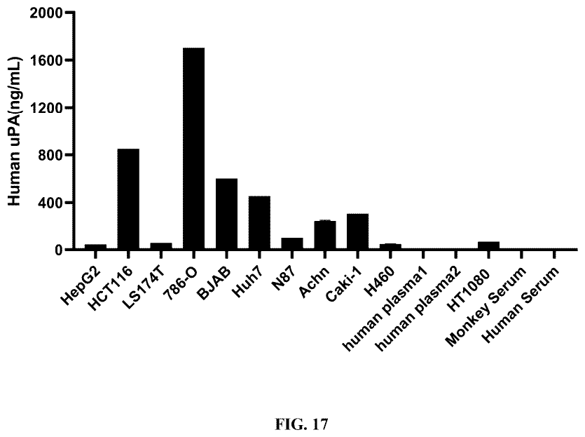

The results showed that human renal clear cell adenocarcinoma cell line 786-O, human colon cancer cell line HCT116, B cell lymphoma cell line BJAB, human hepatic carcinoma cell line Huh7, human renal cell adenocarcinoma cell line Achn, and human renal clear cell carcinoma cell line Caki-1 are tumor cells highly expressing uPA. Human gastric carcinoma cell line N87, human large cell lung cancer cell line H460, human liver cancer cell line HepG2, human colonic adenocarcinoma cell line LS174T and human fibrosarcoma cell line HT1080 are also tumor cells highly expressing uPA (FIG. 17). It is worth mentioned that there is little uPA in human plasma and serum, which greatly improves the specificity of the proBiTEs of the invention.

### Example 6. Killing of Human Cancer Cell Lines Mediated by proBiTEs

Bispecific T cell engager can simultaneously bind to specific tumor antigen and CD3+ T cell causing aggregation and activation of T cells, eventually leading to the killing of tumor cells. To evaluate the killing efficiency of proBiTE of the invention, uPA high-expressing tumor cell lines HT1080 and uPA low-expressing tumor cell lines MC38-EGFR were selected as target cells. 1.5×10⁴ target cells were seeded in 100 μL RPMI 1640 complete medium overnight. Meanwhile, frozen PBMC were revived and inoculated in 30 mL RPMI 1640 complete medium overnight. At the second day, 1.5×10⁵ PBMC in 50 μL RPMI 1640 complete medium were added. Then, 50 μL antibodies 5-fold serially diluted from 5 μg/ml were added into each well. 48 h after incubation, the medium was removed from target cells and 100 μL RPMI 1640 complete medium containing 20% CCK-8 was added and incubated 30 minutes in CO₂ incubator. Cell killing activity was measured by using microplate reader according to the manufacturer's instructions.

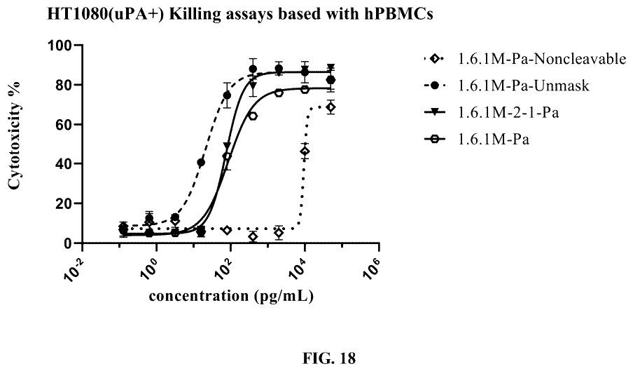

The results showed that 1.4.3-Pa, 1.6.1M-Pa and 1.6.1M-2-1-Pa all displayed potent inhibition of tumor growth (FIGS. 18-21).

FIG. 18 showed that for uPA+ cell line HT1080, 1.6.1M-Pa and 1.6.1M-2-1-Pa proBiTEs exhibited potent but slightly lower killing efficiency than 1.6.1M-Pa-Unmask BiTE, indicating that the activation of CD3 at the cellular level was slightly inhibited due to the presence of mask peptide. Compared with 1.6.1M-2-1-Pa proBiTE, 1.6.1M-Pa-Noncleavable BiTE had a weaker killing effect, indicating that the killing effect of the 1.6.1M-2-1-Pa proBiTE was caused by the exposure of CD3-binding domain of the proBiTE by cleavage of the mask peptide via uPA.

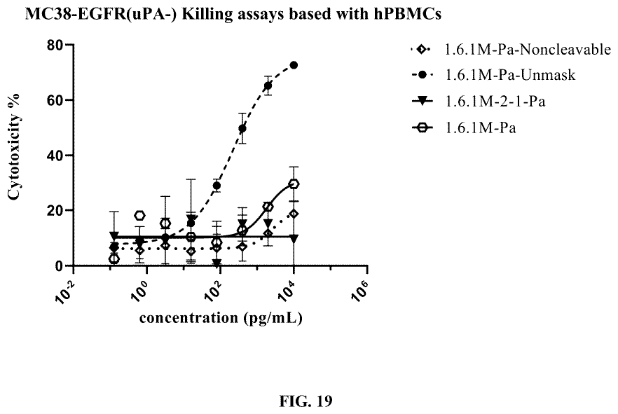

FIG. 19 showed that for uPA− cell line MC38-EGFR, 1.6.1M-Pa and 1.6.1M-2-1-Pa proBiTEs did not exhibit an effective killing effect due to the mask peptide shielding CD3-binding domain.

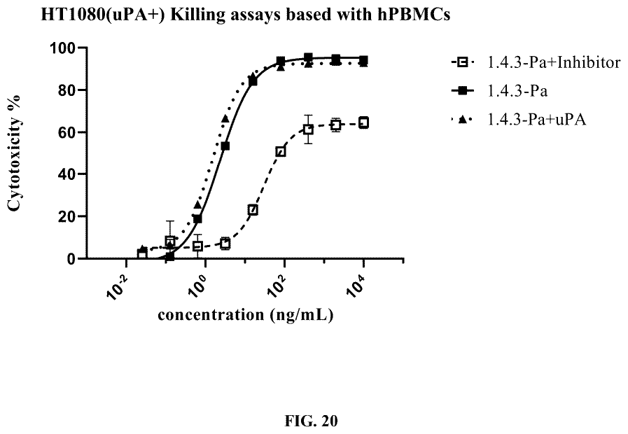

FIG. 20 showed that for uPA+ cell line HT1080, 1.4.3-Pa proBiTE exhibited potent killing efficiency no matter whether additional uPA was added or not. However, in the presence of uPA inhibitor, the killing efficiency of 1.4.3-Pa proBiTE was inhibited. This indicated that the killing efficiency of the 1.4.3-Pa proBiTE is due to the exposure of CD3-binding domain of the proBiTE by cleavage of the mask peptide via uPA.

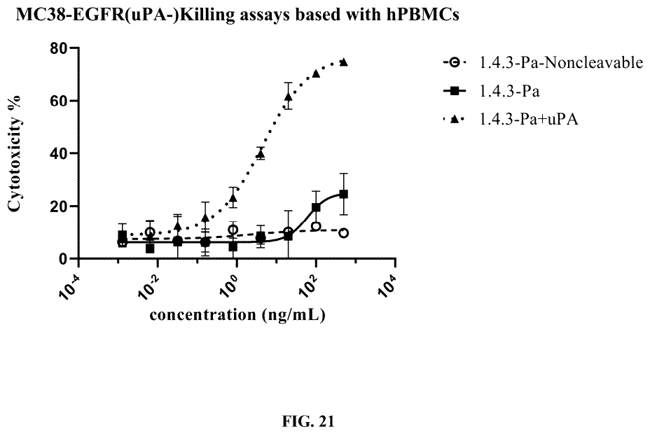

FIG. 21 showed that for uPA-cell line MC38-EGFR, 1.4.3-Pa proBiTE exhibited an effective killing effect only in the presence of uPA. This indicated that CD3-binding domain of the proBiTE can be released by cleavage of the mask peptide.

### Example 7. Inhibition of Tumor Growth in B-NDG Mice Mediated by proBiTEs

For 1.6.1M-Pa proBiTE, a total 200 μl mixture of 2×10⁶ HT-1080 cells (100 μl) and 2×10⁶ freshly isolated human PBMC (100 μl) was subcutaneously inoculated into the right abdomen of B-NDG mice. The negative control group were dosed subcutaneously with physiological saline. The positive control group was dosed with 1.6.1M-Pa-Umask BiTE (2 mg/kg). The experiment group was dosed with 1.6.1M-pa proBiTE (400 μg/kg). For 1.6.1M-2-1-Pa proBiTE, the negative control group mice were dosed subcutaneously with physiological saline. The control groups were dosed with 1.6.1M-Pa-Umask BiTE (2 mg/kg) and 1.6.1M-Pa-Noncleavable BiTE (2 mg/kg). The experiment groups were dosed with 1.6.1M-2-1-Pa proBiTE at a low dose (400 μg/kg) or a high dose (2 mg/kg). These mice were dosed twice a week. After three weeks of treatment, mice were sacrificed and tumor volume were measured. Tumor growth inhibition (TGI) rates were calculated by using the following formula: (average volume of control group-average volume of antibody treated group)/average weight of control group.

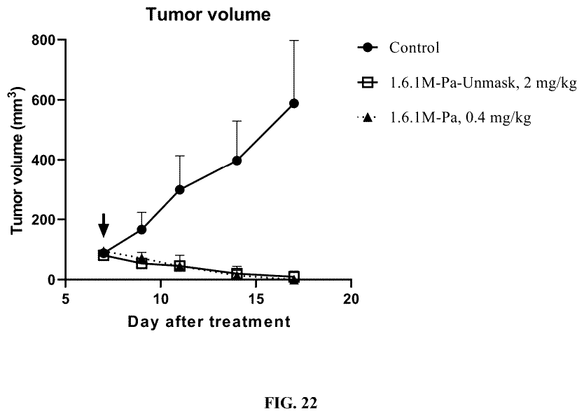

For 1.4.3M-Pa proBiTE, HT1080 tumor cells (1.8×10⁶ cells per mouse) mixed with 1.8×10⁶ human PBMCs were subcutaneously inoculated into B-NDG mice. After palpable tumors were established, randomization was performed on the basis of tumor volume and body weight. The mice were dosed intraperitoneally with 1.4.3M-Pa-Noncleavable BiTE (4 mg/kg), 1.4.3M-Pa proBiTE (0.8 mg/kg or 4 mg/kg), 1.6.1M-Pa-Unmask BiTE (0.8 mg/kg) or physiological saline as control. These mice were dosed twice or three times a week. Tumor size was measured twice per week. TGI was calculated using the following formula: TGI (%)=[1−(T−T₀)/(C−C₀)]×100, (T and T₀: tumor volumes on a specific experimental day and on the randomization day, respectively; C and C0: corresponding mean tumor volumes for the control group). Values >100% represent tumor shrinkage. The results were shown in FIGS. 22-24.

FIG. 22 showed that both 1.6.1M-Pa proBiTE and 1.6.1M-Pa-Unmask BiTE displayed potent inhibition of tumor growth, and the inhibition effect of 1.6.1M-Pa proBiTE at dose of 400 μg/kg was comparable to that of 1.6.1M-Pa-Unmask BiTE at dose of 2 mg/kg.

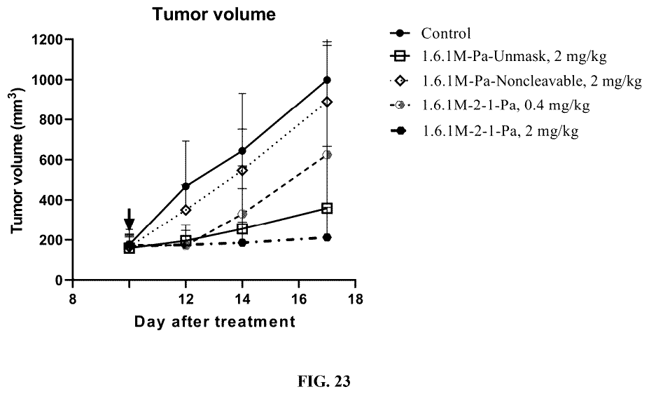

FIG. 23 showed that 1.6.1M-2-1-Pa proBiTE displayed potent inhibition of tumor growth in a dose-dependent manner.

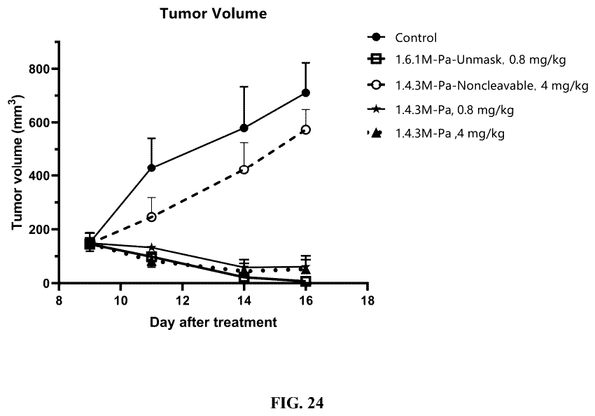

FIG. 24 showed that 1.4.3M-Pa proBiTE displayed potent inhibition of tumor growth at low and high doses.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

1. A bispecific antibody comprising
a first binding region that binds to CD3 comprising a first heavy chain variable region (VH1) and a first light chain variable region (VL1), and
a second binding region that binds to a tumor associated antigen (TAA) comprising a second heavy chain variable region (VH2) and a second light chain variable region (VL2);
wherein the VH1 comprises HCDRs 1-3 having the amino acid sequences as set forth in SEQ ID NOs: 9-11 respectively, and the VL1 comprises LCDRs 1-3 having the amino acid sequences as set forth in SEQ ID NOs: 12-14 respectively;
wherein the VH1 and/or the VL1 is linked to a mask peptide via a first linker;
wherein the mask peptide comprises the amino acid sequence of SEQ ID NO: 1 or a fragment thereof with at least 5 amino acids in length; and
wherein the first linker comprises a protease cleavage site that is specifically cleaved by a protease expressed or overexpressed in tumor microenvironment.

2. The bispecific antibody of claim 1, wherein the mask peptide comprises a fragment of at least 5 amino acids, preferably at least 7 amino acids, such as 7-15 amino acids, at N terminal of SEQ ID NO: 1.

3. The bispecific antibody of claim 1, wherein the mask peptide comprises an amino acid sequence as set forth in any of SEQ ID NOs: 1-4, or any combination thereof.

4. The bispecific antibody of claim 3, wherein the mask peptide comprises:
M1-L-M2,
wherein M1 and M2 are each independently comprises an amino acid sequence as set forth in any of SEQ ID NOs: 1-4, and L is an optional linker,
preferably the linker is (GGGS)n or (GGGGS)n, wherein n is an integer selected from 1-4.

5. The bispecific antibody of claim 4, wherein the mask peptide comprises an amino acid sequence selected from SEQ ID NOs: 5-8.

6. (canceled)

7. The bispecific antibody of claim 1, wherein the VH1 comprises an amino acid sequence as set forth in SEQ ID NO: 15 or an amino acid sequence having at least 80%, at least 85%, at least 90%, at least 95%, at least 98%, or at least 99% sequence identity to SEQ ID NO: 15, and the VL1 comprises an amino acid sequence as set forth in SEQ ID NO: 16 or an amino acid sequence having at least 80%, at least 85%, at least 90%, at least 95%, at least 98%, or at least 99% sequence identity to SEQ ID NO: 16.

8. The bispecific antibody of claim 1, wherein the protease is selected from the group consisting of uPA, MMP (e.g., MMP2, MMP9, MMP10, MMP11, MMP12, MMP13, MMP14, MMP15), matriptase B, cathepsin B, cathepsin D, collagenase and stromelysin; or wherein the protease cleavage site comprises an amino acid sequence selected from SEQ ID NOs: 17-20; RRXaa, LXaa, AXaa, FXaa or WXaa, wherein Xaa is any amino acid; or FXaa, YXaa or LXaa, wherein Xaa is any amino acid, preferably is not Ala or Val.

9. (canceled)

10. The bispecific antibody of claim 1, wherein the first linker comprises two or more protease cleavage sites connected in tandem.

11. The bispecific antibody of claim 1, wherein the first linker comprises an amino acid sequence selected from SEQ ID NOs: 21-30.

12. The bispecific antibody of claim 1, comprising
(i) a first polypeptide chain comprising, from N terminal to C terminal, the VH1, an heavy chain constant region 1 (CH1), an heavy chain constant region 2 (CH2), and an heavy chain constant region 3 (CH3); and
(ii) a second polypeptide chain comprising, from N terminal to C terminal, the VL1, a light chain constant region (CL), an heavy chain constant region 2 (CH2), and an heavy chain constant region 3 (CH3);
wherein the VH1 and/or the VL1, at its N terminal, is linked to the mask peptide via the first linker; and
(1) wherein the second binding region comprises a scFv comprising the VH2 and the VL2, wherein the scFv is linked to the C terminal of the first polypeptide chain and/or the second polypeptide chain via a linker, or wherein the scFv is linked to the N terminal of the VH1 via a linker and the mask peptide is linked to the N terminal of the VL1 via the first linker, or wherein the scFv is linked to the N terminal of the VL1 via a linker and the mask peptide is linked to the N terminal of the VH1 via the first linker; or
(2) wherein the VH2 is linked to the C terminal of the first polypeptide chain via a linker and the VL2 is linked to the C terminal of the second polypeptide chain via a linker, or wherein the VL2 is linked to the C terminal of the first polypeptide chain via a linker and the VH2 is linked to the C terminal of the second polypeptide chain via a linker; or
(3) wherein the VH2 is linked to the C terminal of the VH1 via a linker and the VL2 is linked to the C terminal of the VL1 via a linker, or wherein the VH2 is linked to the C terminal of the VL1 via a linker and the VL2 is linked to the C terminal of the VH1 via a linker.

13. The bispecific antibody of claim 12, wherein
the first polypeptide chain comprises, from N terminal to C terminal, VH1-linker-VH2-CH1-CH2-CH3; and the second polypeptide chain comprises, from N terminal to C terminal, VL1-linker-VL2-CL-CH2-CH3; or
the first polypeptide chain comprises, from N terminal to C terminal, VH1-CH1-CH2-CH3-linker-VH2; and the second polypeptide chain comprises, from N terminal to C terminal, VL1-CL-CH2-CH3-linker-VL2; or
the first polypeptide chain comprises, from N terminal to C terminal, VH1-CH1-CH2-CH3; and the second polypeptide chain comprises, from N terminal to C terminal, scFv-linker-VL1-CL-CH2-CH3; or
the first polypeptide chain comprises, from N terminal to C terminal, VH1-CH1-CH2-CH3-linker-scFv; and the second polypeptide chain comprises, from N terminal to C terminal, VL1-CL-CH2-CH3-linker-scFv.

14. The bispecific antibody of claim 13, wherein the first polypeptide chain comprises, from N terminal to C terminal, the mask peptide-the first linker-VH1-linker-VH2-CH1-CH2-CH3; and the second polypeptide chain comprises, from N terminal to C terminal, VL1-linker-VL2-CL-CH2-CH3; or
the first polypeptide chain comprises, from N terminal to C terminal, the mask peptide-the first linker-VH1-CH1-CH2-CH3; and the second polypeptide chain comprises, from N terminal to C terminal, scFv-linker-VL1-CL-CH2-CH3.

15. (canceled)

16. (canceled)

17. The bispecific antibody of claim 1, wherein the TAA is selected from the group consisting of EGFR, EpCAM, HER2, PSMA, gpA33, CD276, CEA, CD19, CD20, CD22, CD30, CD33, CD123, FLT3 and BCMA.

18. The bispecific antibody of claim 17, wherein the TAA is EGFR, and wherein
the VH2 comprises HCDRs 1-3 having the amino acid sequences as set forth in SEQ ID NOs: 31-33 respectively, and the VL2 comprises LCDRs 1-3 having the amino acid sequences as set forth in SEQ ID NOs: 34-36 respectively; or
the VH2 comprises HCDRs 1-3 having the amino acid sequences as set forth in SEQ ID NOs: 37-39 respectively, and the VL2 comprises LCDRs 1-3 having the amino acid sequences as set forth in SEQ ID NOs: 40-42 respectively.

19. The bispecific antibody of claim 18, wherein the VH2 comprises an amino acid sequence as set forth in SEQ ID NO: 43 or an amino acid sequence having at least 80%, at least 85%, at least 90%, at least 95%, at least 98%, or at least 99% sequence identity to SEQ ID NO: 43, and the VL2 comprises an amino acid sequence as set forth in SEQ ID NO: 44 or an amino acid sequence having at least 80%, at least 85%, at least 90%, at least 95%, at least 98%, or at least 99% sequence identity to SEQ ID NO: 44; or
the VH2 comprises an amino acid sequence as set forth in SEQ ID NO: 45 or an amino acid sequence having at least 80%, at least 85%, at least 90%, at least 95%, at least 98%, or at least 99% sequence identity to SEQ ID NO: 45, and the VL2 comprises an amino acid sequence as set forth in SEQ ID NO: 46 or an amino acid sequence having at least 80%, at least 85%, at least 90%, at least 95%, at least 98%, or at least 99% sequence identity to SEQ ID NO: 46.

20. The bispecific antibody of claim 19, wherein the second binding region comprises a scFv comprising an amino acid sequence as set forth in SEQ ID NO: 75 or SEQ ID NO: 76 or an amino acid sequence having at least 80%, at least 85%, at least 90%, at least 95%, at least 98%, or at least 99% sequence identity to SEQ ID NO: 75 or SEQ ID NO: 76.

21. The bispecific antibody of claim 13, wherein one or both of the CH3 comprise at least one amino acid mutation that is capable of decreasing homodimerization between the first and second polypeptide chains; and/or
wherein one or both of the CH2 comprise at least one amino acid mutation that is capable of decreasing the effector function of the bispecific antibody, preferably the at least one mutation is selected from L234A, L235A, G237A, or any combination thereof.

22. (canceled)

23. The bispecific antibody of claim 1, wherein the bispecific antibody comprises:
a first polypeptide chain comprising an amino acid as set forth in SEQ ID NO: 51 or an amino acid sequence having at least 80%, at least 85%, at least 90%, at least 95%, at least 98%, or at least 99% sequence identity to SEQ ID NO: 51, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 52 or an amino acid sequence having at least 80%, at least 85%, at least 90%, at least 95%, at least 98%, or at least 99% sequence identity to SEQ ID NO: 52, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 53 or an amino acid sequence having at least 80%, at least 85%, at least 90%, at least 95%, at least 98%, or at least 99% sequence identity to SEQ ID NO: 53, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 54 or an amino acid sequence having at least 80%, at least 85%, at least 90%, at least 95%, at least 98%, or at least 99% sequence identity to SEQ ID NO: 54, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 55 or an amino acid sequence having at least 80%, at least 85%, at least 90%, at least 95%, at least 98%, or at least 99% sequence identity to SEQ ID NO: 55, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 56 or an amino acid sequence having at least 80%, at least 85%, at least 90%, at least 95%, at least 98%, or at least 99% sequence identity to SEQ ID NO: 56, and a second polypeptide chain comprising an amino acid as set forth in SEQ ID NO: 50 or an amino acid sequence having at least 80%, at least 85%, at least 90%, at least 95%, at least 98%, or at least 99% sequence identity to SEQ ID NO: 50; or
a first polypeptide chain comprising an amino acid as set forth in SEQ ID NO: 52 or an amino acid sequence having at least 80%, at least 85%, at least 90%, at least 95%, at least 98%, or at least 99% sequence identity to SEQ ID NO: 52, and a second polypeptide chain comprising an amino acid as set forth in SEQ ID NO: 57 or an amino acid sequence having at least 80%, at least 85%, at least 90%, at least 95%, at least 98%, or at least 99% sequence identity to SEQ ID NO: 57; or
a first polypeptide chain comprising an amino acid as set forth in SEQ ID NO: 58 or an amino acid sequence having at least 80%, at least 85%, at least 90%, at least 95%, at least 98%, or at least 99% sequence identity to SEQ ID NO: 58, and a second polypeptide chain comprising an amino acid as set forth in SEQ ID NO: 59 or an amino acid sequence having at least 80%, at least 85%, at least 90%, at least 95%, at least 98%, or at least 99% sequence identity to SEQ ID NO: 59; or
a first polypeptide chain comprising an amino acid as set forth in SEQ ID NO: 60 or an amino acid sequence having at least 80%, at least 85%, at least 90%, at least 95%, at least 98%, or at least 99% sequence identity to SEQ ID NO: 60, and a second polypeptide chain comprising an amino acid as set forth in SEQ ID NO: 61 or an amino acid sequence having at least 80%, at least 85%, at least 90%, at least 95%, at least 98%, or at least 99% sequence identity to SEQ ID NO: 61; or
a first polypeptide chain comprising an amino acid as set forth in SEQ ID NO: 62 or an amino acid sequence having at least 80%, at least 85%, at least 90%, at least 95%, at least 98%, or at least 99% sequence identity to SEQ ID NO: 62, and a second polypeptide chain comprising an amino acid as set forth in SEQ ID NO: 63 or an amino acid sequence having at least 80%, at least 85%, at least 90%, at least 95%, at least 98%, or at least 99% sequence identity to SEQ ID NO: 63; or
a first polypeptide chain comprising an amino acid as set forth in SEQ ID NO: 64 or an amino acid sequence having at least 80%, at least 85%, at least 90%, at least 95%, at least 98%, or at least 99% sequence identity to SEQ ID NO: 64, and a second polypeptide chain comprising an amino acid as set forth in SEQ ID NO: 65 or an amino acid sequence having at least 80%, at least 85%, at least 90%, at least 95%, at least 98%, or at least 99% sequence identity to SEQ ID NO: 65; or
a first polypeptide chain comprising an amino acid as set forth in SEQ ID NO: 66 or an amino acid sequence having at least 80%, at least 85%, at least 90%, at least 95%, at least 98%, or at least 99% sequence identity to SEQ ID NO: 66, and a second polypeptide chain comprising an amino acid as set forth in SEQ ID NO: 67 or an amino acid sequence having at least 80%, at least 85%, at least 90%, at least 95%, at least 98%, or at least 99% sequence identity to SEQ ID NO: 67; or
a first polypeptide chain comprising an amino acid as set forth in SEQ ID NO: 66 or an amino acid sequence having at least 80%, at least 85%, at least 90%, at least 95%, at least 98%, or at least 99% sequence identity to SEQ ID NO: 66, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 69 or an amino acid sequence having at least 80%, at least 85%, at least 90%, at least 95%, at least 98%, or at least 99% sequence identity to SEQ ID NO: 69, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 70 or an amino acid sequence having at least 80%, at least 85%, at least 90%, at least 95%, at least 98%, or at least 99% sequence identity to SEQ ID NO: 70, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 71 or an amino acid sequence having at least 80%, at least 85%, at least 90%, at least 95%, at least 98%, or at least 99% sequence identity to SEQ ID NO: 71, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 72 or an amino acid sequence having at least 80%, at least 85%, at least 90%, at least 95%, at least 98%, or at least 99% sequence identity to SEQ ID NO: 72, and a second polypeptide chain comprising an amino acid as set forth in SEQ ID NO: 68 or an amino acid sequence having at least 80%, at least 85%, at least 90%, at least 95%, at least 98%, or at least 99% sequence identity to SEQ ID NO: 68; or
a first polypeptide chain comprising an amino acid as set forth in SEQ ID NO: 73 or an amino acid sequence having at least 80%, at least 85%, at least 90%, at least 95%, at least 98%, or at least 99% sequence identity to SEQ ID NO: 73, and a second polypeptide chain comprising an amino acid as set forth in SEQ ID NO: 74 or an amino acid sequence having at least 80%, at least 85%, at least 90%, at least 95%, at least 98%, or at least 99% sequence identity to SEQ ID NO: 74; or
a first polypeptide chain comprising an amino acid as set forth in SEQ ID NO: 77 or an amino acid sequence having at least 80%, at least 85%, at least 90%, at least 95%, at least 98%, or at least 99% sequence identity to SEQ ID NO: 77, and a second polypeptide chain comprising an amino acid as set forth in SEQ ID NO: 78 or an amino acid sequence having at least 80%, at least 85%, at least 90%, at least 95%, at least 98%, or at least 99% sequence identity to SEQ ID NO: 78.

24. A nucleic acid comprising a nucleotide sequence encoding the bispecific antibody according to claim 1.

25. (canceled)

26. (canceled)

27. A pharmaceutical composition comprising (i) the bispecific antibody according to claim 1; and (ii) a pharmaceutically acceptable carrier or excipient.

28. (canceled)

29. (canceled)

30. (canceled)

31. (canceled)

32. (canceled)

33. A method of treating a cancer in a subject, comprising administering to the subject an effective amount of the bispecific antibody according to claim 1.

34. The method according to claim 33, wherein the TAA is EGFR and the cancer is an EGFR positive cancer, or the cancer is selected from the group consisting of fibrosarcoma, colon cancer, colorectal cancer, liver cancer, kidney cancer, lung cancer, gastric cancer, ovarian cancer, breast cancer, pancreatic cancer, head and neck cancer, glioblastoma, and prostatic cancer.

35. (canceled)

36. (canceled)

37. (canceled)

38. (canceled)


SEQUENCE LISTING (96 sequences)

SEQ ID NO: 1 - protein, 22 aa, synthetic construct
  REGION 1..22: note = Mask4
QDGNEEMGGITQTPYKVSISGT

SEQ ID NO: 2 - protein, 15 aa, synthetic construct
  REGION 1..15: note = Mask3
QDGNEEMGGITQTPY

SEQ ID NO: 3 - protein, 10 aa, synthetic construct
  REGION 1..10: note = Mask2
QDGNEEMGGI

SEQ ID NO: 4 - protein, 7 aa, synthetic construct
  REGION 1..7: note = Mask1
QDGNEEM

SEQ ID NO: 5 - protein, 18 aa, synthetic construct
  REGION 1..18: note = Mask1‘
QDGNEEMGGGSQDGNEEM

SEQ ID NO: 6 - protein, 21 aa, synthetic construct
  REGION 1..21: note = Mask2‘
QDGNEEMGGIGGGSQDGNEEM

SEQ ID NO: 7 - protein, 26 aa, synthetic construct
  REGION 1..26: note = Mask1'
QDGNEEMGGGSQDGNEEMGGITQTPY

SEQ ID NO: 8 - protein, 21 aa, synthetic construct
  REGION 1..21: note = Mask1’
QDGNEEMGGGSQDGNEEMGGI

SEQ ID NO: 9 - protein, 8 aa, synthetic construct
  REGION 1..8: note = Anti-CD3 HCDR1
GFTFNTYA

SEQ ID NO: 10 - protein, 10 aa, synthetic construct
  REGION 1..10: note = Anti-CD3 HCDR2
IRSKYNNYAT

SEQ ID NO: 11 - protein, 16 aa, synthetic construct
  REGION 1..16: note = Anti-CD3 HCDR3
ARHGNFGSSYVSYFAY

SEQ ID NO: 12 - protein, 9 aa, synthetic construct
  REGION 1..9: note = Anti-CD3 LCDR1
TGAVTTSNY

SEQ ID NO: 13 - skipped

SEQ ID NO: 14 - protein, 9 aa, synthetic construct
  REGION 1..9: note = Anti-CD3 LCDR3
ALWYSNLWV

SEQ ID NO: 15 - protein, 125 aa, synthetic construct
  REGION 1..125: note = Anti-CD3 VH
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSS

SEQ ID NO: 16 - protein, 110 aa, synthetic construct
  REGION 1..110: note = Anti-CD3 VL
EIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIK

SEQ ID NO: 17 - protein, 8 aa, synthetic construct
  REGION 1..8: note = uPA protease cleavage site
LSGRSDNH

SEQ ID NO: 18 - protein, 6 aa, synthetic construct
  REGION 1..6: note = MMP2 protease cleavage site
PLGLAG

SEQ ID NO: 19 - protein, 5 aa, synthetic construct
  REGION 1..5: note = MatB protease cleavage site
PMAKK

SEQ ID NO: 20 - protein, 10 aa, synthetic construct
  REGION 1..10: note = MMP9 protease cleavage site
PGPAGMKGLP

SEQ ID NO: 21 - protein, 24 aa, synthetic construct
  REGION 1..24: note = first linker
GGGSGGGSGGGSGLSGRSDNHGGS

SEQ ID NO: 22 - protein, 26 aa, synthetic construct
  REGION 1..26: note = first linker
GGGGGSGGGSGGGSGLSGRSDNHGGS

SEQ ID NO: 23 - protein, 22 aa, synthetic construct
  REGION 1..22: note = first linker
GGGGGSGGGSGLSGRSDNHGGS

SEQ ID NO: 24 - protein, 29 aa, synthetic construct
  REGION 1..29: note = first linker
GGGGSGGGSGGGSGLSGRSDNHGGSSGGS

SEQ ID NO: 25 - protein, 23 aa, synthetic construct
  REGION 1..23: note = first linker
GGGSGGGGSGLSGRSDNHGGGGS

SEQ ID NO: 26 - protein, 27 aa, synthetic construct
  REGION 1..27: note = first linker
GGGGSGGGSGGGSGLSGRSDNHGGGGS

SEQ ID NO: 27 - protein, 31 aa, synthetic construct
  REGION 1..31: note = first linker
GGGSGGGGSGLSGRSDNHGGLSGRSDNHGGS

SEQ ID NO: 28 - protein, 29 aa, synthetic construct
  REGION 1..29: note = first linker
GGGSGGGGSGLSGRSDNHGGPLGLAGGGS

SEQ ID NO: 29 - protein, 28 aa, synthetic construct
  REGION 1..28: note = first linker
GGGSGGGGSGLSGRSDNHGGPMAKKGGS

SEQ ID NO: 30 - protein, 33 aa, synthetic construct
  REGION 1..33: note = first linker
GGGSGGGGSGLSGRSDNHGGPGPAGMKGLPGGS

SEQ ID NO: 31 - protein, 8 aa, synthetic construct
  REGION 1..8: note = anti-EGFR HCDR1
GFTFSSYA

SEQ ID NO: 32 - protein, 8 aa, synthetic construct
  REGION 1..8: note = anti-EGFR HCDR2
ISYDGSNK

SEQ ID NO: 33 - protein, 12 aa, synthetic construct
  REGION 1..12: note = anti-EGFR HCDR3
ARGYVARGDFDY

SEQ ID NO: 34 - protein, 6 aa, synthetic construct
  REGION 1..6: note = anti-EGFR LCDR1
QSMSRW

SEQ ID NO: 35 - skipped

SEQ ID NO: 36 - protein, 11 aa, synthetic construct
  REGION 1..11: note = anti-EGFR LCDR3
QQLDSFPFSFT

SEQ ID NO: 37 - protein, 10 aa, synthetic construct
  REGION 1..10: note = Panitumumab HCDR1
GGSVSSGDYY

SEQ ID NO: 38 - protein, 7 aa, synthetic construct
  REGION 1..7: note = Panitumumab HCDR2
IYYSGNT

SEQ ID NO: 39 - protein, 11 aa, synthetic construct
  REGION 1..11: note = Panitumumab HCDR3
VRDRVTGAFDI

SEQ ID NO: 40 - protein, 6 aa, synthetic construct
  REGION 1..6: note = Panitumumab LCDR1
QDISNY

SEQ ID NO: 41 - skipped

SEQ ID NO: 42 - protein, 9 aa, synthetic construct
  REGION 1..9: note = Panitumumab LCDR3
QHFDHLPLA

SEQ ID NO: 43 - protein, 119 aa, synthetic construct
  REGION 1..119: note = Anti-EGFR VH
EVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSS

SEQ ID NO: 44 - protein, 109 aa, synthetic construct
  REGION 1..109: note = Anti-EGFR VL
DVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLK

SEQ ID NO: 45 - protein, 119 aa, synthetic construct [sequence omitted]

SEQ ID NO: 46 - protein, 107 aa, synthetic construct [sequence omitted]

SEQ ID NO: 47 - protein, 15 aa, synthetic construct
  REGION 1..15: note = Linker
GGGSSGGGGSGGGGS

SEQ ID NO: 48 - protein, 15 aa, synthetic construct
  REGION 1..15: note = Linker
GGGGSGGGGSGGGGS

SEQ ID NO: 49 - protein, 20 aa, synthetic construct
  REGION 1..20: note = Linker
GGGGSGGGGSGGGGSGGGGS

SEQ ID NO: 50 - protein, 562 aa, synthetic construct
  REGION 1..562: note = 1.1-1.4.3 light chain
EIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKGGGGSGGGGSGGGGSDVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 51 - protein, 617 aa, synthetic construct
  REGION 1..617: note = 1.1 heavy chain
QDGNEEMGGIGGGSGGGSGGGSGLSGRSDNHGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 52 - protein, 622 aa, synthetic construct
  REGION 1..622: note = 1.2 heavy chain
QDGNEEMGGITQTPYGGGSGGGSGGGSGLSGRSDNHGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 53 - protein, 629 aa, synthetic construct
  REGION 1..629: note = 1.3 heavy chain
QDGNEEMGGITQTPYKVSISGTGGGSGGGSGGGSGLSGRSDNHGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 54 - protein, 630 aa, synthetic construct
  REGION 1..630: note = 1.4 heavy chain
QDGNEEMGGIGGGSQDGNEEMGGGGGSGGGSGGGSGLSGRSDNHGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 55 - protein, 633 aa, synthetic construct
  REGION 1..633: note = 1.4.1 heavy chain
QDGNEEMGGGSQDGNEEMGGITQTPYGGGSGGGSGGGSGLSGRSDNHGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 56 - protein, 626 aa, synthetic construct
  REGION 1..626: note = 1.4.3 heavy chain
QDGNEEMGGIGGGSQDGNEEMGGGGGSGGGSGLSGRSDNHGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 57 - protein, 601 aa, synthetic construct
  REGION 1..601: note = 1.5 light chain
QDGNEEMGGITQTPYGGGSGGGSGGGSGLSGRSDNHGGSEIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKGGGGSGGGGSGGGGSDVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 58 - protein, 630 aa, synthetic construct
  REGION 1..630: note = 1.6 heavy chain
QDGNEEMGGGSQDGNEEMGGGGSGGGSGGGSGLSGRSDNHGGSSGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGKGGGGSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSS

SEQ ID NO: 59 - protein, 562 aa, synthetic construct
  REGION 1..562: note = 1.6 light chain
EIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGKGGGGSGGGGSGGGGSDVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLK

SEQ ID NO: 60 - protein, 493 aa, synthetic construct
  REGION 1..493: note = 1.6.1 heavy chain
QDGNEEMGGGSQDGNEEMGGIGGGSGGGGSGLSGRSDNHGGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 61 - protein, 706 aa, synthetic construct
  REGION 1..706: note = 1.6.1 light chain
EVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSGGGGSGGGGSGGGGSGGGGSDVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLKGGGGSGGGGSGGGGSGGGGSEIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 62 - protein, 754 aa, synthetic construct
  REGION 1..754: note = 1.7 heavy chain
QDGNEEMGGGSQDGNEEMGGGGSGGGSGGGSGLSGRSDNHGGSSGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGKGGGGSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSGGGGSGGGGSGGGGSDVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLK

SEQ ID NO: 63 - protein, 696 aa, synthetic construct
  REGION 1..696: note = 1.7 light chain
EIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGKGGGGSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSGGGGSGGGGSGGGGSDVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLK

SEQ ID NO: 64 - protein, 762 aa, synthetic construct
  REGION 1..762: note = 1.7.1 heavy chain
QDGNEEMGGGSQDGNEEMGGGGSGGGSGGGSGLSGRSDNHGGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGKGGGGSGGGGSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSGGGGSGGGGSGGGGSGGGGSDVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLK

SEQ ID NO: 65 - protein, 706 aa, synthetic construct
  REGION 1..706: note = 1.7.1 light chain
EIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGKGGGGSGGGGSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSGGGGSGGGGSGGGGSGGGGSDVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLK

SEQ ID NO: 66 - protein, 493 aa, synthetic construct
  REGION 1..493: note = 1.6.1M/1.6.1M-Pa heavy chain
QDGNEEMGGGSQDGNEEMGGIGGGSGGGGSGLSGRSDNHGGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 67 - protein, 706 aa, synthetic construct
  REGION 1..706: note = 1.6.1M light chain
EVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSGGGGSGGGGSGGGGSGGGGSDVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLKGGGGSGGGGSGGGGSGGGGSEIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 68 - protein, 704 aa, synthetic construct
  REGION 1..704: note = 1.6.1M-Pa/1.6.1M-2-1-Pa/1.6.1M-2-2-Pa/1.6.1M-2-3-Pa /1.6.1M-2-4-Pa light chain
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSGGGGSEIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 69 - protein, 501 aa, synthetic construct
  REGION 1..501: note = 1.6.1M-2-1-Pa heavy chain
QDGNEEMGGGSQDGNEEMGGIGGGSGGGGSGLSGRSDNHGGLSGRSDNHGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 70 - protein, 499 aa, synthetic construct
  REGION 1..499: note = 1.6.1M-2-2-Pa heavy chain
QDGNEEMGGGSQDGNEEMGGIGGGSGGGGSGLSGRSDNHGGPLGLAGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 71 - protein, 498 aa, synthetic construct
  REGION 1..498: note = 1.6.1M-2-3-Pa heavy chain
QDGNEEMGGGSQDGNEEMGGIGGGSGGGGSGLSGRSDNHGGPMAKKGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 72 - protein, 503 aa, synthetic construct
  REGION 1..503: note = 1.6.1M-2-4-Pa heavy chain
QDGNEEMGGGSQDGNEEMGGIGGGSGGGGSGLSGRSDNHGGPGPAGMKGLPGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 73 - protein, 626 aa, synthetic construct
  REGION 1..626: note = 1.4.3M-Pa heavy chain
QDGNEEMGGIGGGSQDGNEEMGGGGGSGGGSGLSGRSDNHGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 74 - protein, 560 aa, synthetic construct
  REGION 1..560: note = 1.4.3M-Pa light chain
EIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 75 - protein, 248 aa, synthetic construct
  REGION 1..248: note = anti-EGFR SMET5.2 scFv
EVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSGGGGSGGGGSGGGGSGGGGSDVVMTQSPSTLSASVGDRVTITCRASQSMSRWLAWYQQKPGKGPKLLIYATSTLQSGVPSRFSGSGSGTEFTLTISSLQPEDFATYYCQQLDSFPFSFTFGPGTKVDLK

SEQ ID NO: 76 - protein, 246 aa, synthetic construct
  REGION 1..246: note = anti-EGFR panitumumab scFv
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIK

SEQ ID NO: 77 - protein, 626 aa, synthetic construct
  REGION 1..626: note = 1.4.3Pa heavy chain
QDGNEEMGGIGGGSQDGNEEMGGGGGSGGGSGLSGRSDNHGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 78 - protein, 560 aa, synthetic construct
  REGION 1..560: note = 1.4.3Pa light chain
EIVVTQSPATLSVSPGERATLSCRSSTGAVTTSNYANWVQQKPGQAPRGLIGGANKRAPGVPARFSGSLSGDEATLTISSLQSEDFAVYYCALWYSNLWVFGQGTKLEIKGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 79 - protein, 583 aa, synthetic construct
  REGION 1..583: note = 1.1-Unmask Heavy chain
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 80 - protein, 630 aa, synthetic construct
  REGION 1..630: note = 1.4-Noncleavable Heavy chain
QDGNEEMGGIGGGSQDGNEEMGGGGGSGGGSGGGSGLSGGAGNHGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARGYVARGDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 81 - protein, 449 aa, synthetic construct
  REGION 1..449: note = 1.6.1M-Unmask Heavy chain
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 82 - protein, 492 aa, synthetic construct
  REGION 1..492: note = 1.6.1M-Noncleavable Heavy chain
QDGNEEMGGGSQDGNEEMGGIGGGSGGGGSGLSGAGNHGGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 83 - protein, 449 aa, synthetic construct; identical to SEQ ID NO: 81
  REGION 1..449: note = 1.6.1M-Pa-Unmask Heavy chain

SEQ ID NO: 84 - protein, 492 aa, synthetic construct; identical to SEQ ID NO: 82
  REGION 1..492: note = 1.6.1M-Pa-Noncleavable Heavy chain

SEQ ID NO: 85 - protein, 618 aa, synthetic construct
  REGION 1..618: note = 1.4.3-Pa-Noncleavable Heavy chain
QDGNEEMGGIGGGSQDGNEEMGGGGGSGGGSGGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 86 - protein, 583 aa, synthetic construct
  REGION 1..583: note = 1.4.3M-Pa-Unmask Heavy chain
EVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 87 - protein, 617 aa, synthetic construct
  REGION 1..617: note = 1.4.3M-Pa-Noncleavable Heavy chain
QDGNEEMGGIGGGSQDGNEEMGGGGGSGGGSGGSEVQLVESGGGLVQPGGSLRLSCAASGFTFNTYAMNWVRQAPGKGLEWVARIRSKYNNYATYYADSVKGRFTISRDDSKNTLYLQMNSLRAEDTAVYYCARHGNFGSSYVSYFAYWGQGTTVTVSSGGGSSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLLCLVKGFYPSDIAVEWESNGQPENNYKTTPPVLDSDGSFFLHSKLTVDKSRWQQGNVFSCSVMHEALHNHYTQKSLSLSPGK

SEQ ID NO: 88 - protein, 4 aa, synthetic construct
  REGION 1..4: note = linker
GGGS

SEQ ID NO: 89 - protein, 8 aa, synthetic construct
  REGION 1..8: note = linker
GGGSGGGS

SEQ ID NO: 90 - protein, 12 aa, synthetic construct
  REGION 1..12: note = linker
GGGSGGGSGGGS

SEQ ID NO: 91 - protein, 16 aa, synthetic construct
  REGION 1..16: note = linker
GGGSGGGSGGGSGGGS

SEQ ID NO: 92 - protein, 5 aa, synthetic construct
  REGION 1..5: note = linker
GGGGS

SEQ ID NO: 93 - protein, 10 aa, synthetic construct
  REGION 1..10: note = linker
GGGGSGGGGS

SEQ ID NO: 94 - protein, 10 aa, synthetic construct
  REGION 1..10: note = linker
GGGSSGGGGS

SEQ ID NO: 95 - protein, 20 aa, synthetic construct
  REGION 1..20: note = linker
GGGSSGGGGSGGGGSGGGGS

SEQ ID NO: 96 - protein, 25 aa, synthetic construct
  REGION 1..25: note = linker
GGGSSGGGGSGGGGSGGGGSGGGGS